(19) United States
(12) Patent Application Publication
(10) Pub. No.: US 2026/0158166 A1
(43) Pub. Date: Jun. 11, 2026

(54) PRODUCTION OF RECOMBINANT AAV

(71) Applicant: Genzyme Corporation, Cambridge, MA (US)

(72) Inventors: Meghana Kodalali BASAVARAJ, Chelmsford, MA (US); Jarrod DEAN, Medway, MA (US); Prasad KESANAKURTI, Lexington, MA (US); Christian MUELLER, Concord, MA (US); Catherine O’RIORDAN, Waban, MA (US)

(73) Assignee: Genzyme Corporation, Cambridge, MA (US)

(21) Appl. No.: 19/332,406

(22) Filed: Sep. 18, 2025

Related U.S. Application Data
(60) Provisional application No. 63/696,324, filed on Sep. 18, 2024.

(51) Int. Cl. A61K 48/00; C07K 16/18; C12N 7/00

(52) U.S. Cl. CPC A61K 48/005; A61K 48/0075; C07K 16/18; C12N 7/00; C07K 2317/565; C07K 2317/622; C12N 2750/14143; C12N 2750/14152; C12N 2830/15

(57) ABSTRACT

The present disclosure provides methods of producing in a single cell a mixed population of recombinant adeno-associated viruses for expressing two or more transgenes and methods of producing recombinant adeno-associated viruses containing self-complementary genomes. Also provided are methods of treating diseases using the rAAV compositions produced herein.

### CROSS REFERENCE TO RELATED APPLICATION

The present application claims priority from 63/696,324, filed Sep. 18, 2024, the contents of which are incorporated herein by reference in their entirety.

### SEQUENCE LISTING

The application contains a Sequence Listing which has been submitted electronically in .XML format and is hereby incorporated by reference in its entirety. Said .XML copy, created on Sep. 17, 2025, is named “122548.WO017.xml” and is 65,140 bytes in size.

### BACKGROUND OF THE INVENTION

Adeno-associated virus (AAV) is a type of DNA virus from the Parvoviridae family and has a small, non-enveloped, icosahedral capsid that measures 26 nm in diameter. The virus contains a 4.7-kilobase linear single-stranded DNA genome with two main genes (Rep and Cap) and a 145-nucleotide inverted terminal repeat (ITR) at each end (Handa and Carter, J Biol Chem. (1979) 254:6603-10 and Dela Maza and Carter, J Biol Chem. (1980) 255:319403203). The Rep gene encodes replicases and the Cap gene encodes capsid proteins. By swapping out the Rep and Cap genes for a desired transgene, AAV can be converted into a recombinant AAV (rAAV) for gene delivery.

Recombinant AAV-based viral vectors can serve as efficient vehicles for in vivo human gene therapy. Recombinant AAV may be produced in human cell lines (e.g., HEK293 cells) or insect cells (e.g., in the Sf9/baculovirus system). Human cells are the natural host of AAV, but AAV does not replicate without the help of a helper virus (e.g., an adenovirus or a herpes simplex virus) that infects the same cell. Recombinant AAV may be produced in a stable producer cell line (PCL) or by transient transfection of host cells. For example, a human PCL can be established by stably integrating in its genome AAV Rep and Cap genes as well as a template for an rAAV genome; replication and packaging of rAAV may be initiated by infecting the cells with a helper virus. In a transient transfection (TTx) approach, human host cells may be transiently transfected with plasmids carrying the AAV Rep and Cap genes, the template for an rAAV genome, and coding sequences for helper components (e.g., adenoviral E4, E2 and VA proteins).

High costs of rAAV production add to the challenges faced by gene therapy. Furthermore, rAAV has a size limitation—it cannot carry a transgene that is longer than its natural genome size of about 4.7 kb. Thus, there remains a need to overcome these limitations in using rAAV for gene therapy.

### SUMMARY OF THE INVENTION

The present disclosure provides a method of producing a recombinant adeno-associated virus (AAV) composition comprising a first AAV having a self-complementary recombinant genome comprising a first transgene and a second AAV having a self-complementary recombinant genome comprising a second transgene, the method comprising: introducing into a host cell an exogenous DNA encoding a template AAV genome comprising an expression cassette flanked by AAV inverted terminal repeats (ITRs), wherein the expression cassette comprises the first and second transgenes placed in opposite directions and separated by a bidirectional promoter, and wherein the bidirectional promoter comprises palindromic sequences, and culturing the host cell under conditions for AAV replication, wherein a recombinant AAV composition is produced. In some embodiments, the method further comprises isolating the recombinant AAV composition produced from the host cell.

In some embodiments, the host cell is a mammalian cell engineered to express an AAV Rep gene and an AAV Cap gene. In further embodiments, the exogenous DNA, the AAV Rep gene and the AAV Cap gene are stably integrated into the host cell genome and the AAV replication is initiated by infecting the host cell with an adenovirus or a herpes simplex 1 virus. In other embodiments, the exogenous DNA, the AAV Rep gene, the AAV Cap gene, and adenoviral helper genes are introduced into the host cell by transient transfection, wherein the adenoviral helper genes comprise E4, E2a, and VA genes.

In some embodiments, the ratio of the first AAV to the second AAV in the composition is about 0.5:1 to about 2:1, optionally about 1:1.

In some embodiments, the bidirectional promoter is a minCBA promoter comprising a pair of chicken β-actin (CBA) promoters placed in opposite direction and separated by a CMV enhancer. In some embodiments, the CMV enhancer comprises SEQ ID NO:8, or a nucleic acid sequence at least 85% identical thereto. In further embodiments, the bidirectional promoter comprises SEQ ID NO:1 or 30, or a nucleic acid sequence at least 85% identical thereto. In some embodiments, the AAV ITRs are AAV2 ITRs.

In some embodiments, the first transgene comprises a portion a full-length gene and the second transgene comprises the remainder of the full-length gene, wherein the full-length gene is 4.5 to 9 kb long, and said portion and said remainder each are no longer than 4.8 kb, (a) wherein the first transgene comprises a splice donor at the 3′ end of its coding region and the second transgene comprises a splice acceptor at the 5′ end of its coding region, and wherein the splice donor and the splice acceptor promote generation of an RNA transcript of the full-length gene upon co-delivery of the first and second AAVs into a target cell; or (b) wherein the 3′ coding region of the first transgene and the 5′ coding region of the second transgene overlap by 10 or more nucleotides, and wherein the overlap region promotes generation of an RNA transcript of the full-length gene upon co-delivery of the first and second AAVs into a target cell.

In some embodiments, the first and second transgenes each code for a different therapeutic protein. In further embodiments, the first transgene encodes an anti-C1s antibody fragment, optionally an scFv or an scFab, and the second transgene encodes an anti-Bb antibody fragment, optionally an scFv or an scFab. In other embodiments, the first and second transgenes each code for the same therapeutic protein (e.g., the transgenes are identical). In further embodiments, the first and second transgenes both encode an anti-C1s antibody fragment, optionally an scFv or an scFab, or the first and second transgenes both encode an anti-Bb antibody fragment, optionally an scFv or an scFab. In certain embodiments, the anti-C1s antibody comprises HCDR1-3 and LCDR1-3 comprising SEQ ID NOs:14-19, respectively; V_{H} and V_{L} comprising SEQ ID NOs:20 and 21, respectively; or SEQ ID NO:22 or 23. In certain embodiments, the anti-Bb antibody comprises HCDR1-3 and LCDR1-3 comprising SEQ ID NOs:4-9, respectively; V_{H} and V_{L} comprising SEQ ID NOs:10 and 11, respectively; or SEQ ID NO:12 or 13. In certain embodiments, the template AAV genome comprises SEQ ID NO:24, 25, 26, 27, 31, or 32.

In some embodiments, the host cell is a mammalian cell, optionally a 293, HeLa, or A549 cell.

In another aspect, the present disclosure provides a recombinant AAV composition produced by the method described herein. The present disclosure also provides a method of treating a disease in a human patient in need thereof, comprising delivering the recombinant AAV composition herein to the patient. Also provided herein are the recombinant AAV composition for use in treating a disease in a human patient in need thereof, and the use of the recombinant AAV composition for the manufacture of a medicament for treating a disease in a human patient thereof. In some embodiments, the disease to be treated is dry age-related macular degeneration (AMD).

Other features, objectives, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE FIGURES

FIG. 1A is a diagram illustrating that a shDNA-like sequence directs intra-molecular strand switching during replication, which leads to a U-turn back toward the wildtype ITR (wtITR) without synthesizing sequence beyond the hairpin structure. Newly synthesized DNA sequence is shown as a dotted line.

FIG. 1B is a diagram illustrating a normal AAV replication event that produces a full-length single-stranded AAV genome.

FIG. 1C is a diagram illustrating a working model for replication of vector genome with a short hairpin DNA (shDNA) like sequence. Step 1: DNA replication for AAV biosynthesis (starting from either ITR). Step 2: paused DNA polymerase due to secondary structure of a hairpin resembling ITR. Step 3: strand displacement (“snap-back” and elongation with nascent DNA as a template. Step 4: replication of the bidirectional construct in FIG. 1D (part 2) leads to generation of two self-complementary AAV (scAAV) vector genomes, one expressing anti-aC1s (αc1s) scFab and the other expressing an anti-Bb (αBb) scFab.

FIG. 1D is a diagram illustrating an exemplary bidirectional vector genome. The vector genome, shown in (1), contains a minCBA promoter that forms ITR-like secondary structures, shown in (2), due to palindromic sequences present in both the CMV enhancer and the CBA promoter. Replication of the vector genome leads to formation of two self-complementary vector genomes, shown in (3).

FIG. 1E is a diagram illustrating the design of an AAV vector genome (Diagram A) and the self-complementary vector genomes resulting from rAAV production in host cells (Diagrams B and C). Diagram A shows the structure of an AAV vector genome design with a bidirectional promoter driving expression of a first transgene (Transgene 1) and a second transgene (transgene 2). Both transgenes have a poly(A) signal sequence. The rAAV products generated during AAV production are a self-complementary Transgene 1 vector with two copies of the transgene 1 (Diagram B) and a self-complementary Transgene 2 vector with two copies of the Transgene 2 (Diagram C). The bidirectional minCBA promoter, due to the presence of shDNA-like sequences, acts as a pseudo ITR during replication to generate self-complementary AAV vectors.

FIG. 1F is a diagram illustrating two approaches to generate a full-length RNA transcript from two different rAAVs, each containing one of the two subparts (5′CDS and 3′CDS) of a full-length gene. The top approach uses an overlapping strategy, where the transgenes in the two AAV genomes contain overlapping sequences (indicated by the vertical dashed lines). The bottom approach uses a trans-splicing strategy involving a splice donor (SD) and a splice acceptor (SA). Prom: Promoter. CDS: coding sequence. PA: poly(A) signal sequence.

FIGS. 2A-2C show the annotated sequence (SEQ ID NO:1) of the minCBA promoter harboring palindromic sequences (or shDNA-like sequences) that behave like surrogate inverted repeat (IRS) elements promoting the formation of self-complementary AAV vector genomes during vector replication and production. Two pairs of IRS elements are present in the CMV enhancer (FIG. 2A and FIG. 2B, double-underlined), and two pairs in the CBA promoters (FIG. 2C, double-underlined).

FIG. 3 is a diagram illustrating the predicted folding configures (IR-1, IR-2, and IR-3) of the IRS elements described in FIGS. 2A-2C, respectively (Xie et al., Mol Ther. (2017) 25(6):1363-74).

FIG. 4A is a pair of diagrams illustrating two exemplary configurations (#9 and #10) of a construct harboring a bidirectional (“BiDir”) promoter (minCBA) driving expression of two independent antibody fragments (single-chain Fab or “scFab”). The first scFab is directed against activated complement subcomponent C1s, and the second scFab is directed against complement factor Bb. Unless otherwise specified herein, activated C1s is also referred to herein as “C1s.” Factor Bb is also referred to herein as “FBb” or simply “Bb.” Anti-C1s antibody fragment also is referred to herein as “αC1s.”

FIG. 4B is a pair of diagrams illustrating exemplary configurations of a construct harboring a bidirectional promoter driving expression of two independent antibody fragments that differ from constructs #9 and #10 by having charge mutations (#21 and #22) (“CM”; Δ) that are intended to promote cognate heavy chain and light chain pairing. In the figure, “Δ” indicates the presence of a charge mutation and is not meant to illustrate the exact positions or numbers of the charge mutations in the antibody fragment.

FIG. 5A is a diagram illustrating an exemplary bicistronic construct using a bidirectional promoter (a modified minCBA) that allows expression of two separate antibody fragments in opposite directions. BGH: bovine growth hormone.

FIG. 5B is a diagram illustrating the bidirectional minCBA promoter, which consists of two copies of the CBA promoter with an intervening enhancer sequence (sequences depicted in FIGS. 2A-2C) that can behave like an ITR or shDNA-like sequence because of the palindromic nature of its sequence.

FIG. 6 is panel of diagrams showing the design of an AAV vector genome (Diagram A) and the self-complementary vectors resulting from rAAV production in host cells (Diagrams B and C). Diagram A shows the structure of an AAV vector genome design with a bidirectional promoter driving expression of the anti-aC1s and anti-Bb scFab transgenes. Both transgenes have a BGH polyA. The rAAV products generated during AAV production are self-complementary aC1s vector with two copies of the anti-aC1s scFab transgene (Diagram B) and self-complementary Bb vector with two copies of the anti-Bb scFab transgene (Diagram C). The bidirectional minCBA promoter, due to the presence of shDNA-like sequences, acts as a pseudo ITR during replication to generate self-complementary AAV vectors.

FIGS. 7A-7C are diagrams illustrating the bidirectional CBA promoter driving anti-αC1s and anti-Bb transgenes. The configuration in FIG. 7A results in the packaging of two distinct self-complementary, snap-back vector genomes in a 1:1 ratio. The configurations in FIGS. 7B and 7C result in one kind of self-complementary, snap-back genome—anti-aC1s scFab encoding genome for FIG. 7B and anti-Bb scFab encoding genome for FIG. 7C.

FIGS. 8A and 8B are graphs showing AAV DNA analyzed under denaturing conditions in alkaline gel (FIG. 8B) or under non-alkaline conditions (FIG. 8A) in a TapeStation®. In FIG. 8A, the X-axis shows base-pair length, normalized to the two included ladders in each electropherogram, while the Y-axis captures relative fluorescence intensity. The size of the vector genome analyzed is about 2300 bp. FIG. 8B shows an alkaline agarose gel of the same extracted DNA as in FIG. 8A. Alkaline gel analysis: Lane 1: standard ladder; Lane 2: PCL-ESB02.

FIGS. 9A and 9B are graphs showing extracted rAAV DNA sizing for rAAV produced in two triple transient transfection (TTx) experiments (FIG. 9A) and in four PCL production experiments (FIG. 9B). Sizing of DNA was performed with the Agilent 4200 TapeStation® and D5000 ScreenTape assay. The X-axis shows base-pair length, normalized to the two included ladders in each electropherogram, while the Y-axis captures relative fluorescence intensity.

FIGS. 10A and 10B are graphs showing the long-read sequencing read length distributions from the four PCL (FIG. 10A) and two TTx (FIG. 10B) generated vector lots. FIG. 10A shows the read length distribution for rAAV in four PCL-produced lots, CER1, CER2, ESB2, WRS R23258. FIG. 10B shows data from two TTx-produced lots, X22122A and VP101622 lots. The X-axis captures the distribution of read length size (in bp) while the Y-axis indicates the number of reads per detected size.

FIG. 11 is a diagram showing a visual representation of a long-read sequencing DNA library from a self-complementary rAAV vector. The delta-ITR region (regions directly under the 5′ designation) represents a closed end inaccessible for the ligation of the sequencing adapter (loop on the right). Sequencing adapters can only ligate to the single, open end of the rAAV vector, attaching to 3′ end of the molecule at the sense (bottom) and anti-sense (top) ITR junction.

FIG. 12A is a bar graph showing anti-aC1s and anti-Bb vector genome levels in the mouse retina qPCR quantification of vector genome levels in the mouse retina from triple transfection (TTX-VP101622) and stable producer cell line (PCL-ESB02) transduced eyes. Data show the median+MAD (median absolute deviation). Dose-response P values indicate trend of increasing vector genome with dose level via exact one-sided Jonckheere-Terpstra trend test.

FIG. 12B is a pair of bar graphs showing RT-qPCR quantification of anti-aC1s and anti-Bb scFab transcripts in the mouse retina from TTX-VP101622 and PCL-ESB02 transduced eyes. Data show the median+MAD. Dose-response P values indicate trend of increasing transcript copies with dose level via exact one-sided Jonckheere-Terpstra trend test

FIG. 12C is a line graph showing the correlation between anti-aC1s and anti-Bb transcript levels across all samples, both TTx- and PCL-generated material. Spearman r>0.99.

FIGS. 13A and 13B are vector genome schematics showing dual transgene expression: one transgene is driven by a CBA promoter and the other by a synapsin promoter. Both promoters are regulated by a CMV enhancer situated between them. This configuration predominantly yields full-length packaged genomes (˜4.8 kb).

FIGS. 14A and 14B are graphs showing analytical ultracentrifugation (AUC) analysis of vector preps VP031125 MB (FIG. 14A) and VP031125BB (FIG. 14B), demonstrating that the vector preparations are enriched for full vector genomes as represented by a species sedimenting at an S value consistent with genome containing particles. There is no evidence of empty capsids.

FIGS. 15A-15C represent sequencing data showing the presence of full-length vector genomes for VP031125BB, consistent with the configuration in FIGS. 13A and 13B.

FIGS. 16A-16C represent sequencing data showing the presence of full-length vector genomes for VP031125 MB, consistent with the configuration in FIGS. 13A and 13B.

FIG. 17 is a bar graph showing aC1s and Bb scFab transcript driven by the CBA or human synapsin (hSyn) promoter in rAAV-infected 293 cells.

FIG. 18 is a schematic showing an ITR plasmid design that promotes the formation of both dimeric self-complementary and monomeric species.

FIGS. 19A-19D are schematics showing self-complementary AAV vector genome designs. FIGS. 19A and 19C shows a vector design for dual expression of anti-aC1s scFab. FIGS. 19B and 19D show a vector design for dual expression of anti-Bb scFab. The vectors utilize a bidirectional minCBA promoter, and inverted repeat sequences in the promoter facilitate double-stranded AAV vector formation. The vectors also comprise a BGH polyA sequence, a CMV enhancer, and 5′ and 3′ ITRs.

FIG. 20 is a graph showing AUC analysis of AAV2 self-complementary EGFP vector.

FIGS. 21A and 21B are graphs showing AUC analysis of AAV.SAN024 harboring dimeric self-complementary anti-aC1s scFab vector genome (FIG. 21A) and dimeric self-complementary anti-Bb scFab vector genome (FIG. 21B).

FIGS. 22A-22C are graphs showing summaries of the long-read sequencing results for an aC1s scAAV lot (see FIG. 19C). FIG. 22A shows the size profile of extracted rAAV DNA under non-denatured conditions. FIG. 22B shows sequencing read length (bp) distributions. FIG. 22C shows the transgene configuration patterns detected by BLASTN analysis specific to the expected full-length rAAV transgene, including detected number, mean base pair length, and the relative percentage.

FIG. 23A-23C are graphs showing summaries of the long-read sequencing results for a Bb scAAV lot (see FIG. 19D). FIG. 23A shows the size profile of extracted rAAV DNA, under non-denatured conditions. FIG. 23B shows sequencing read length (bp) distributions. FIG. 23C shows the transgene configuration patterns detected by BLASTN analysis specific to the expected full-length rAAV transgene, including detected number, mean base pair length, and the relative percentage.

### DETAILED DESCRIPTION OF THE INVENTION

The present invention is based on the discovery that a recombinant AAV genome having a bicistronic, bidirectional expression cassette for two transgenes can lead to generation of two rAAV virions (dual populations), one with a self-complementary genome carrying an expression cassette for one transgene and the other with a self-complementary genome carrying an expression cassette for the other transgene. This phenomenon occurs when the bicistronic, bidirectional expression cassette comprises a bidirectional promoter that can form a palindromic structure due to the presence of inverted repeat sequences within the promoter.

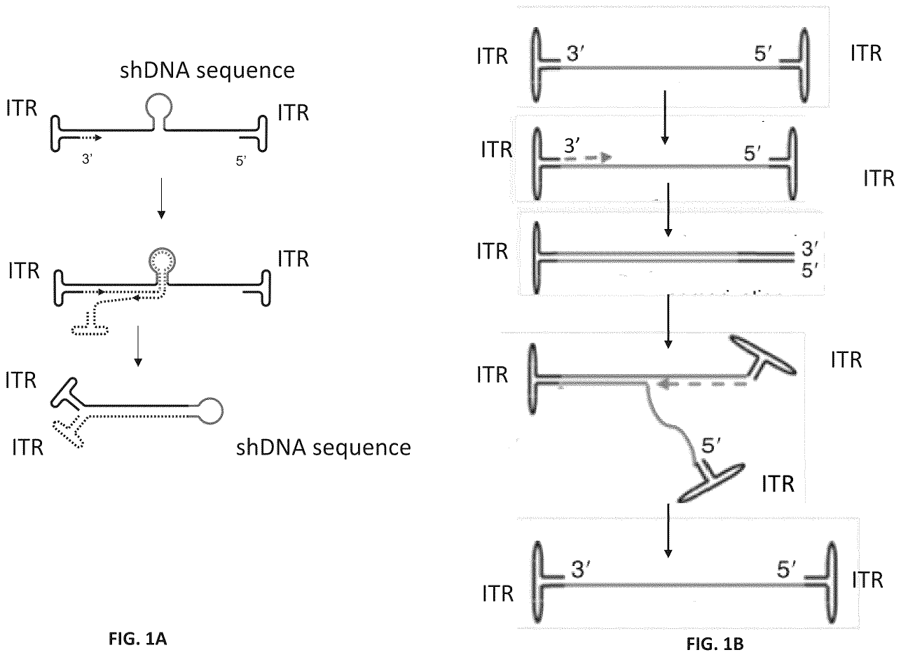

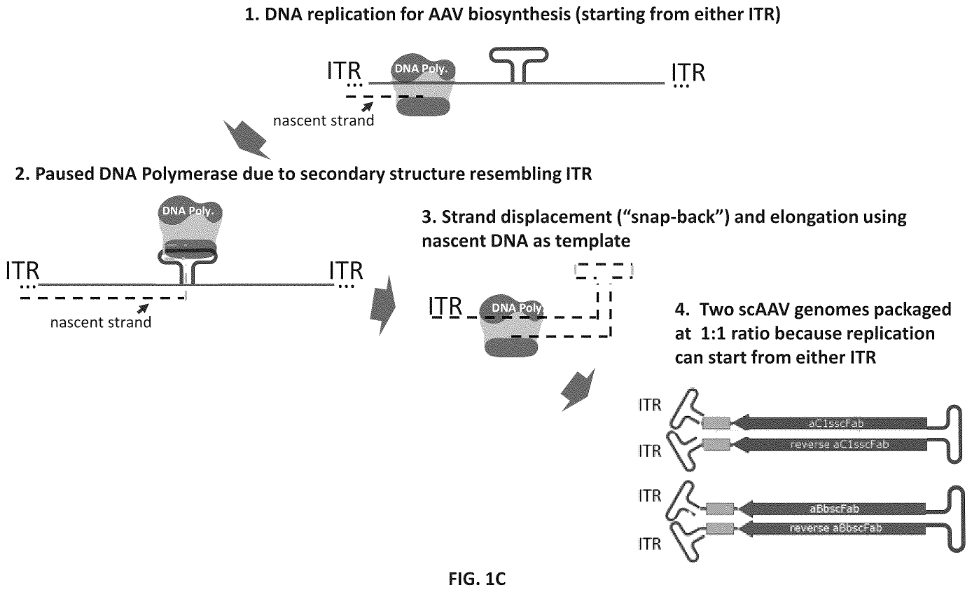

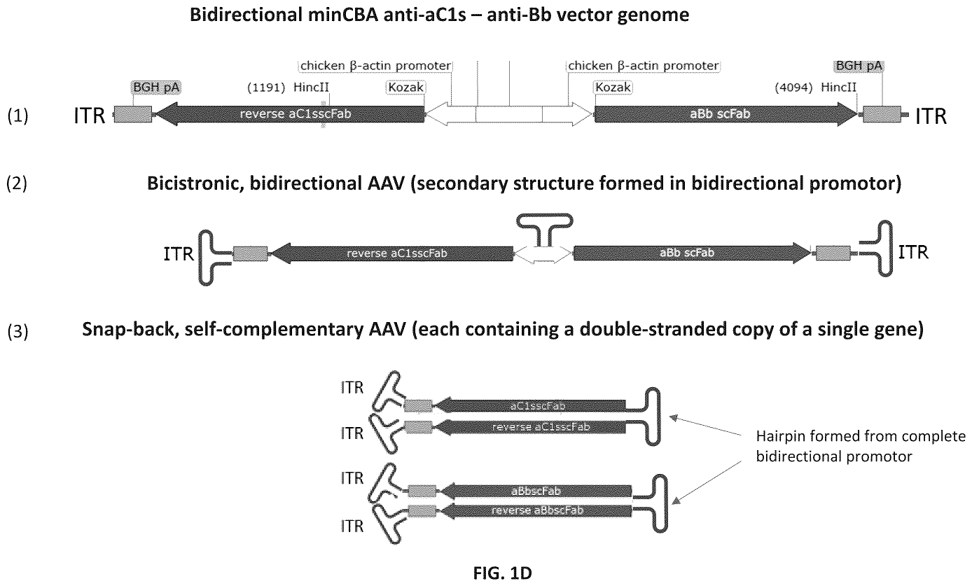

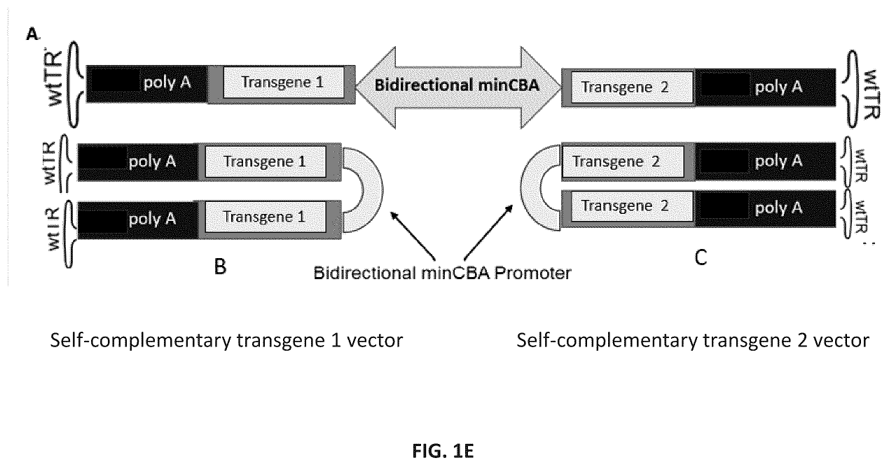

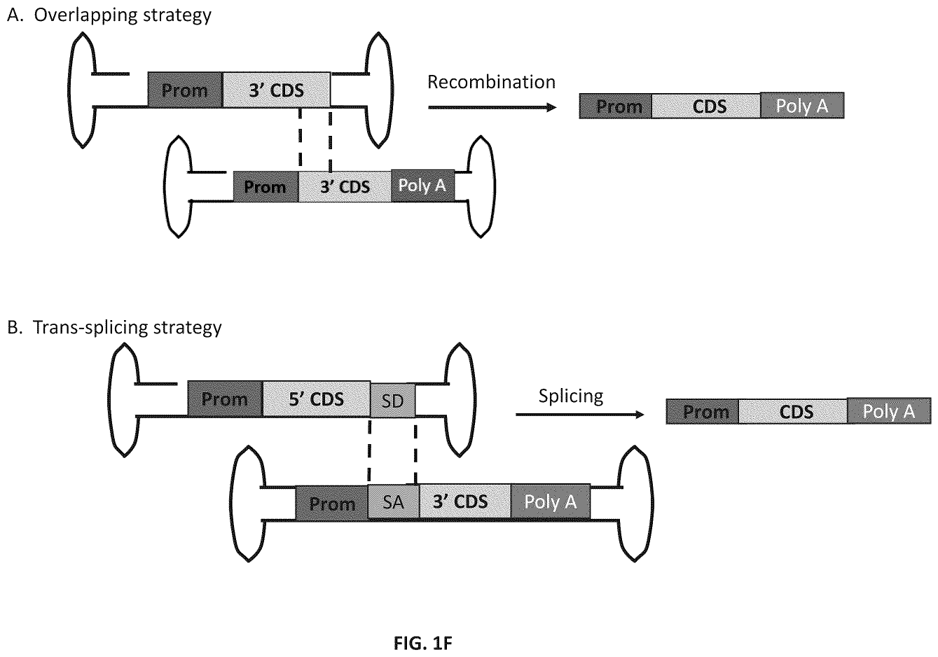

Without being bound by theory, the inventors believe that during DNA replication, the palindromic conformation formed by the bidirectional promoter leads to a “U-turn” of the DNA polymerase on the nascent DNA strand, where the DNA polymerase continues DNA synthesize using that nascent DNA strand as a template, resulting in a self-complementary vector genome (FIGS. 1A and 1C). Without a U-turn, the DNA polymerase proceeds to replicate the entire vector genome, generating a single-stranded vector genome with the entire expression cassette of the original design (FIG. 1B).

Accordingly, the present disclosure provides a method for producing an rAAV composition with at least two rAAV populations from just one cell (FIGS. 1D and 1E). In some embodiments, the present disclosure provides a method for producing an rAAV composition with dual rAAV populations from just one cell. In some embodiments, the rAAV composition additionally has an rAAV population containing the vector genome of the original design (i.e., carrying two transgenes).

In some embodiments, the two transgenes are the same, and the method herein leads to the generation of an rAAV population with a self-complementary vector genome.

The rAAV composition herein can be used to concurrently deliver two separate therapeutic proteins to a patient. Typically, in order to produce two different viral vectors, two different cells are used with each engineered to produce one of the two viral vectors. For example, to produce two different AAV vectors, two separate PCLs are established, each cell line producing one of the two vectors. The present method streamlines this process by using just one cell (e.g., one PCL), greatly reducing the costs for AAV production, especially for gene therapy that requires the delivery of two different proteins.

The present disclosure also provides a method of introducing a protein encoded by a large transgene into a target cell through a viral vector whose genome capacity is smaller than the size of the transgene. In this method, the large transgene is split into two parts, each part being one of the transgenes in the aforementioned bicistronic, bidirectional expression cassette. The AAV production method herein is then used to produce an rAAV population with one rAAV for expressing one part of the large transgene and another rAAV for expressing the second part of the large transgene. When this mixed AAV population is introduced into the same target cell, separate transcripts from the two rAAVs can be spliced together to form a full-length transcript for the large transgene (FIG. 1F, “trans-splicing strategy”). Alternatively, the two input DNA vector genomes can recombine through homologous recombination to generate a full-length DNA sequence encoding the entire protein (FIG. 1F, “overlapping strategy”). This use of the present discovery thus overcomes the size limitation of rAAV technology.

It is advantageous to produce two populations of AAV vectors using one cell (e.g., one PCL) over using two different cells (e.g., two PCLs). For example, when using two PCLs, each PCL would require an independent production and characterization campaign. Moreover, the production runs from each cell line would require individual characterization and separate Drug Product release criteria. In this invention, only one Drug Product run is necessary with one vector lot release. This has considerable cost and time savings. Further, self-complementary vectors, being already double-stranded, do not have the limitation of requiring second strand synthesis, unlike single-stranded AAV vectors. Consequently, onset of gene expression from self-complementary (double-stranded) AAV vectors is quicker than from single-stranded AAV vectors. Additionally, not all single-stranded AAV vectors generate a second strand, so there is a loss of vector genomes and potency; this loss is less likely to occur with self-complementary vectors.

Traditionally, scAAV production relies on a mutated inverted terminal repeat (ΔITR) strategy to promote double-stranded genome formation. However, this approach is inherently imperfect, as terminal resolution events can still occur during vector replication and packaging. Such events result in the formation of undesired single-stranded monomeric genomes, approximately 2.3 kilobases in length, which compromise vector homogeneity and may reduce therapeutic potency. The present invention overcomes these limitations by employing a bidirectional promoter (e.g., minCBA) in place of the conventional ΔITR-based design. This configuration enables the production of greater than 70% double-stranded vectors while markedly reducing the prevalence of monomeric species. The structural integrity and improved uniformity of the resulting vectors have been confirmed through analytical ultracentrifugation (AUC) and PacBio® single-molecule real-time sequencing, validating the robustness and efficiency of this approach. By utilizing the bidirectional promoter's inverted repeat sequences to facilitate snap-back formation, the present method is more efficient and reliable than the traditional mutated ITR approach in producing self-complementary AAV genomes.

### I. AAV Genome Template

The present rAAV production method entails introducing into a host cell, stably or transiently, a template for an AAV vector genome comprising a bicistronic expression cassette with at least one bidirectional promoter (e.g., one bidirectional promoter in the middle, or two bidirectional promoters), where the promoter contains palindromic sequences. When AAV replicases, capsid proteins, and helper components are present, the template will direct the replication and packaging of rAAV virions. The AAV vector genome is further described in detail below. In some embodiments, the DNA template carries two or more bicistronic, bidirectional expression cassettes.

### A. Bicistronic, Bidirectional Expression Cassette

In some embodiments, the bicistronic expression cassette described herein comprises two transgenes placed in opposite directions and separated by a bidirectional promoter linked operably to the two transgenes. In some embodiments, the bidirectional promoter used herein may be G/C-rich; that is, more than 50% of the nucleotides in the promoter are G and C nucleotides. The bidirectional promoter contains one or more (e.g., one, two, three, or more) pairs of inverted repeats such that each pair of the inverted repeats can hybridize to each other, leading to formation of a palindromic secondary structure in the promoter. In some embodiments, the inverted repeats may be 10 or more nucleotides (e.g., 10 to 30 nucleotides) in length. For example, the inverted repeats may be 11, 12, 13, 14, 15, 16, 17, 18, 19, 20, 21, 22, 23, 24, or 25 nucleotides in length.

In some embodiments, the bidirectional promoter comprises two copies of a unidirectional promoter placed in opposite directions. The unidirectional promoter may be, for example, a chicken β-actin (CBA) promoter, a synapsin promoter, an EF1α promoter, a human ubiquitin C promoter, a GRK1 promoter, a rhodopsin promoter, or a PGK promoter. In further embodiments, the unidirectional promoter is a CBA promoter comprising SEQ ID NO:2 or a nucleotide sequence that is at least 85% (e.g., at least 86, 87, 88, 89, 90, 91, 92, 93, 94, 95, 96, 97, 98, or 99%) identical thereto.

In some embodiments, the two copies of unidirectional promoters are separated by a nucleotide linker. In further embodiments, the nucleotide linker may comprise an enhancer, such as a CMV enhancer. In certain embodiments, the CMV enhancer comprises SEQ ID NO:3 or a nucleotide sequence that is at least 85% (e.g., at least 86, 87, 88, 89, 90, 91, 92, 93, 94, 95, 96, 97, 98, or 99%) identical thereto.

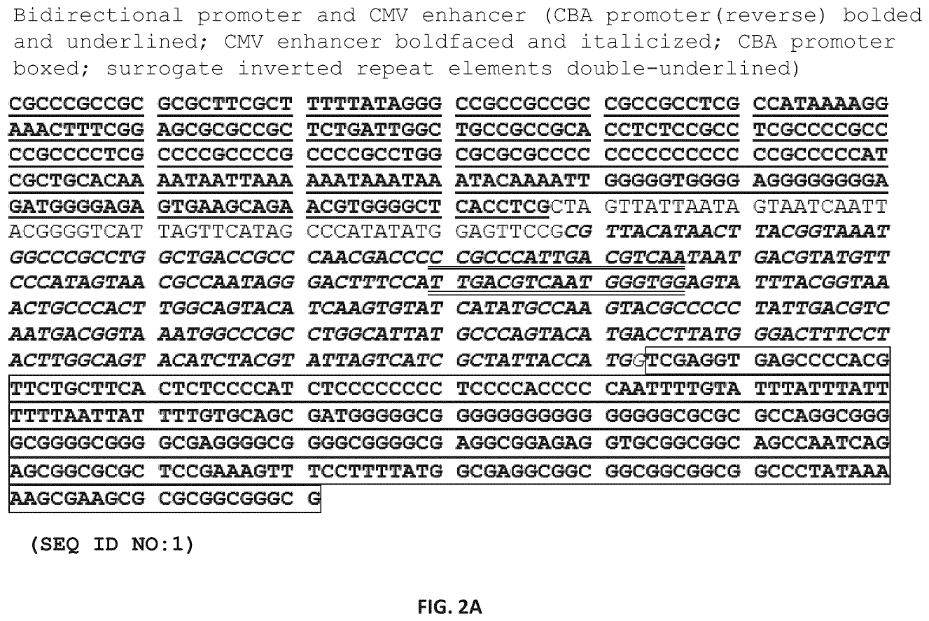

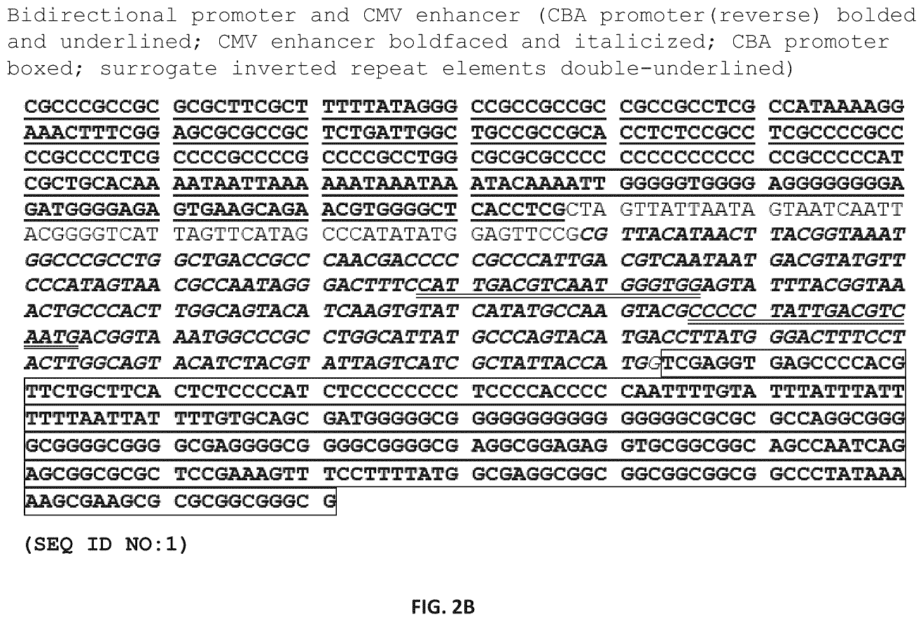

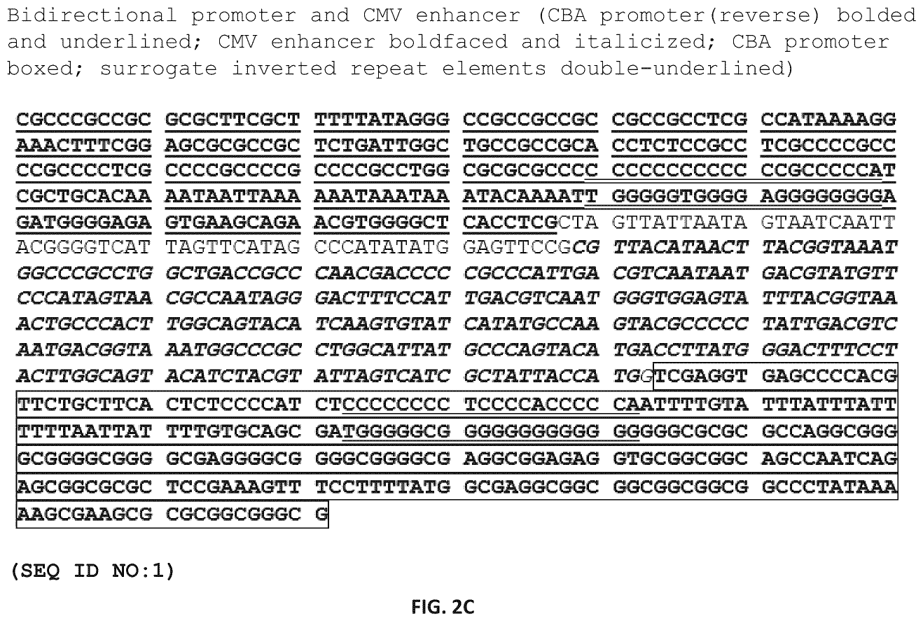

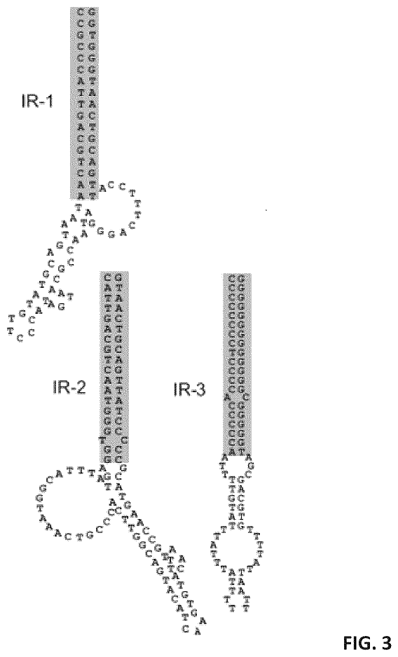

In some embodiments, the bidirectional promoter comprises a minimal CBA promoter and a CMV enhancer. In further embodiments, the bidirectional promoter comprises SEQ ID NO:1 or a nucleotide sequence that is at least 85% (e.g., at least 86, 87, 88, 89, 90, 91, 92, 93, 94, 95, 96, 97, 98, or 99%) identical thereto. As shown in FIGS. 2A-2C, the bidirectional promoter comprising SEQ ID NO:1 have four pairs of inverted repeats (double-underlined)—two in the CMV enhancer (FIGS. 2A and 2B), and two in the promoter region (FIG. 2C). These four pairs of inverted repeats can generate three palindromic configurations in the bidirectional promoter (FIG. 3).

In some embodiments, each transgene in the bicistronic expression cassette has a poly(A) signal sequence at its 3′ end (in its transcription direction). The poly(A) signal may be derived from any mammalian gene. For example, the poly(A) signal may be from a bovine growth hormone (BGH) gene, e.g., comprising SEQ ID NO:28 or a nucleotide sequence that is at least 85% (e.g., at least 86, 87, 88, 89, 90, 91, 92, 93, 94, 95, 96, 97, 98, or 99%) identical thereto.

The bicistronic, bidirectional expression cassette may also contain additional transcriptional regulatory elements. For example, each transgene may contain a Kozak sequence and a sequence that enhances gene expression or RNA stability (e.g., a WPRE element). The transgenes also may contain an intron sequence such as a chimeric intron that helps to increase transgene expression levels by promoting transport of mRNA out of the nucleus and enhancing mRNA stability. For example, the bidirectional promoter may comprise SEQ ID NO:30 or a nucleotide sequence that is at least 85% (e.g., at least 86, 87, 88, 89, 90, 91, 92, 93, 94, 95, 96, 97, 98, or 99%) identical thereto.

In the AAV vector genome, the bicistronic, bidirectional expression cassette is flanked by a pair of AAV ITRs. The AAV ITRs may be derived from the same or from different AAVs. In some embodiments, the bidirectional expression cassette is flanked by AAV ITRs from the same AAV, such as AAV2 ITRs.

### B. Transgene Coding Sequences

The transgenes in the bicistronic, bidirectional expression cassette herein may be codon-optimized, if desired, to improve their expression in human cells. The coding sequences may encode a signal peptide (e.g., a signal peptide from IgG Kappa) to support secretion of the proteins.

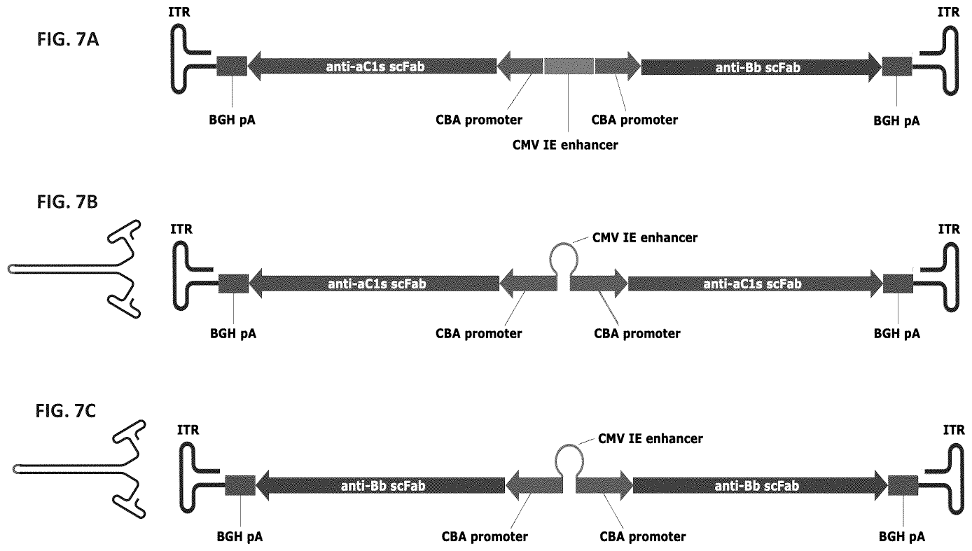

In some embodiments, the transgenes in the bicistronic, bidirectional expression cassette encode two different therapeutic proteins (see, e.g., FIG. 7A). In some embodiments, the bicistronic, bidirectional expression cassette encodes two different therapeutic proteins for treating the same disease. For example, one protein targets a biological pathway in one disease and the other protein targets a complementary pathway involved in that disease, such that efficacy of treatment is enhanced, e.g., additively or synergistically. Producer cells comprising this expression cassette will produce an rAAV composition comprising two rAAV species, one for each therapeutic protein. See section III below for non-limiting examples. In some embodiments, the two transgenes in the bicistronic, bidirectional expression cassette encode the same therapeutic proteins (see, e.g., FIGS. 7B and 7C).

In some embodiments, the transgenes in the bicistronic, bidirectional expression cassette encode two parts of a single therapeutic gene. In some embodiments, the single therapeutic gene is larger than the genome capacity of AAV. For example, the therapeutic gene is between about 4.7 kb and about 9.4 kb in size. The first transgene (“upstream transgene”) contains the 5′ part of the therapeutic gene and the second transgene (“downstream transgene”) contains the 3′ part of the therapeutic gene, each part not exceeding the 4.7 kb AAV genome size limit. In an overlapping strategy, the 3′ coding region of the upstream transgene overlaps with the 5′ coding region of the downstream transgene by, for example, 10 or more nucleotides (e.g., 10 to 200 nucleotides); these two partial transgenes can hybridize to each other via intermolecular recombination at the overlapping region and form through homologous recombination a full-length transgene (FIG. 1F, top). In a trans-splicing approach, the 3′ end of the coding region of the upstream transgene comprises a splice donor and the 5′ end of the coding region of the downstream transgene comprises a splice acceptor; when both transcripts are made in the same cell, the two transcripts are spliced into one full-length transcript of the therapeutic gene through splice donor/acceptor interaction (FIG. 1F, bottom). The upstream transgene does not contain a poly(A) signal sequence, while the downstream transgene does. Producer cells comprising this expression cassette will produce an rAAV composition comprising two rAAV species, one containing the upstream transgene and the other containing the downstream transgene; upon co-transduction of the target cell with the mixed rAAV composition, reconstitution of a full-length transcript can occur.

In some embodiments, the two transgenes in the expression cassette are identical, and the cell comprising the expression cassette produces a population of rAAV comprising a self-complementary vector genome. See, e.g., FIGS. 7B and 7C.

### II. Production of rAAV Compositions

The present rAAV production method allows the production of a mixed population of two different rAAVs in a single cell, e.g., when the rAAV genomic template contains two different transgenes placed in opposite orientations and directed by a common bidirectional promoter. The present method also has the advantage of producing self-complementary rAAV genomes, whether the two transgenes are different or the same.

The cell may be a human cell such as an HEK293 cell, an HEK293T cell, a HeLa cells, or an A549 cell. The cell may be an insect cell such as a Spodoptera frugiperda (e.g., sf9) cell.

### A. AAV Serotype

Depending on the AAV Cap gene introduced into the host cell, rAAV of any serotype can be produced. In some embodiments, the rAAVs produced herein may be of AAV1, AAV2, AAV3, AAV3B, AAV4, AAV5, AAV6, AAV7, AAV8, AAV8.2, AAV9, or AAVrh10 serotype, or of a pseudotype or a serotype that is a mutant, hybrid, variant or derivative of one of the AAV serotypes listed herein (i.e., AAV derived from multiple serotypes). The Cap gene may be engineered such that the capsid proteins encoded by it have reduced immunogenicity or enhanced transduction ability in humans or nonhuman primates.

In some embodiments, the rAAV is of a serotype with tropism for the eye. In some embodiments, the rAAV is of AAV2.7m8, AAV5, AAV8, AAV9, AAVR100, AAVPHP.eB, AAVPHP.B, AAVrh78R, or AAV.ANC80 serotype. In certain embodiments, the rAAV is of AAV2 or AAV5 serotype. In some embodiments, the rAAV has a wildtype capsid, such as a capsid of wildtype AAV2 or AAV5 serotype. In further embodiments, the rAAV has a wildtype AAV2 capsid and its recombinant genome comprises AAV2 inverted terminal repeats (ITR) flanking the payload nucleotide sequence.

In some embodiments, the rAAV is of a serotype with tropism for the liver (e.g., AAV3 or AAV5), muscle (e.g., AAV8, AAV9, or AAVrh.74), brain (e.g., AAV9 or AAVrh.10), or lung (e.g., AAV2).

In some embodiments, the rAAV herein has an AAV2 capsid. In particular embodiments, the AAV2 capsid is a wildtype AAV2 capsid. In other embodiments, the AAV2 capsid contains mutations that improve the rAAV2's potency and production yield.

B. rAAV Production by PCL

In some embodiments, the present rAAV production method uses a PCL (e.g., a human PCL) that has stably integrated into its genome a template for an AAV vector genome comprising a bicistronic, directional expression cassette described herein, an AAV Rep gene, and an AAV Cap gene.

Production of rAAV may be initiated by infecting the PCL with a helper virus such as an adenovirus or a herpes simplex 1 virus (HSV1). If desired, the helper virus may be replication-deficient. If the helper virus is replication-competent, the helper virus may be removed during downstream processing. Alternatively, helper genes (e.g., adenovirus adenovirus E4, E2a, and VA genes) may be stably integrated into the PCL as well and placed under transcriptional control of inducible promoters; production of rAAV may be initiated by turning on the inducible promoters using, e.g., a small molecule.

In some embodiments, the PCL may have one or more of the following characteristics: (i) the integrated exogenous DNA sequences remain stable over passaging without drug selection; (ii) before rAAV production, the AAV promoters (e.g., p40, p5, and p19) remain silent during cultivation and cell amplification; and (iii) upon infection with a helper virus, elevated expression and amplification of Rep/Cap is induced, and the rAAV genome is rescued and replicated. See, e.g., Merten et al., Microorganisms (2024) 12:384.

C. rAAV Production by Transient Transfection

In some embodiments, the present rAAV production method uses a human host cell transiently transfected with one or more DNA vectors carrying the rAAV vector genome template, the AAV Rep and Cap genes, and helper components such as adenovirus E4, E2a, and VA genes. In some embodiments, three DNA vectors are used: (1) a plasmid encoding the rAAV vector genome, (2) an AAV helper plasmid encoding the AAV replicases and capsid proteins, and (3) a pAd helper plasmid encoding the E4, E2a and VA genes.

D. Purification of rAAV

Once desired rAAV yields are achieved, supernatants from the cell culture are harvested and purified using methods known in the art. The relative vector genome amounts of each rAAV species may be determined by well-known methods such as quantitative PCR and the sequencing technology disclosed below in the Examples.

### III. Pharmaceutical Use of AAV Compositions

Recombinant AAVs produced herein may be formulated into pharmaceutical compositions. The pharmaceutical compositions may comprise pharmacologically acceptable carriers, diluents, and/or excipients. For example, the compositions may comprise a tonicity agent (e.g., sodium chloride, amino acids, sugars, or combinations thereof), a surfactant (e.g., polysorbate 20 or polysorbate 80), and/or a stabilizer (e.g., a methionine).

The pharmaceutical compositions may be delivered in vivo to a desired tissue such as the lungs, liver, muscle, brain, or the eye. Depending on the target tissue, the compositions may be delivered to a patient by subcutaneous, oral, subcutaneous, intra-muscular, or intravenous route. In the case of the eye, the compositions may be delivered by sub-retinal or intravitreal injection (e.g., front, mid or back vitreous injection).

The pharmaceutical compositions may be delivered in a therapeutically effective amount to treat a disease, including a congenital disease. A “therapeutically effective amount” means a dosage sufficient to produce a desired result, e.g., amelioration of one or more symptoms of the disease to be treated. Examples of diseases are described elsewhere herein.

The patient may be treated, before, during, and/or after the rAAV injection, with an anti-inflammatory agent (e.g., a corticosteroid) to prevent or ameliorate potential immune response against the rAAV. The anti-inflammatory agent may be, for example, a methylprednisolone, difluprednate, triamcinolone, dexamethasone, etc. The anti-inflammatory agent may be administered locally (e.g., through eye drops) or systematically. In some embodiments, the agent may be administered locally, or at or near the AAV injection site. To prevent anti-drug antibody (ADA) response, the patient may be pre-treated with an IgG-degrading enzyme to reduce levels of preexisting neutralizing antibodies to AAV prior to AAV administration.

Non-limiting examples of diseases that can be treated with the mixed AAV compositions herein are described below.

### A. AAV-Based Gene Therapy for Dry AMD

In some embodiments, the two transgenes in the bicistronic, bidirectional expression cassette encode an antibody fragment that inhibits the classical complement pathway and an antibody fragment that inhibits the alternative complement pathway. An example of the former is an anti-aC1s antibody fragment (e.g., scFv or scFab). An example of the latter is an anti-Bb antibody fragment (e.g., scFv or scFab).

In some embodiments, the anti-aC1s antibody fragment herein comprises heavy chain complementarity-determine regions (HCDR) 1-3 comprising SEQ ID NOs:4-6, respectively, and light chain CDR (LCDR) 1-3 comprising SEQ ID NOs:7-9, respectively. In further embodiments, the anti-aC1s antibody fragment comprises a V_{H} comprising SEQ ID NO:10 or an amino acid sequence at least 95% (e.g., at least 96, 97, 98, or 99%) identical thereto; and a V_{L} comprising SEQ ID NO:11 or an amino acid sequence at least 95% (e.g., at least 96, 97, 98, or 99%) identical thereto. In certain embodiments, the anti-aC1s antibody fragment comprises a peptide linker, such as a flexible linker, e.g., a linker comprising (G₄S)ₙ (SEQ ID NO:29), where n=1, 2, 3, 4, 5, 6, 7, 8, 9, or 10, linking the V_{H} and the V_{L} of the antibody fragment. In particular embodiments, the anti-aC1s scFab herein comprises SEQ ID NO:12 or 13, or an amino acid sequence at least 95% (e.g., at least 96, 97, 98, or 99%) identical thereto.

In some embodiments, the anti-Bb antibody fragment herein comprise HCDR1-3 comprising SEQ ID NOs:14-16, respectively, and LCDR1-3 comprising SEQ ID NOs:17-19, respectively. In further embodiments, the anti-Bb antibody fragment comprises a V_{H} comprising SEQ ID NO:20 or an amino acid sequence at least 95% (e.g., at least 96, 97, 98, or 99%) identical thereto; and a V_{L} comprising SEQ ID NO:21 or an amino acid sequence at least 95% (e.g., at least 96, 97, 98, or 99%) identical thereto. In certain embodiments, the anti-Bb antibody fragment comprises a peptide linker, such as a flexible linker, e.g., a linker comprising (G₄S)ₙ (SEQ ID NO:29), where n=1, 2, 3, 4, 5, 6, 7, 8, 9, or 10, linking the V_{H} and the V_{L} of the antibody fragment. In particular embodiments, the anti-Bb scFab herein comprises SEQ ID NO:22 or 23, or an amino acid sequence at least 95% (e.g., at least 96, 97, 98, or 99%) identical thereto.

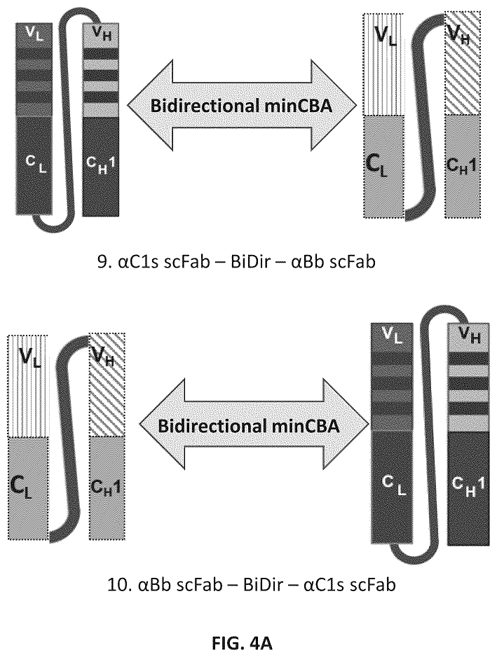

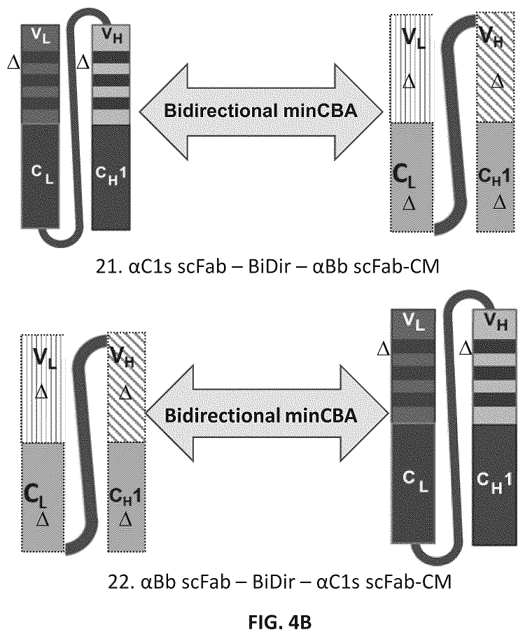

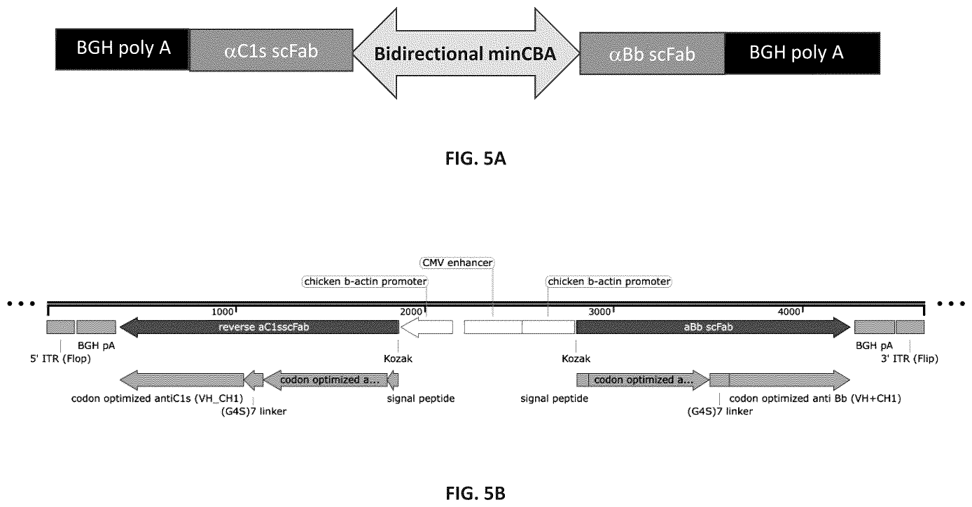

Examples of bicistronic, bidirectional expression cassettes comprising transgenes encoding an anti-aC1s scFab and an anti-Bb scFab are constructs #9, #10, #21, and #22 shown in FIGS. 4A and 4B and listed below (BiDir: bidirectional promoter):
#9: αC1s scFab-BiDir-αBb scFab (SEQ ID NO:24)
#10: αBb scFab-BiDir-αC1s scFab (SEQ ID NO:25)
#21: αC1s scFab-BiDir-αBb scFab-CM (SEQ ID NO:26)
#22: αBb scFab-BiDir-αC1s scFab-CM (SEQ ID NO:27)
   Further details of construct #9 are illustrated in FIGS. 5A and 5B.

In constructs #21 and #22, both the anti-C1s and anti-Bb scFabs contain charge mutations (CMs) to promote cognate pairing of the heavy and light chains within each antibody fragment. To generate charge mutants (CM), specific amino acids were substituted in the variable and/or constant domains of the αC1s and αBb antibody fragments. The following amino acid changes were introduced for the following mutated antibody fragments:
αC1s scFab-CM: Q42E and Q292K (numbering in accordance with SEQ ID NO:13);
αBb scFab-CM: Q38K, S114A, N137K, Q288E, and T434E (numbering in accordance with SEQ ID NO:23)

In some embodiments, the host cell contains a template for an rAAV vector genome comprising SEQ ID NO:24, 25, 26, OR 27; a nucleotide sequence encoding the same amino acid sequences as does SEQ ID NO:24, 25, 26, or 27; or a nucleotide sequence that is at least 85% (e.g., at least 90, 91, 92, 93, 94, 95, 96, 97, 98 or 99%) identical to SEQ ID NO:24, 25, 26, or 27.

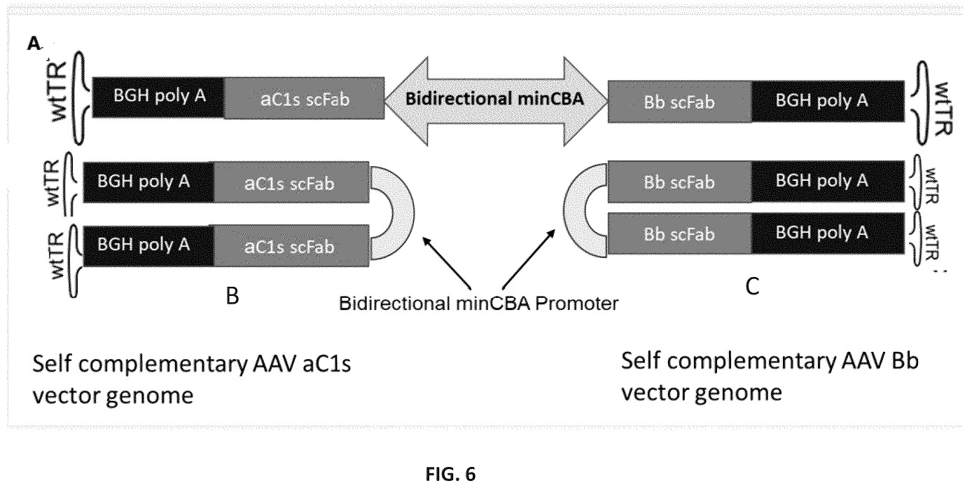

By way of example, a host cell harboring AAV2 #9 (SEQ ID NO:24) can produce a mixed rAAV population with two rAAV species: one species is a self-complementary rAAV comprising a transgene encoding the anti-aC1s scFab, and the other species is a self-complementary rAAV comprising a transgene encoding the anti-Bb scFab (FIG. 6).

The mixed rAAV populations comprising rAAVs for expressing an anti-aC1s antibody fragment and an anti-Bb antibody fragments, such as the mixed rAAV populations produced by host cells comprising construct #9, #10, #21, or #22, can be used to treat dry AMD. A pharmaceutical comprising the mixed rAAV populations may be injected intravitreally to a diseased eye at a therapeutically effective dosage. For example, the rAAV may be delivered to the eye at a dose of about 1E7 to about 1E15 vector genomes (VG), for example, about 1E10 to about 1E12 VG, per eye. In some embodiments, the dosage of rAAV injected into the eye is 1E8 to 1E14, 10E9 to 10E13, 10E9 to 10E12 VG. The therapeutic effectively amount ameliorates one or more symptoms of dry AMD, such as growth of GA lesions, retinal lesions, destruction of retinal layer, or photoreceptor cell death, and/or slowing progression of the disease. A desired result may also include improvement in one or more functional symptoms; for example, the desired result may be reduction of visual distortions, improved central vision, improved vision in low light settings, and/or reduced blurriness. By “treat” is meant amelioration of one or more symptoms of the disease and/or slowing of the progress of the disease.

In some embodiments, the diseased may be pre-treated with an intravitreal injection of an IgG-degrading enzyme to reduce potential ADA. The patient may also be treated with a corticosteroid as described above.

In some embodiments, the rAAV herein has an AAV2 capsid. In particular embodiments, the AAV2 capsid is a wildtype AAV2 capsid. In other embodiments, the AAV2 capsid contains mutations that improve the rAAV2's potency and production yield.

### B. AAV-Based Gene Therapy for Additional Diseases

The present method may be used to generate dual-targeting rAAV compositions to treat additional diseases. For example, the compositions may comprise rAAVs encoding therapeutic proteins (e.g., antibodies or antigen-binding fragments thereof) that target VEGF and IL-6, or target VEGF and a complement factor, to treat wet AMD.

The present method also may be used to generate a dual population of self-complementary AAVs that each carry a portion of a large therapeutic gene. As described above, when introduced into a target cell in vivo, transcripts from the two AAVs can reconstitute a full-length transcript of the gene, allowing expression of a therapeutic protein encoded by the gene.

By way of example, the therapeutic gene is an ABCA4 (ATP-binding cassette, sub-family A, member 4) gene, and the rAAV composition can be used to treat Stargardt disease, an autosomal recessive disease that causes macular degeneration. The ABCA4 gene is expressed in outer segment disk edges of rod photoreceptors and its cDNA is about 7 kb in length. The gene can be split into two halves of about 3.5 kb each, with each half carried by a separate rAAV.

In another example, the therapeutic gene is a MYO7A gene, which encodes myosin VIIa and may be used to treat Usher Syndrome or retinitis pigmentosa. In yet another example, the therapeutic gene is a OTOF gene, which encodes otoferlin and may be used to treat deafness caused by OTOF mutations.

### IV. Exemplary Embodiments

Further particular embodiments of the present disclosure are described as follows. These embodiments are intended to illustrate the compositions and methods described in the present disclosure and are not intended to limit the scope of the present disclosure.

1. A method of producing a recombinant adeno-associated virus (AAV) composition comprising a first AAV having a self-complementary recombinant genome comprising a first transgene and a second AAV having a self-complementary recombinant genome comprising a second transgene, the method comprising: introducing into a host cell an exogenous DNA encoding a template AAV genome comprising an expression cassette flanked by AAV inverted terminal repeats (ITRs), wherein the expression cassette comprises the first and second transgenes placed in opposite directions and separated by a bidirectional promoter, and wherein the bidirectional promoter comprises palindromic sequences, culturing the host cell under conditions for AAV replication, wherein a recombinant AAV composition is produced, and optionally isolating the recombinant AAV composition produced from the host cell.

2. The method of embodiment 1, wherein the host cell is a mammalian cell engineered to express an AAV Rep gene and an AAV Cap gene.

3. The method of embodiment 2, wherein the exogenous DNA, the AAV Rep gene and the AAV Cap gene are stably integrated into the host cell genome and the AAV replication is initiated by infecting the host cell with an adenovirus or a herpes simplex 1 virus.

4. The method of embodiment 2, wherein the exogenous DNA, the AAV Rep gene, the AAV Cap gene, and adenoviral helper genes are introduced into the host cell by transient transfection, wherein the adenoviral helper genes comprise E4, E2a, and VA genes.

5. The method of any one of the preceding embodiments, wherein the ratio of the first AAV to the second AAV in the composition is about 0.5:1 to about 2:1, optionally about 1:1.

6. The method of any one of the preceding embodiments, wherein the bidirectional promoter is a minCBA promoter comprising a pair of chicken β-actin (CBA) promoters placed in opposite direction and separated by a CMV enhancer.

7. The method of embodiment 6, wherein the CMV enhancer comprises SEQ ID NO:8, or a nucleic acid sequence at least 85% identical thereto.

8. The method of embodiment 6, wherein the bidirectional promoter comprises SEQ ID NO:1 or 30, or a nucleic acid sequence at least 85% identical thereto.

9. The method of any one of the preceding embodiments, wherein the AAV ITRs are AAV2 ITRs.

10. The method of any one of embodiments 1-9, wherein the first transgene comprises a portion a full-length gene and the second transgene comprises the remainder of the full-length gene, wherein the full-length gene is 4.5 to 9 kb long, and said portion and said remainder each are no longer than 4.8 kb,
(a) wherein the first transgene comprises a splice donor at the 3′ end of its coding region and the second transgene comprises a splice acceptor at the 5′ end of its coding region, and wherein the splice donor and the splice acceptor promote generation of an RNA transcript of the full-length gene upon co-delivery of the first and second AAVs into a target cell; or
(b) wherein the 3′ coding region of the first transgene and the 5′ coding region of the second transgene overlap by 10 or more nucleotides, and wherein the overlap region promote generation of an RNA transcript of the full-length gene upon co-delivery of the first and second AAVs into a target cell.

11. The method of any one of embodiments 1-9, wherein the first and second transgenes each code for a different therapeutic protein.

12. The method of embodiment 11, wherein the first transgene encodes an anti-C1s antibody fragment, optionally an scFv or an scFab, and the second transgene encodes an anti-Bb antibody fragment, optionally an scFv or an scFab.

13. The method of embodiment 12, wherein the anti-C1s antibody comprises HCDR1-3 and LCDR1-3 comprising SEQ ID NOs:14-19, respectively;
V_{H} and V_{L} comprising SEQ ID NOs:20 and 21, respectively; or
SEQ ID NO:22 or 23.

14. he method of embodiment 12 or 13, wherein the anti-Bb antibody comprises HCDR1-3 and LCDR1-3 comprising SEQ ID NOs:4-9, respectively;
V_{H} and V_{L} comprising SEQ ID NOs:10 and 11, respectively; or
SEQ ID NO:12 or 13.

15. The method of embodiment 12, wherein the template AAV genome comprises SEQ ID NO:24, 25, 26, or 27.

16. The method of any one of the preceding embodiments, wherein the host cell is a mammalian cell, optionally a 293, HeLa, or A549 cell.

17. A recombinant AAV composition produced by the method of any one of embodiments 1-16.

18. A method of treating a disease in a human patient in need thereof, comprising delivering the recombinant AAV composition of embodiment 17 to the patient.

19. The recombinant AAV composition of embodiment 17 for use in treating a disease in a human patient in need thereof.

20. Use of the recombinant AAV composition of 17 for the manufacture of a medicament for treating a disease in a human patient thereof.

21. A recombinant AAV composition produced by the method of any one of embodiments 12-16.

22. A method of treating dry age-related macular degeneration (AMD) in a human patient in need thereof, comprising delivering the recombinant AAV composition of embodiment 21 to a diseased eye of the patient.

23. The recombinant AAV composition of embodiment 21 for use in treating dry AMD in a human patient in need thereof.

24. Use of the recombinant AAV composition of embodiment 21 for the manufacture of a medicament for treating dry AMD in a human patient in need thereof.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety, as if each individual reference were specifically and individually indicated to be incorporated by reference in its entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

According to the present disclosure, back-references in the dependent claims are meant as short-hand writing for a direct and unambiguous disclosure of each and every combination of claims that is indicated by the back-reference. Any compound disclosed herein can be used in any of the treatment method here, wherein the individual to be treated is as defined anywhere herein. Further, headers herein are created for ease of organization and are not intended to limit the scope of the claimed invention in any manner.

In order that this invention may be better understood, the following examples are set forth. These examples are for purposes of illustration only and are not to be construed as limiting the scope of the invention in any manner.

### EXAMPLES

### Example 1: Production of AAV Vector Genomes Containing a Bidirectional Promoter

The two wildtype (wt) ITRs that flank a classical rAAV genome are usually the only remaining sequences of viral origin and they play critical roles in AAV replication, packaging, and intracellular processing. Inclusion of shDNA-like palindromic sequences in the AAV vector genome have been shown to mediate strand switching during replication, whereby DNA polymerase can switch from using the leading strand to using either the lagging strand (inter-molecular strand switching) or the nascent strand (intra-molecular strand switching) as a template for replication (Xie et al., Mol Ther. (2017) 25(6):1363-74). FIG. 1A illustrates a scenario where the shDNA-like sequence directs intra-molecular strand switching, resulting in replication that U-turns back toward the wtITR without synthesizing the sequence beyond the hairpin structure (FIG. 1A, dotted line). As a result, truncated intra-molecular double-stranded genomes with loop regions centered at the shDNA sequence are generated for packaging (FIG. 1A). These structures are analogous to self-complementary AAV vector genomes (McCarty et al., Mol Ther. (2008) 16(10):1648-56). But in this example, the mutated ITR of self-complementary AAV vector genomes are replaced by the shDNA-like sequence or palindromic sequence of a bidirectional promoter. If replication overcomes the complementarity of the hairpin structure, the DNA polymerase continues to replicate the parental strand to completion, producing full-length AAV genomes (FIG. 1B).

In the present study, we studied the compositions of AAV products produced in a mammalian producer cell line engineered to produce an rAAV with a vector genome harboring a bi-directional promoter (minCBA) driving expression of two transgenes encoding anti-aC1s and anti-Bb single-chain Fab (scFab) (FIGS. 1D & 1E). We unexpectedly discovered that the AAV products contained primarily two populations of AAVs: a population of self-complementary anti-aC1s AAVs and a population of self-complementary anti-Bb AAVs (FIG. 6). During AAV production, upon replication, the vector genome forms two self-complementary, or “snap-back,” genomes, as shown in FIGS. 7B and 7C at a 1:1 ratio.

Without being bound by theory, we attribute this phenomenon to the existence of palindromic sequences in the bidirectional promoter.

A. rAAV Vector Genome

The bidirectional promoter was designed based on the ubiquitous minimal chicken j-actin (minCBA) promoter (FIG. 5B). This promoter supports the concurrent expression of individual antibody fragments to C1s and Bb. MinCBA contains a CBA promoter and an CMV enhancer but with an abbreviated intronic sequence. The bidirectional promoter contains a pair of CBA promoters placed in opposite directions and separated by an CMV enhancer (SEQ ID NO:7). The minCBA promoter harbors palindromic sequences (or shDNA-like sequences). Subsequent experiments (described below) show that those palindromic sequences behaved as surrogate inverted repeat elements promoting the formation of self-complementary AAV vector genomes during vector replication and production (FIGS. 2A-2C), leading to the production of two populations of AAV vectors: a self-complementary vector with the anti-aC1s vector sequence and a self-complementary vector with anti-Bb vector sequence only (FIG. 6).

In each scFab, glycine/serine-rich linkers (e.g., linkers with G₄S repeats) were inserted between the heavy and light chains of each single-chain αC1s and αBb Fab to facilitate proper folding of each antigen-binding domain formed by a pair of V_{H} and V_{L}. In the present studies, a linker with seven G₄S repeats was used to link the heavy and light chains of an scFab and a linker with three G₄S repeats was used to link the V_{H} and V_{L} of an scFv. The AAV vector genome studied here was the AAV2 #9 construct (SEQ ID NO:1). BHG-polyA.

Each transgene for the αC1s and αBb Fabs also contained bovine growth hormone (BGH) polyadenylation (polyA) signal sequences. The bicistronic expression cassette was cloned between AAV2 ITR sequences (see, e.g., FIG. 5B) for AAV delivery.

### B. Production of Recombinant AAVs

An AAV producer cell line (PCL) derived from HeLaS3 cells was produced as previously described (Martin et al., Hum Gene Ther Methods (2013) 24(4):253-69), using a triple-play plasmid encoding the AAV2 replicases and capsid proteins and an AAV vector genome (VG) as described above (AAV2 #9 construct). AAV production was initiated by infecting the PCL with wildtype adenovirus, as described in Martin et al., supra.

In addition, AAV production was performed using the triple transient transfection (TTx) production method as described by Nass et al., Mol Ther Methods Cin Dev. (2017) 9:33-46. The three plasmids used in the process included (1) a plasmid encoding the rAAV vector genome (FIG. 5B), (2) an AAV helper plasmid encoding the AAV replicases and capsid proteins, and (3) a pAd helper plasmid encoding the E4, E2a and VA genes (Nass et al., supra).

### C. Analysis of Vector DNA by Droplet Digital PCR

The AAV products generated from either production platform was purified using a two-step column purification method as described in Nass et al., supra. Purified AAV vector genomes were tittered using primers and probes against a) the anti-aC1s transgene sequence, b) the anti-Bb transgene sequence, or c) the BGH polyA sequence. Droplet digital PCR (ddPCR) data show that there were equivalent amounts of vector genomes harboring either the anti-aC1s vector genome or the anti-Bb vector genome. DdPCR analysis using primers and probes against the BGH polyA sequence, which would detect all vector genomes, indicates that there was twice the amount of vector with the BGH polyA sequence. The BGH polyA titer suggests that there were two populations of vector genomes with the BGH polyA sequence, consistent with there being two populations of vector genomes: one with the anti-aC1s-BGH vector genome sequence and the other with the anti-Bb-BGH polyA vector genome sequence.

The ratio of vectors expressing anti-Bb scFab to anti-aC1s scFab was approximately 1:1, suggesting that there was equivalent packaging of both vector genomes. DdPCR analysis of different rounds of rAAV production in the PCL platform and the TTx platform similarly reveal that the AAV products had equivalent amounts of packaged anti-aC1s vector genomes and anti-Bb vector genomes, and twice the amount of packaged vector genomes with BGH polyA (Table 1).

TABLE 1
DdPCR Analysis of rAAV Products

| | aC1s Primer/Probe | Bb Primer Probe | BGH |
|---|---|---|---|
| Vector Lot | (VG/ml) | (VG/ml) | (VG/ml) |
| ESB02-PCL | 1.43 × 10¹³ | 1.67 × 10¹³ | 2.6 × 10¹³ |
| CER01-PCL | 1.4 × 10¹³ | 1.8 × 10¹³ | ND |
| CER02-PCL | 1.5 × 10¹³ | 1.9 × 10¹³ | ND |
| X22122A - TTx | 2.08 × 10¹² | 2.18 × 10¹² | 4.60 × 10¹² |
| VP101622 - TTx | 5.79 × 10¹² | 6.10 × 10¹² | 1.30 × 10¹³ |

The above data show that the phenomenon of producing equivalent amounts of single-transgene rAAV vector genomes from a template with a bidirectional promoter was not related to the use of production method.

### D. Analysis of Vector DNA Under Alkaline and Non-Alkaline Conditions

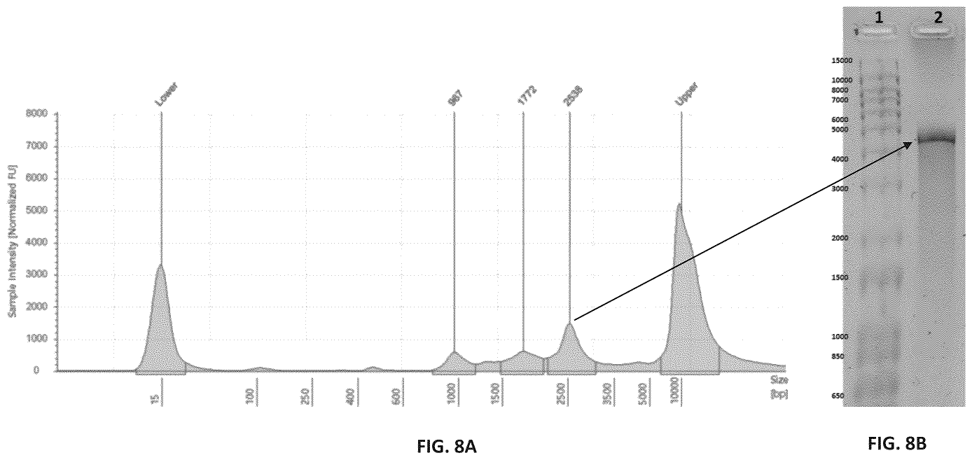

The DNA in the AAVs was extracted and analyzed under denaturing conditions in an alkaline gel or under non-alkaline conditions in a TapeStation®. The size of the extracted DNA as shown in the TapeStation® analysis was 2300 bp (FIG. 8A), which is half the expected size of the 4600 bp vector genome shown in FIG. 6, diagram A. In contrast, the same DNA analyzed under denaturing alkaline conditions reveals that the packaged DNA was about 4600 bp (FIG. 8B), suggesting that the extracted DNA was base-paired during TapeStation® analysis but single-stranded under alkaline conditions. The intra-strand base-pairing under non-alkaline conditions is characteristic of self-complementary AAV vectors due to the complementarity of strand base pairing. Base-pairing is disrupted under alkaline conditions, revealing the total size of the packaged AAV vector DNA to be 4600 bp, in agreement with the vector size of the input vector genome (FIGS. 1A and 1B).

### E. Analysis of AAV Vector Genome by PacBio® Sequencing

Four PCL and two TTx generated rAAV lots were prepared for long-read sequencing analysis by the PacBio® Sequel IIe system. In this experiment, a total of 5E11 rAAV VG per sample was subjected to DNase I treatment (RQ1 RNase-free DNase, Promega) to remove free-floating DNA in the sample. Following DNase I inactivation, rAAV DNA was extracted and purified using the PureLink™ Viral RNA/DNA Mini kit (Thermo Fisher Scientific) per manufacturer recommendations. Post DNA extraction, sizing of the purified rAAV DNA was confirmed by the 4200 TapeStation® system using the D5000 ScreenTape assay (Agilent). DNA concentration was determined by the Qubit™ Flex fluorometer (Thermo Fisher Scientific). Long-read DNA libraries were prepared using SMRTbell® Prep kit 3.0 (PacBio®) following manufacturer recommendations. Sequencing primer and polymerase were annealed to the long-read libraries using Sequel® II Binding kit 3.2. Completed libraries were sequenced using the Sequel® II Sequencing Plate 2.0 and Sequel® II internal control complex 3.2. A tool of 100 pM of pooled, complete library was run on a single SMRT® cell using the “AAV” sequencing mode in the SMRT® Link software v11.0.0.146107.

Images of long-read sequencing read length distribution from the seven rAAV samples were generated via the SMRT® Link software. The SMRT® Link software was also used to perform sequencing read alignments to the transgene reference and the generation of BAM files for visualization purposes.

To identify self-complementary DNA species and determine C1s and Bb transgene self-complementary ratios, a custom analysis pipeline was developed. The pipeline utilizes BLASTN, which compares a nucleotide query sequence (i.e., long-read sequences determined by the Sequel IIe system) against a custom database containing all the elements of the rAAV transgene cassette. These sequences include the 5′ and 3′ ITRs, as well as the two transgenes of interest (anti-C1s and anti-Bb scFab), the BGH polyA tail, and the promoter and enhancer. Briefly, a long-read sequencing output file (.fasta format) for each sample containing all of the sequencing reads specific to that sample was passed against the database containing the transgene sequences using the BLASTN program. Each individual read was then annotated, in the 5′ to 3′ orientation, such that a pattern of each element generated for each read was included in a .fasta file. The main script of the pipeline parsed the BLASTN output and groups reads that had similar detected patterns. Each group was then processed by sorting the start and stop position of each element to ensure full-length sequential coverage of each element. The output was compiled into an Excel file, which could be sorted to determine reads with multiple copies of anti-C1s or anti-Bb scFab transgene, as well as the pattern of the other defined elements in individual sequencing reads. Relative percentages of any identified element pattern were determined by dividing the number of reads specific to a certain pattern by the total number of sequencing reads and multiplying by 100.

Sequencing read alignments were visualized using the SMRT® Link generated BAM files and Integrative Genomics Viewer (IGV) genomics data visualization tool (data not shown).

The data show that the majority of DNA species, regardless of rAAV manufacturing platform or lot, was detected at about 2300 bp, which is approximately half of the expected VG size of 4600 bp. These data support the presence of self-complementary vector genome transgene configurations (i.e., intramolecular base-pairing under non-alkaline conditions).

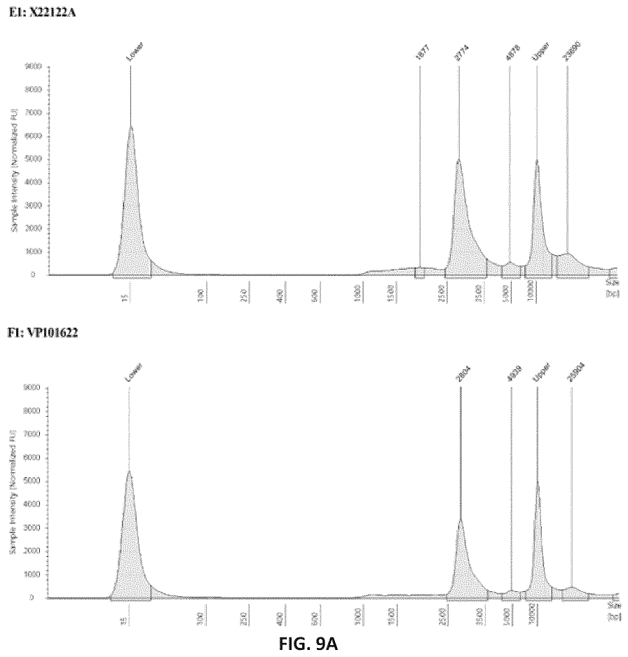

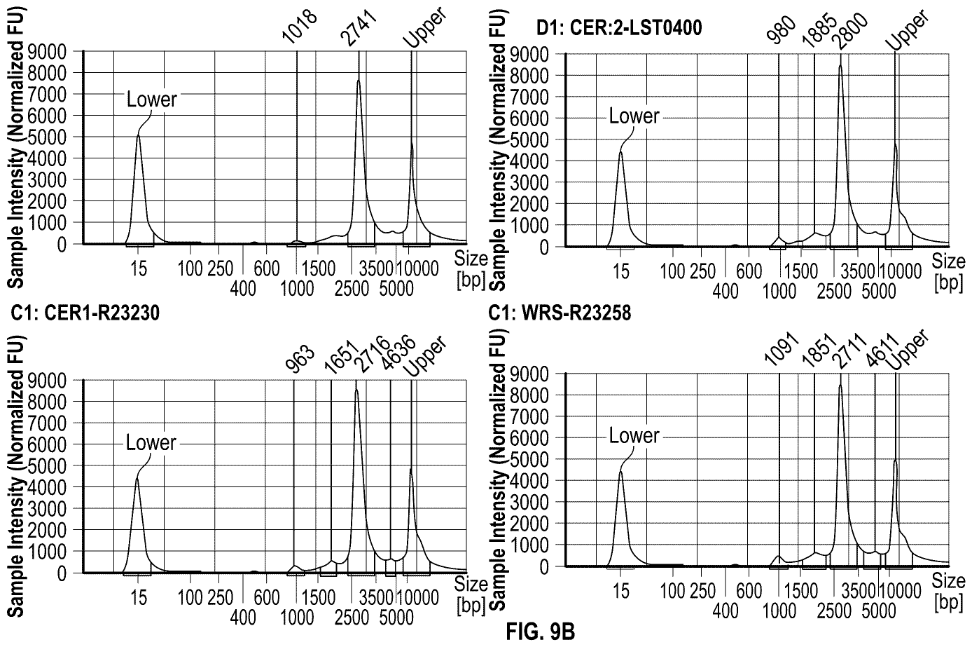

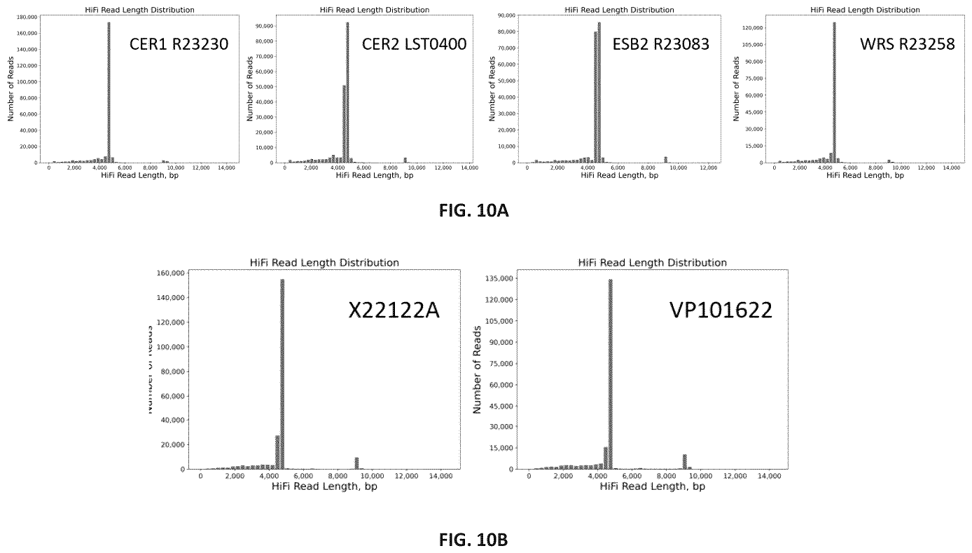

As shown in FIGS. 10A and 10B, the majority of reads were detected at the expected transgene size of 4600 bp, regardless of manufacturing platform or material lot. This is in conflict with the results shown in FIGS. 9A and 9B, where the majority species of the extracted rAAV DNA was detected at half of the expected transgene size.

Collectively, the data support the presence of a self-complementary transgene configuration, specifically when considering that the long-read sequencing was performed in “AAV” mode. During long-read sequencing DNA library preparation, the input DNA typically has two blunt ends for sequencing adapter ligation. This allows for sense and antisense strand determinations during SMRT® sequencing on the Sequel® IIe platform. When running in “Amplicon” mode, any library that contains a single sequencing adapter is flagged by the software as an incomplete library and the read generated for this library is removed during quality assessments of the sequencing run.

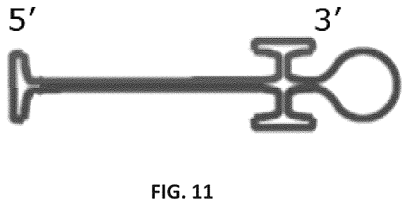

A self-complementary rAAV VG only contains a single open end for adapter ligation (i.e., at the 5′ and 3′ ITR junction), whereas the other end contains the pinch point for self-complementary snap-back onto the designed reverse complement sequence (i.e., non-functional delta-ITR region) (FIG. 11). The “AAV” sequencing run mode was implemented by PacBio® as an alternative to the “Amplicon” mode, allowing for the retention of reads containing a single adapter and the analysis of self-complementary rAAV vectors. The fact that the predominant half-sized rAAV DNA species, as detected by the 4200 TapeStation® under non-alkaline conditions, resolved to read lengths at the expected transgene size for all vector lots analyzed by long-read sequencing provides strong evidence that the packaged transgene is in a self-complementary configuration.

The self-complementary transgene configurations can be clearly identified by IGV alignment from sequencing reads of PCL-derived vector when using the expected bi-directional transgene sequence. For both the C1s-C1s and Bb-Bb configurations, the sequence misalignments are due to the unexpected inclusion of the reverse complement of the gene, thus verifying two unique self-complementary species, each containing two copies of a single gene (data not shown).

To further investigate the self-complementary transgene configuration, the BLASTN pipeline was run on the long-read sequencing data to determine transgene element pattern and relative quantitation. The outcome of these analyses confirmed that the expected, bi-directional transgene configuration was not the predominant species; instead, two self-complementary species, including the gene C1s-C1s or Bb-Bb orientations were prevalent, with the intact promoter-enhancer-promoter elements present in the bi-directional transgene sequencing acting as the folding point of the self-complementary transgenes.

The data in Table 2 and Table 3 show the relative percentages from the BLASTN analyses, normalized to total reads per samples, of the Bb-Bb and C1s-C1s self-complementary configurations, as well as the ratio between the two species, for the PCL-derived and TTx-generated vector lots, respectively.

TABLE 2
Sequel ® IIe Long-Read Sequencing Data from PCL Vector Lots

| | | | Bb-Bb to |
|---|---|---|---|
| PCL Vector | Transgene Configuration (% of Reads) | | C1s-C1s |
| Lot | Bb-Bb | C1s-C1s | Ratio |
| R23258 (Initial) | 39.25 | 28.74 | 1.4 |
| R23258 (Repeat) | 40.50 | 30.45 | 1.3 |
| R23230 | 40.73 | 30.81 | 1.3 |
| LST0400 | 40.48 | 30.14 | 1.4 |
| R23083 | 44.88 | 32.71 | 1.4 |

TABLE 3
Sequel ® IIe Long-Read Sequencing Data from TTx Vector Lots

| TTx Vector | Transgene Configuration (% of Reads) | | Bb-Bb to C1s- |
|---|---|---|---|
| Lot | Bb-Bb | C1s-C1s | C1s Ratio |
| X22122A | 39.15 | 30.22 | 1.3 |
| VP101622 | 35.30 | 29.85 | 1.2 |

As shown in Tables 2 and 3, the relative percentages and ratios of Bb-Bb and C1s-C1s self-complementary configurations for all vectors tested, regardless of manufacturing platform and lot, are similar. Table 2 also includes results from two unique sample testing occasions for PCL lot R23258, which yielded high-similar results.

### Example 2: The Bidirectional MinCBA Promoter with shDNA-Like Sequences Produces Functional Double-Stranded rAAV Vector Genomes

Wildtype, female C57Bl/6J mice aged 8-12 weeks were administered formulation buffer, or a TTX-VP101622 or PCL-ESB02 rAAV preparation at 5×10⁶, 5×10⁷, 5×10⁸, or 5×10⁹ VG per eye through bilateral, intravitreal injections (n=8-10 mice per study group). Dosing was based on the anti-aC1s transgene specific titer assay. Retinal tissue was collected from all mice on Day 29 and nucleic acid was extracted. Retina tissue samples were homogenized in lysis buffer. Following homogenization, retinal lysates were centrifuged to remove debris. Genomic DNA and total RNA were then extracted from the retina lysates using the Qiagen AllPrep® DNA/RNA/Protein Mini Purification Kit (Qiagen, 80004) and nucleic acid extracted VG levels were measured by quantitative (qPCR) using 500 ng DNA input and a primer probe assay specific for the anti-aC1s transgene sequence in the vector. Anti-aC1s and anti-Bb transcript levels were measured by reverse transcription-quantitative PCT (RT-qPCR) using complementary DNA (cDNA) equivalent to 30 ng RNA input and primer probe assays specific for either the anti-aC1s or anti-Bb transgene sequences.

In TTX-VP101622-treated animals, the median±MAD VG levels were 1024.7 483.1, 1.00×10⁴±3325.5, 6.87×10⁴±3.64×10⁴, and 1.22×10⁵±3.69×10⁴ VG/500 ng DNA in the 5×10⁶, 5×10⁷, 5×10⁸, and 5×10⁹ VG/eye dose groups, respectively (dose-response P<0.0001).

In PCL-ESB02-treated animals, the median±MAD VG levels were 570.3±203.5, 9706.6±6085.2, 3.70×10⁴±1.70×10⁴, and 9.99×10⁴±3.56×10⁴ VG/500 ng DNA in the 5×10⁶, 5×10⁷, 5×10⁸, and 5×10⁹ VG/eye dose groups, respectively (dose-response P<0.0001).

Statistical analyses of these data show that vector genome levels for each vector genome, aC1s and Bb in eyes transduced with TTX-VP101622 and PCL-ESB02 are comparable.

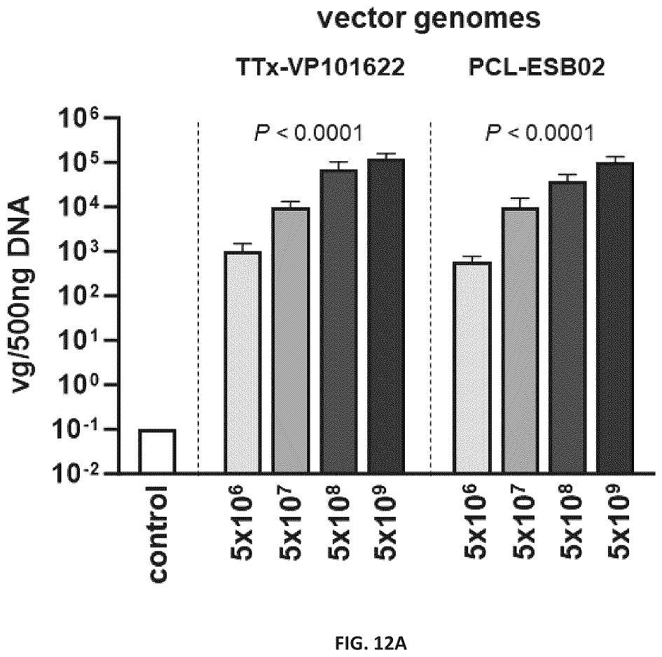

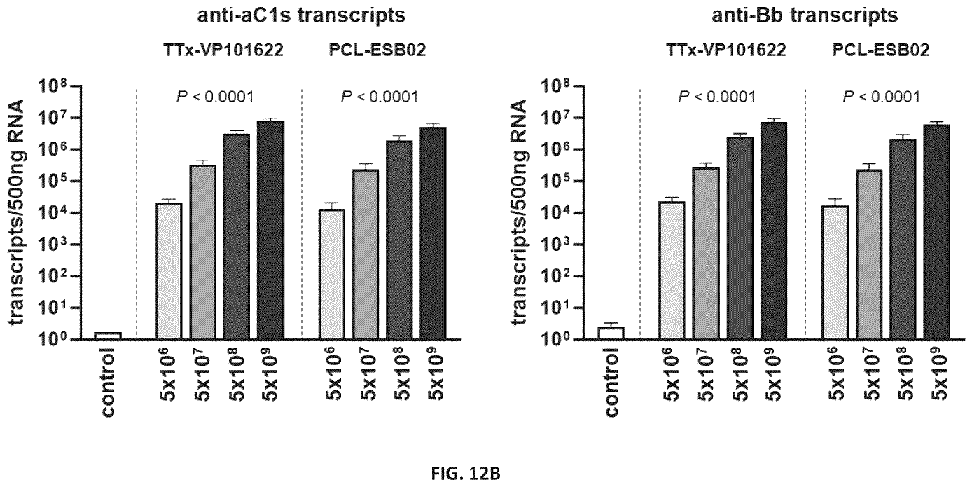

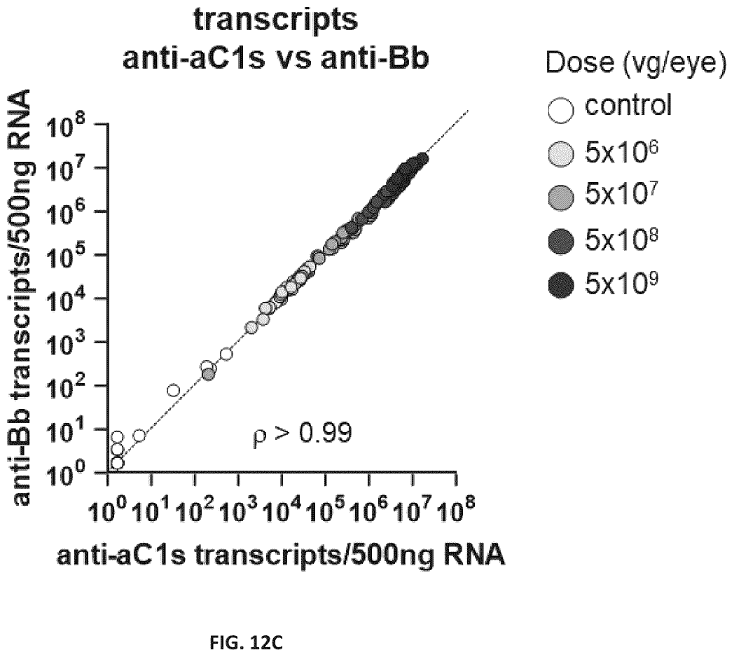

Levels of vector-derived anti-aC1s and anti-Bb transcripts in the mouse retina were quantified using vector-specific anti-aC1s and anti-Bb TaqMan® assays in RT-qPCR analyses. For both TTX-VP101622 and PCL-ESB02, vector transduction resulted in a dose-dependent increase in anti-aC1s and anti-Bb transcript copies (FIGS. 12A and 12B). Furthermore, TTX-VP101622 and PCL-ESB02 each produced equivalent levels of anti-aC1s and anti-Bb transcripts, which were highly correlated (spearman r>0.99) (FIG. 12C).

This study demonstrates that TTx-VP101622 and PCL-ESB02 had comparable in vivo potency following intravitreal injection into the wildtype mouse retina. Retinal transduction by either TTx-VP101622 or PCL-ESB02 resulted in comparable levels of anti-aC1s transcripts and anti Bb transcripts. Moreover, there was a dose-dependent increase in both vector genomes and transcript copies.

### Example 3: Formation of Double-Stranded Snap-Back rAAV Vector Genomes

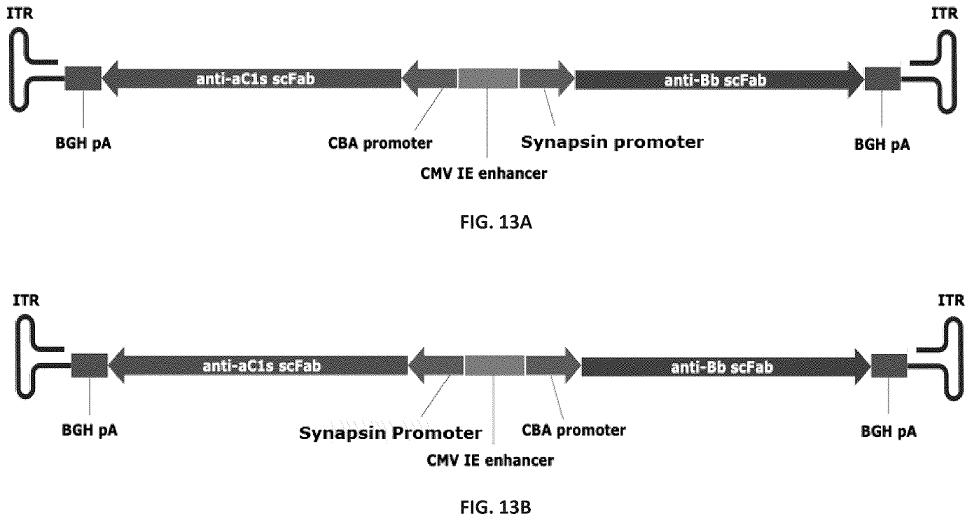

To prevent the formation of snap-back genomes, the vector was redesigned to include a synapsin promoter driving expression of the second transgene (FIGS. 13A and 13B), while retaining the central CMV enhancer sequence.

A. Packaging and Purification of rAAV Vector Plasmid

The ITR vector plasmid was packaged into an AAV.SAN024 capsid using transient triple transfection, following the protocol described by Nass et al., supra. Briefly, HEK293 cells were transfected using polyethyleneimine with a 1:1:1 ratio of three plasmids: the AAV vector, the AAV rep/cap plasmid, and the adenoviral helper plasmid. The rep/cap plasmid contained rep sequences from AAV2 and capsid sequences from AAV.SAN024. The adenoviral helper plasmid used was pHelper (Stratagene/Agilent Technologies, Santa Clara, CA).

AAV vectors were purified using affinity column chromatography (AVB Sepharose High-Performance medium; GE Healthcare), followed by enrichment for full capsids via cesium chloride (CsCl) gradient centrifugation, as described by Nass et al., Supra. The proportion of empty versus genome-containing capsids was assessed by AUC, following the method of Burnham et al., Hum Gene Ther Methods (2015) 26(6):228-42.

Vector titers were quantified using ddPCR with primer probes targeting one of the following sequences: the bovine growth hormone polyadenylation site, the aC1s transgene, and the Factor Bb vector genome (Table 4).

TABLE 4
ddPCR Results of AAV.SAN024
CBA-hSyn-aC1s-Bb Lot VP031125

| | BGH | aC1s | Bb1 |
|---|---|---|---|
| Sample | (VGs/mL) | (VGs/mL) | (VGs/mL) |
| Post-CsCl VP031125MB | 6.92E12 | 4.14E12 | 4.11E12 |
| Post-CsCl VP031125BB | 2.49E12 | 1.61E12 | 1.54E12 |

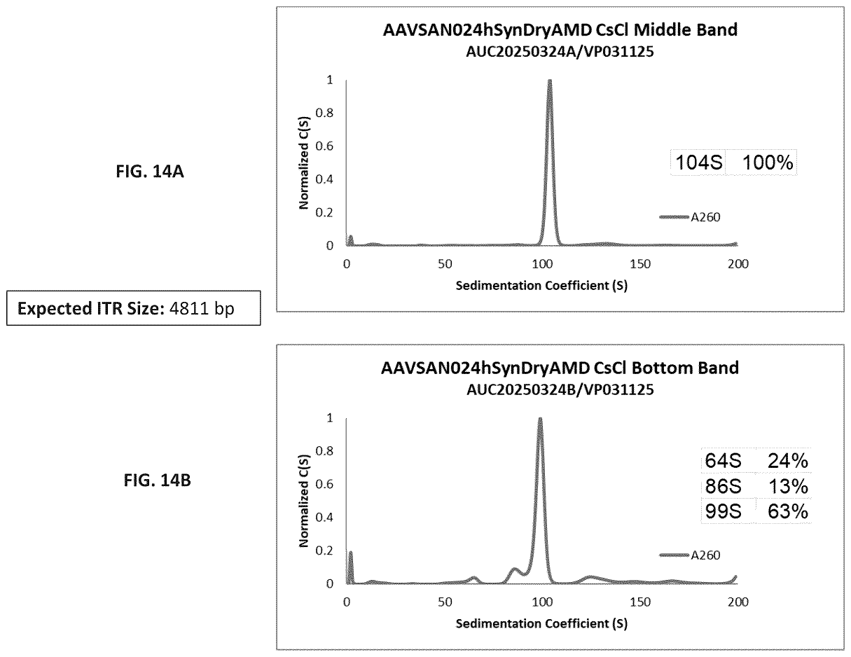

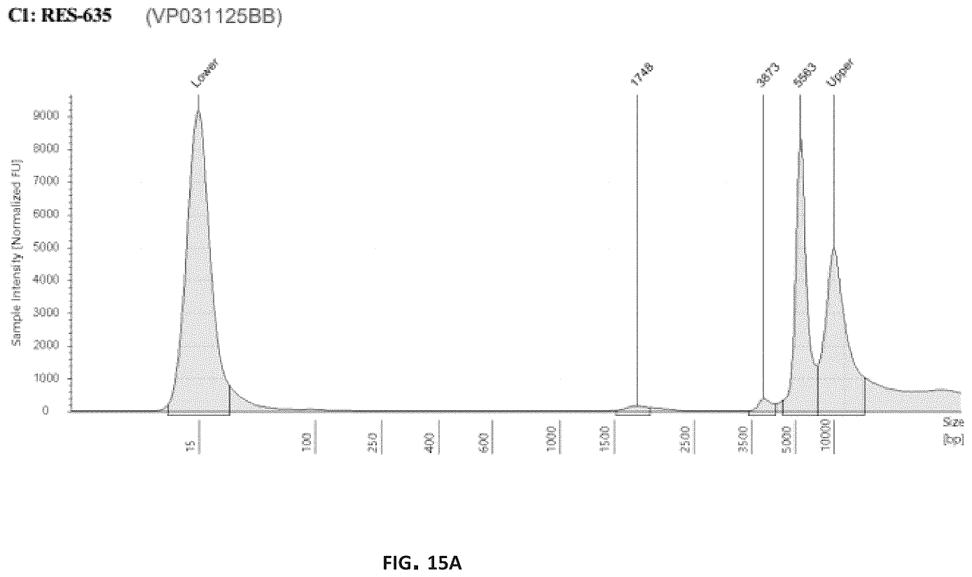

Following CsCl purification, two distinct vector populations were recovered: VP031125 MB and VP031125BB. Both were analyzed by AUC and shown to be enriched for genome containing particles, as illustrated in FIGS. 14A and 14B.

B. Analysis of rAAV Vector Genome by PacBio® Sequencing

Both VP031125 MB and VP031125BB vectors were subjected to PacBio® long read sequencing as described before. Briefly, at least 2.7 vg of rAAV vector, per sample, was subjected to DNase I treatment (DNase I RNase-free kit, New England Biolabs) to remove free-floating DNA in the sample. Following DNase I inactivation per the recommendations of the manufacturer, rAAV DNA was extracted and purified using the PureLink™ Viral RNA/DNA Mini kit (Thermo Fisher Scientific) per manufacturer recommendations. Post DNA extraction, sizing of the purified rAAV DNA was confirmed by the 4200 TapeStation® system using the D5000 ScreenTape™ assay (Agilent). DNA concentration was determined by the Qubit™ Flex fluorometer (Thermo Fisher Scientific). Long-read DNA libraries were prepared using SMRTbell® Prep kit 3.0 (PacBio®) following manufacturer recommendations. Sequencing primer and polymerase were annealed to the long-read libraries using Sequel II Binding kit 3.1. Completed libraries were sequenced using the Sequel II Sequencing Plate 2.0 and Sequel II internal control complex 3.1. A pool of completed libraries, at 125 pM, was run on a single SMRT® cell using the “AAV” sequencing mode in the SMRT® Link software.

Images of long-read sequencing read length distribution from the rAAV samples were generated via SMRT® Link software v 11.0.0.146107. SMRT® Link software was also used for the generation of BAM files to support subsequent analyses.

To identify the pattern, and mean length, of different rAAV genomic species, a custom analysis pipeline was developed and run on the Sanofi Magellan™ analysis platform. The pipeline utilizes BLASTN which compares a nucleotide query sequence (i.e., long-read sequences determined by the Sequel IIe system) against a custom database containing all the elements of the rAAV transgene cassette. These sequences include the 5′ and 3′ITRs, the genes of interest, BGH polyA tail, the promoter, and the enhancer. Briefly, a long-read sequencing output file (BAM format) for each sample containing all of the sequencing reads specific to that sample was passed against the database containing the sequences using the BLASTN program. Each individual read was then annotated, in the 5′ to 3′ orientation, such that a pattern of each element was generated for each read included in the BAM file. The main script of the pipeline parses the BLASTN output and groups reads that have similar detected patterns. Each group is then processed by sorting the start and stop position of each element to ensure full-length sequential coverage of each element. The output is compiled into an Excel file which can be sorted to determine the number of sequencing reads containing unique patterns, as well as the mean sequencing length for each unique pattern. Relative percentages of any identified element pattern can be determined by dividing the number of reads specific to a certain pattern by the total number of sequencing reads and multiplying by 100.

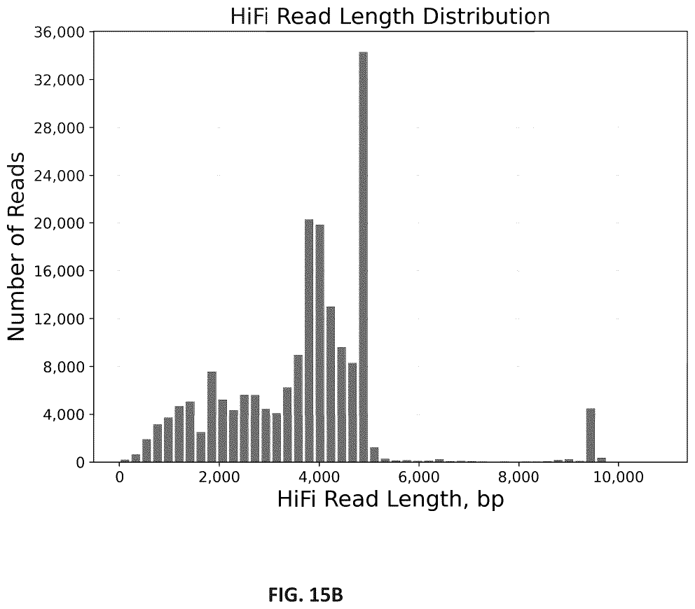

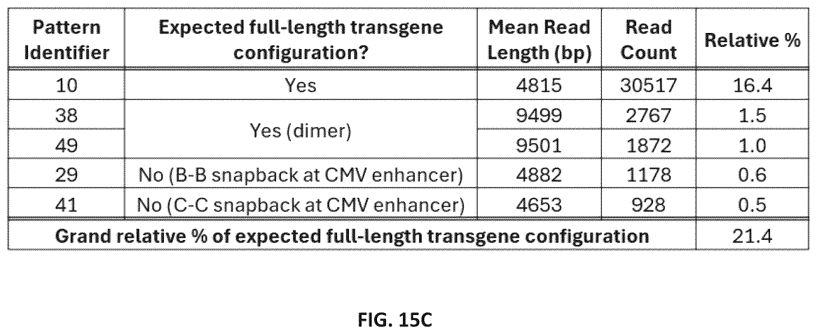

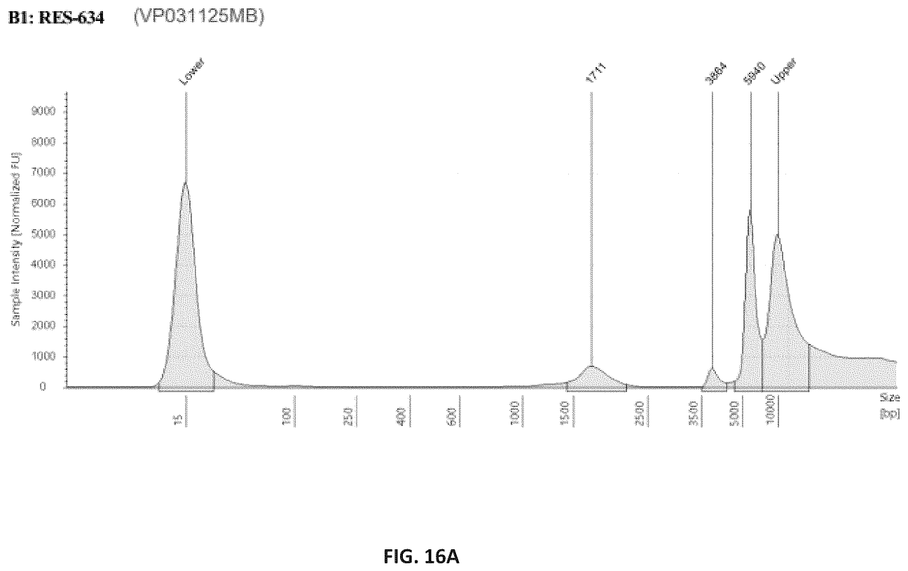

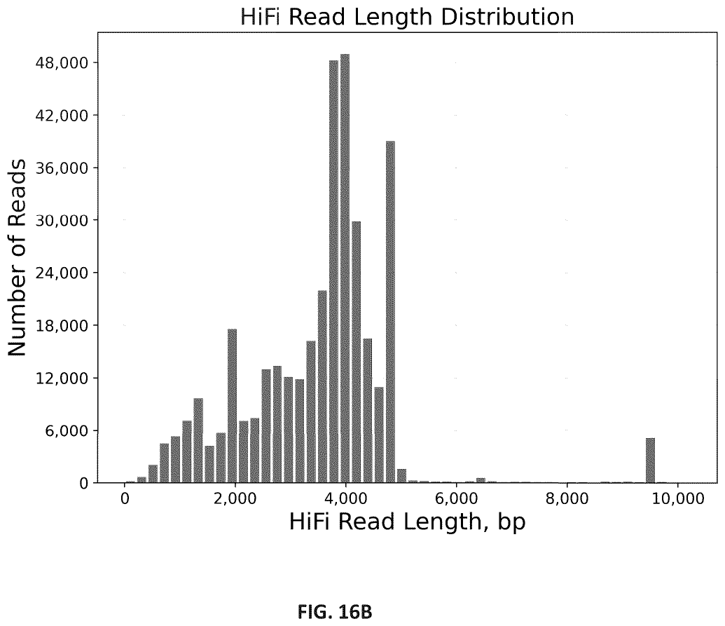

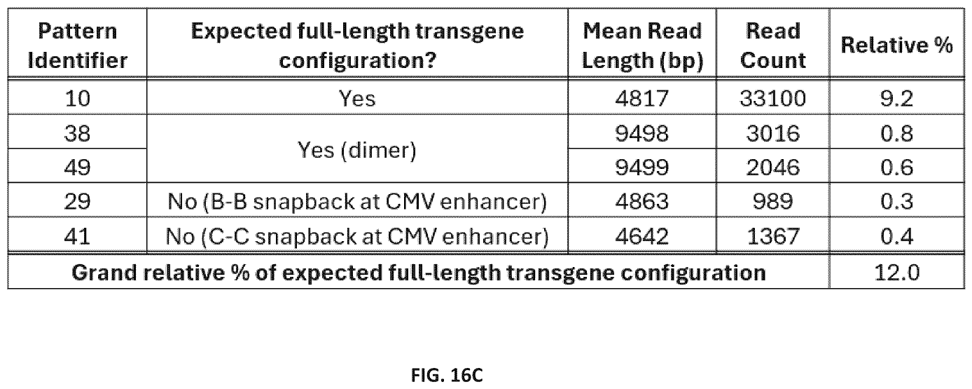

A summary of the long-read sequencing results for AAV.SAN024 CBA-hSyn-aC1s-Bb lots VP031125BB and VP031125 MB are shown in FIGS. 15A-15C and 16A-16C, respectively. The images of FIGS. 15A and 16A show the size profile of extracted rAAV DNA, under non-denatured conditions, as determined by the 4200 TapeStation® system (Agilent). FIGS. 15B and 16B include sequencing read length (bp) distributions. FIGS. 15C and 16C include the transgene configuration patterns detected by BLASTN analysis specific to the expected full-length rAAV transgene (patterns 10, 38, and 49), and product specific snap-back vectors forming around the CMV enhancer (patterns 29 and 41). It is important to note that dimer patterns 38 and 49 contain the expected forward and reverse full-length transgene sequences with an ITR sequence in the middle. It is hypothesized that this is an artifact of long-read sequencing artifact (i.e., end-repair and ligation of ITR “D”-regions), and the species are separated into two full-length transgene reads for total relative quantitation reporting.

As shown in FIGS. 15A and 16A, the extracted rAAV DNA profiles are similar between lots VP031125 MB and VP031125BB, with a predominate peak detected approximately at the expected transgene length (5940 and 5563 bp, respectively). There is good correlation between the DNA sizing profiles (FIGS. 15A and 16A) and sequencing read length distribution (FIGS. 15B and 16B), where a larger population of shorter/truncated species are being detected in lot VP031125 MB (FIGS. 16A-16C) compared to VP031125BB (FIGS. 15A-15C) by both analyses. The BLASTN analysis for lot VP031125 MB identified 13538 unique patterns in 360687 processed sequencing reads, and 8874 unique patterns in 186074 processed sequencing reads for lot VP031125BB.

Collectively, the patterns specific to expected, full-length bicistronic transgene configurations are most abundant at a combined total of 33.4% (sum of “grand relative % of expected full-length transgene configuration” in FIGS. 15C and 16C). This value is >20-fold and ˜6-fold higher compared to lots produced using the bidirectional CBA promoter configuration by producer cell line and triple-transfection, respectively. Further, product specific snap-back patterns around the CMV enhancer were <1% for both Bb and C1s species (FIGS. 15C and 16C), which is ˜30-fold lower compared to rAAV vectors generated using the bidirectional CBA promoter configuration.

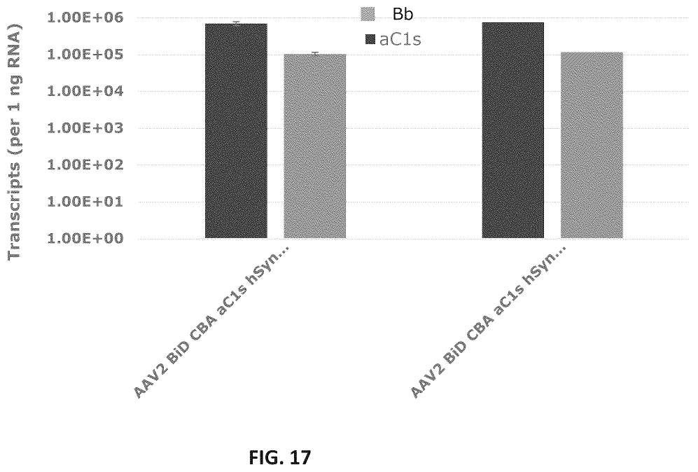

In the vector genome configuration shown in FIGS. 13A and 13B—where one transgene is driven by the CBA promoter and the other by the synapsin promoter, both enhanced by a shared CMV sequence—the predominant sequencing reads of the packaged vector genomes, correspond to the expected full-length genome (˜4.8 kb). FIG. 17 shows aC1s and Bb scFab transcript driven by the CBA vs. human synapsin (hSyn) promoters in AAV infected 293 cells. AAV2-BiD-CBA-aC1s-hSyn-Bb scFab Lot #VP031125 MB at multiplicity of infection (MOI) of 1E5 VG/cell. AAV2-BiD-CBA-aC1s-hSyn-Bb scFab Lot #VP031125BB at MOI=1E5 VG/cell. Ad5ts149 MOI=1. The total infection time was 48 hours.

FIGS. 15A-15C and 16A-16C (Pattern_10) indicate that approximately 33% of the packaged genomes for combined vectors VP031125BB and VP031125 MB are full-length, consistent with the schematic in FIGS. 13A and 13B. Notably, this configuration shows minimal evidence (<1%) of packaging self-complementary, snap-back genomes as illustrated in FIGS. 7B and 7C. In contrast, the bidirectional CBA promoter configuration (FIG. 7A), previously described, results in the packaging of two distinct, self-complementary genomes at an about 1:1 ratio (FIGS. 7B and 7C).

### Example 4: Generation of a Population of Predominantly Double-Stranded Self-Complementary AAV (scAAV) Vector Genomes

A. scAAV Vector Genomes

Self-complementary adeno-associated viral (scAAV) vectors represent a significant advancement in gene therapy delivery systems. Unlike conventional AAVs that require second-strand DNA synthesis after cellular entry, scAAVs contain a modified genome that forms a double-stranded DNA structure through self-annealing, due to a mutation in one of the ITRs.

During replication of the self-complementary vector genome terminal resolution at the mutated ITR does not occur. This promotes replication through the delta ITR, and a second strand is generated using the newly synthesized strand as a template. The result is a self-complementary or double-stranded vector genome, limited to the AAV packaging capacity of 4.6 kb. However, a limitation of this process is that replication through the delta ITR is error-prone, often resulting in terminal resolution and generation of single-stranded monomeric vector genomes of 2.3 Kb (Nass et al., supra).

To generate a population of self-complementary AAV vectors that are predominantly double-stranded (>70%) with minimal monomeric species, we employed the use of a minCBA bidirectional promoter instead of a mutated ITR with a delta terminal resolution site (trs).

Self-complementary adeno-associated viral (scAAV) vectors offer a significant improvement in gene therapy delivery by bypassing the need for second-strand DNA synthesis. This unique design bypasses the rate-limiting step of second-strand synthesis, resulting in significantly faster and more efficient transgene expression. scAAVs demonstrate enhanced transduction efficiency in various tissues, particularly in non-dividing cells like neurons, retinal cells, and hepatocytes, making them invaluable for treating conditions affecting these tissues. Their rapid onset of expression and improved transduction efficiency make scAAVs particularly valuable for applications requiring immediate therapeutic effects or targeting tissues where conventional AAV transduction is suboptimal. Unlike conventional AAVs, scAAVs utilize a mutated inverted terminal repeat (ITR) that enables the genome to self-anneal into a double-stranded structure during replication. This process avoids terminal resolution at the mutated ITR, allowing replication through the delta ITR and formation of a complementary strand using the newly synthesized DNA as a template. However, due to imperfect fidelity at the delta ITR, some single-stranded monomeric genomes (˜2.3 kb) may still be produced, as noted by Nass et al., supra. The total genome size remains constrained by the AAV packaging limit of ˜4.6 kb.

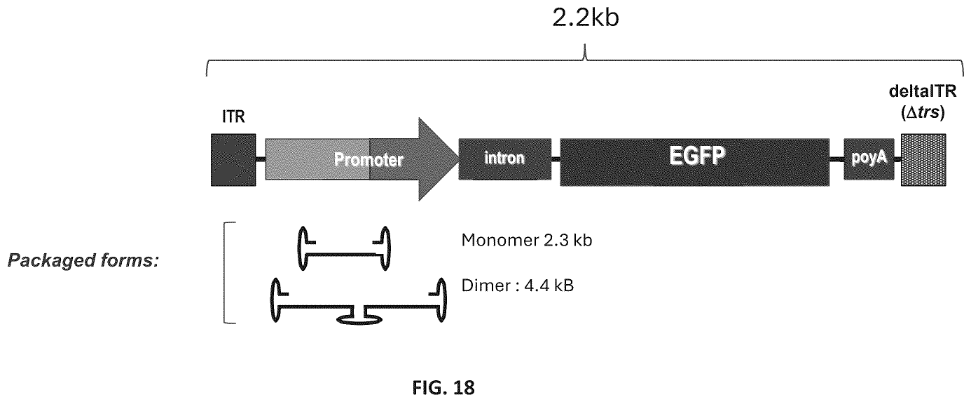

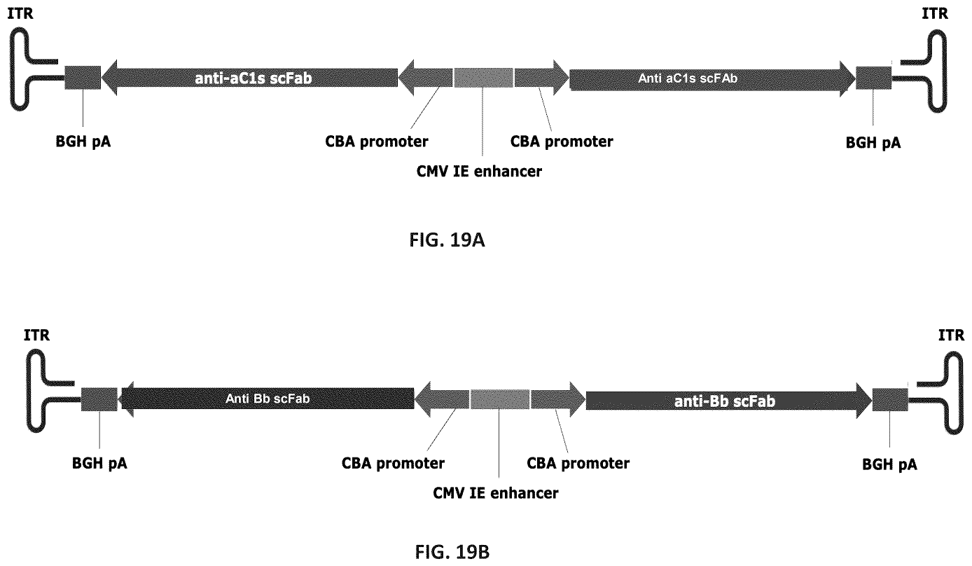

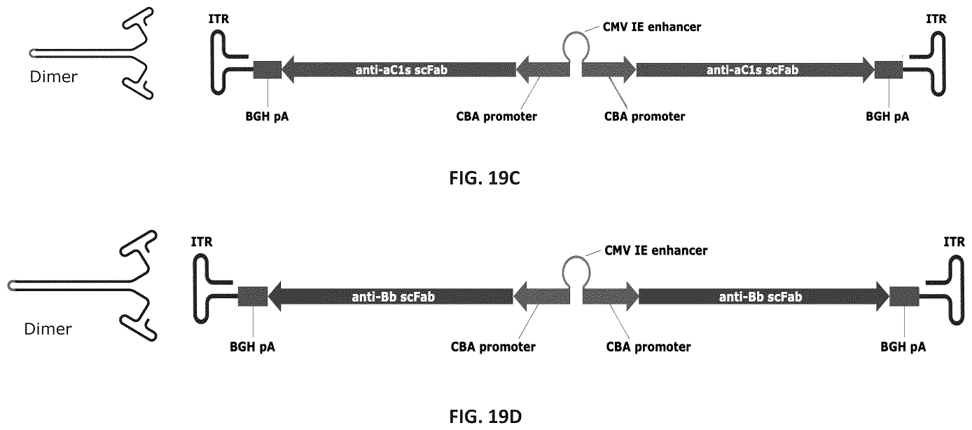

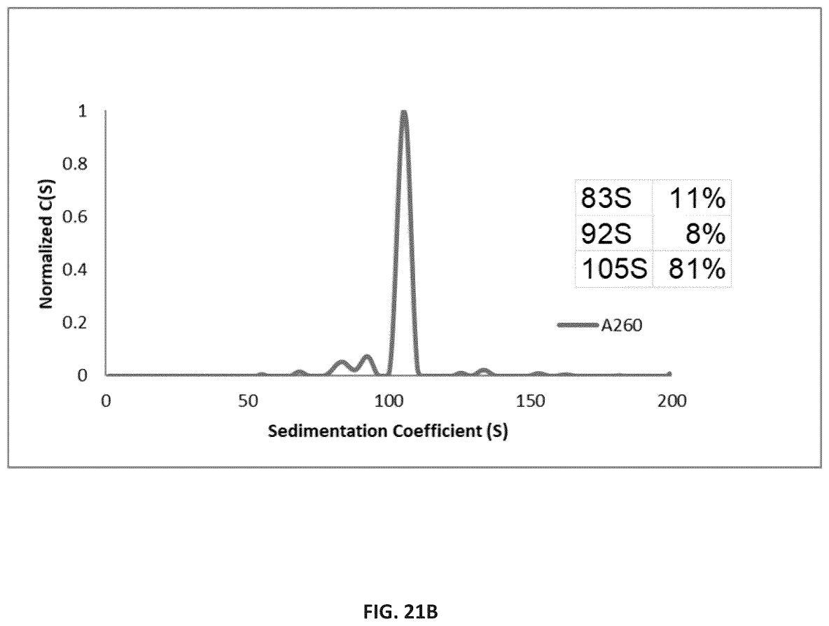

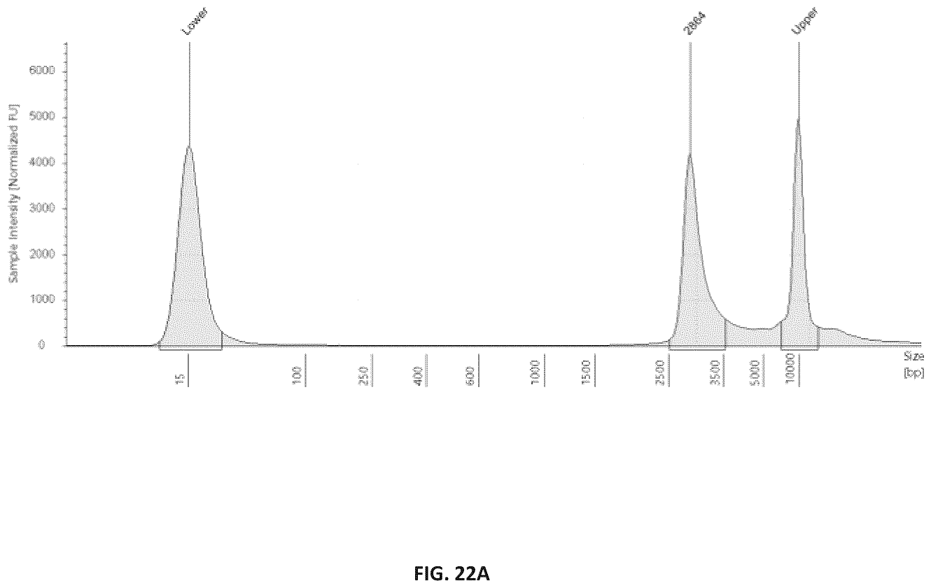

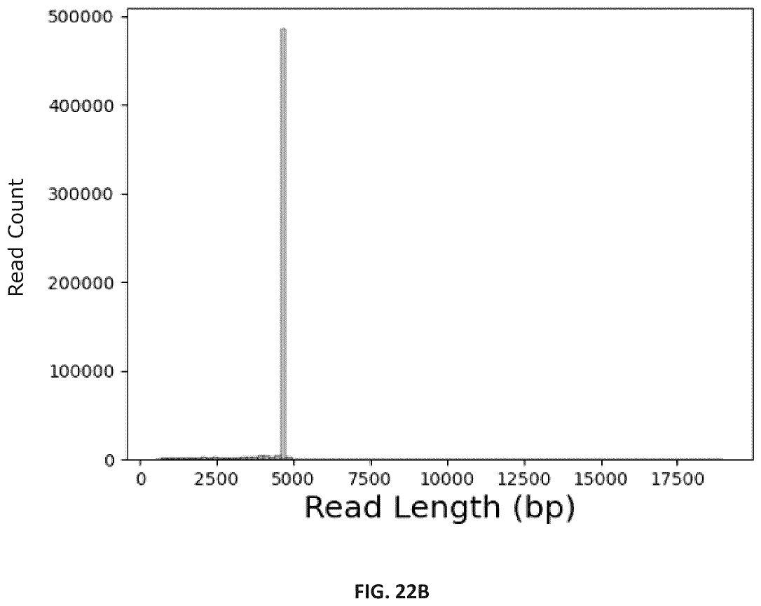

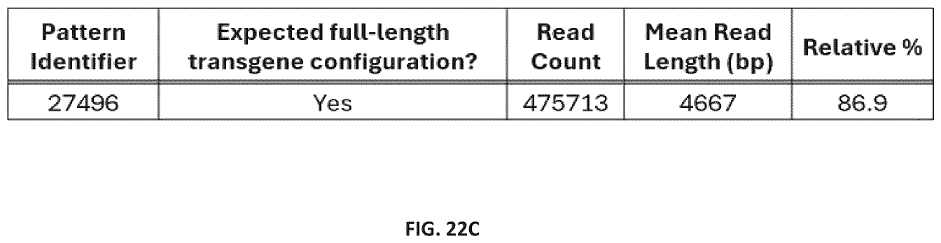

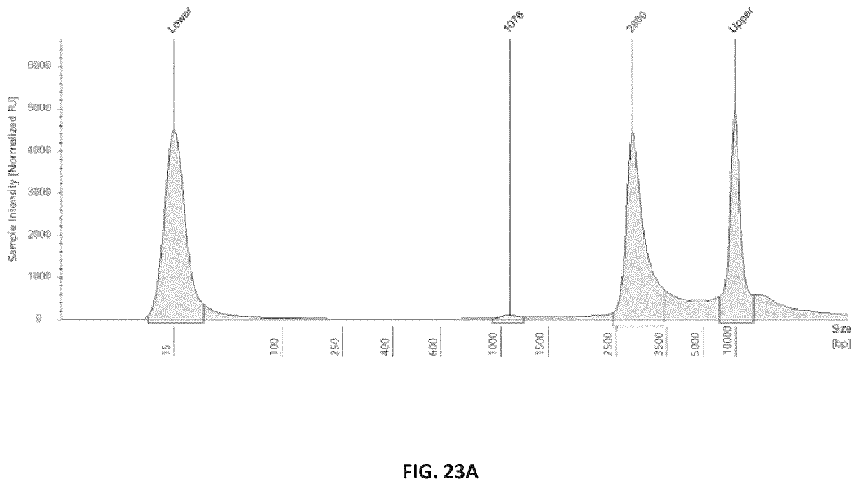

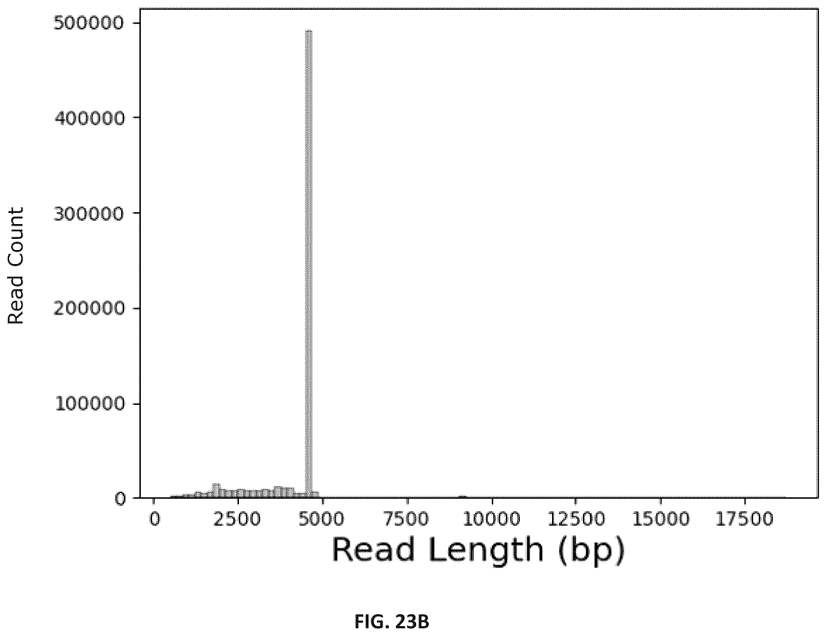

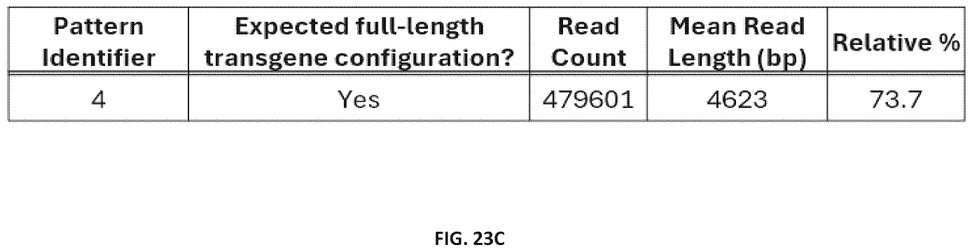

To enhance the proportion of double-stranded scAAV genomes (>70%) and minimize monomeric species, we used the minCBA bidirectional promoter (FIGS. 19A and 19B) instead of a mutated ITR with a delta trs (FIG. 18). The inverted repeat sequences within the minCBA promoter facilitate snap-back replication, resulting in predominantly double-stranded vector genomes (FIGS. 19C and 19D). This outcome is supported by analytical ultracentrifugation (FIGS. 21A, and 21B) and PacBio® sequencing data (FIGS. 22A-22C and 23A-23C).

Accordingly, key findings of the present studies were: (i) utilizes minCBA bidirectional promoter (FIGS. 19A and 19B); (ii) replaces mutated ITR with delta trs (FIG. 18); (iii) vector genome replication results in a snap-back, self-complementary structure; and (iv) inverted repeat sequences in the CBA promoter facilitate double-stranded AAV vector formation (FIGS. 19C and 19D). The evidence of efficacy included: minimal monomeric species observed, and results were verified by AUC (FIGS. 21A, and 21B) and PacBio® sequencing (FIGS. 22A-22C and 23A-23C).

B. Fractional Content of Packaged Dimeric and Monomeric rAAV VG

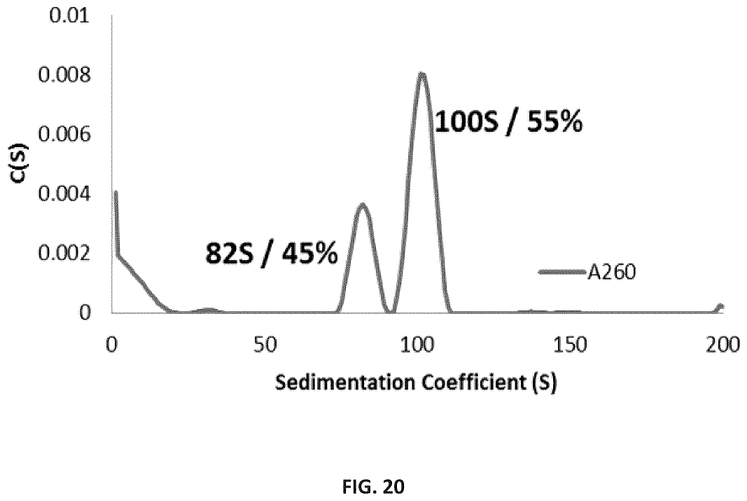

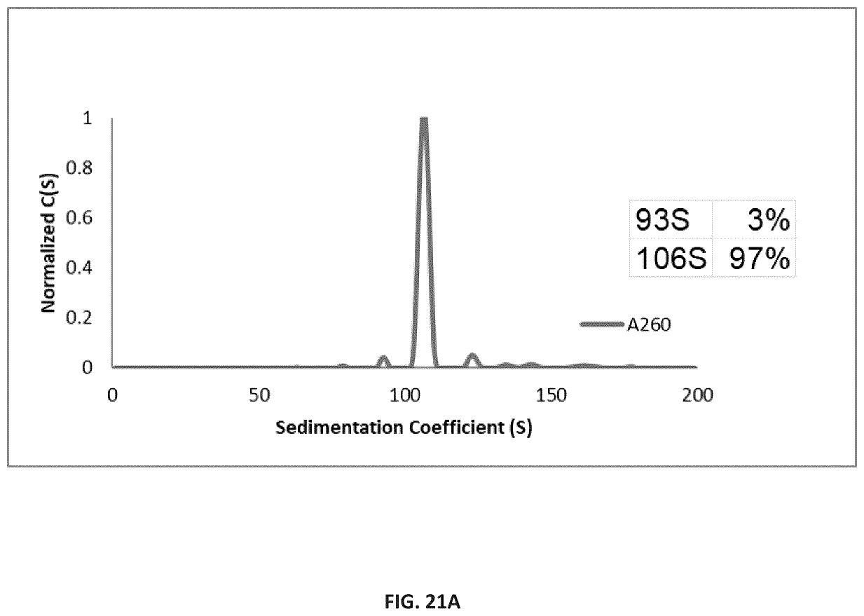

ITR plasmids as shown in FIGS. 19A and 19B were generated. The nucleic acid sequences of SEQ ID NOs.: 31 and 32 were used to generate rAAV vectors in the context of the AAV.SAN024 capsid. rAAV vectors were generated using the triple transfection protocol as described by (Nass et al., supra). The purified rAAV was subjected to analytical centrifugation as described before (Burnham et al, supra) to assess the fractional content of packaged dimeric and monomeric vector genomes. FIGS. 21A and 21B reveal that for both AAV.SAN024.anti-aC1s-scFab (FIG. 21A) and AAV.SAN024.anti.Bb.scFab (FIG. 21B), there is a major peak sedimenting with an S value consistent with packaging a dimeric vector genome that is 4.6 kb (105S), with little evidence of packaging the 2.3 kb monomeric species expected to sediment at 85S (Burnham et al., supra). In contrast, FIG. 20 shows that for a typical self-complementary vector, scAAV2EGFP-generated using the mutated delta ITR with the trs (terminal resolution site) mutated-only 55% vector genome species are dimeric, sedimenting at 100S (consistent with packaging a species of 4.4 kb). A significant amount is monomeric species, sedimenting at 82S (consistent with packaging a vector species that is 2.2 kb).

### C. Analysis of AAV Vector Genome by PacBio® Sequencing

Two AAV lots, X25230 and X25223 (transgene configurations shown in FIGS. 19C and 19D, respectively), were subjected to PacBio® long-read sequencing as previously described. Briefly, at least 2.1E12 vg of rAAV vector, per sample, was subjected to DNase I treatment (DNase I RNase-free kit, New England Biolabs) to remove free-floating DNA in the sample. Following DNase I inactivation per the recommendations of the manufacturer, rAAV DNA was extracted and purified using the PureLink™ Viral RNA/DNA Mini kit (Thermo Fisher Scientific) per manufacturer recommendations. Post DNA extraction, sizing of the purified rAAV DNA was confirmed by the 4200 TapeStation® system using the D5000 ScreenTape assay (Agilent). DNA concentration was determined by the Qubit™ Flex fluorometer (Thermo Fisher Scientific). Long-read DNA libraries were prepared using SMRTbell® Prep kit 3.0 (PacBio®) following manufacturer recommendations. Sequencing primer and polymerase were annealed to the long-read libraries using Sequel II Binding kit 3.1. Completed libraries were sequenced using the Sequel II Sequencing Plate 2.0 and Sequel II internal control complex 3.1. A pool of completed libraries, at 100 pM, was run on a single SMRT® cell using the “AAV” sequencing mode in the SMRT® Link software.

Images of long-read sequencing read length distribution from the rAAV samples were generated via SMRT® Link software v 11.0.0.146107. SMRT® Link software was also used for the generation of BAM files to support subsequent analyses.

To identify the pattern, and mean length, of different rAAV genomic species, a custom analysis pipeline was developed and run on the Sanofi Magellan™ analysis platform.

A summary of the long-read sequencing results for AAV lots X25230 and X25223 are shown in FIGS. 22A-22C and FIG. 23A-23C, respectively. The images of FIG. 22A and FIG. 23A show the size profile of extracted rAAV DNA, under non-denatured conditions, as determined by the 4200 TapeStation® system (Agilent). The images of FIG. 22B and FIG. 23B include sequencing read length (bp) distributions. FIGS. 22C and 23C include the transgene configuration patterns detected by BLASTN analysis specific to the expected full-length rAAV transgene, including detected number, mean base pair length, and the relative percentage.

As shown in FIGS. 22A and 23A, the extracted rAAV DNA profiles are similar between lots X25230 and X25223, with predominate peaks detected at approximately half the size of the expected transgene lengths (2864 and 2800 bp, respectively). The read length size distribution images (FIGS. 21A and 21B) show the majority of reads to be at the expected vector length of ˜4.7 kb. BLASTN pattern analyses for lot X25230 yielded 2917 unique patterns in 547550 processed sequencing reads, and 6164 unique patterns in 650926 processed sequencing reads. The expected transgene configuration was the most abundant pattern for both lots, with the aC1s snap-back around the bidirectional CBA promoter composing 86.9% of the sequencing reads of lot X25230 (FIG. 22C) and 73.7% of the sequencing reads for lot X25223 (Bb snap-back around the bidirectional CBA promoter (FIG. 23C).

Collectively, the long-read sequencing results from AAV lots X25230 and X25223 support a snap-back configuration of the aC1s and Bb transgenes, respectively, around the bidirectional CBA promoter element. Although the 4200 TapeStation® showed extracted DNA profiles of half-sized vectors (FIGS. 22A and 23A), this is the expected sizing for non-denatured analyses due to the self-complementary nature of the sequence on either side of the CMV enhancer. The vector genome spontaneously folds on itself after capsid release and is characterized as half-sized during electrophoresis analysis. The length of the full vector transgene is captured by the read lengths of long-read sequencing, with both lots showing a majority of reads at the expected ˜4.7 kb size (FIGS. 22B and 23B). This provides further evidence of the self-complementary transgene configuration, as long-read sequencing can provide uninterrupted sequence confirmation of a DNA library through the recognition of a sequencing adapter. During library preparation, a scAAV vector will have a single sequencing adapter ligated at the open end of the species (i.e., junction between the 5′ and 3′ ITR). During the sequencing of the library, the entire sequence between the ITRs will be read, resulting in the entire 4.7 kb genome size being resolved. Subsequent BLASTN analyses of the long-read sequencing data confirmed that the majority of the 4.7 kb reads were in the expected scAAV configuration for lots X25230 (aC1s scAAV at 86.9% of all reads, FIG. 22C) and X25223 (Bb scAAV at 73.7%, FIG. 23C). The PacBio® long-read sequencing data supports the claim that the bidirectional CBA promoter with CMV enhancer element and can be used as a reliable alternative to mutated ITRs for efficient scAAV generation and packaging.

SEQUENCES
SEQ ID NO: 1 - Bidirectional promoter and CMV enhancer (CBA
promoter (reverse) bolded and underlined; CMV enhancer boldfaced and
italicized; CBA promoter boxed)

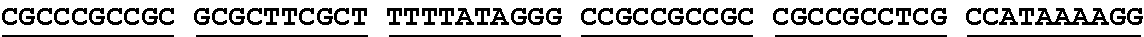

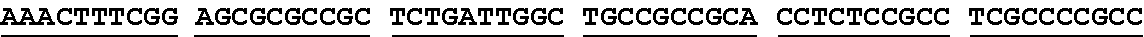

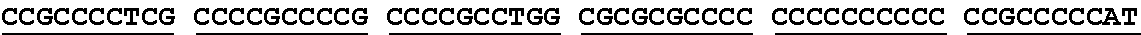

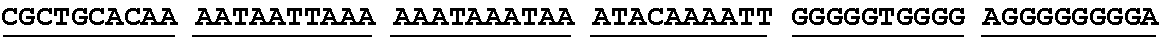

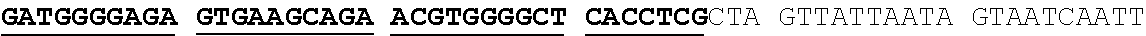

ACGGGGTCAT TAGTTCATAG CCCATATATG GAGTTCCGCG TTACATAACT TACGGTAAAT
GGCCCGCCTG GCTGACCGCC CAACGACCCC CGCCCATTGA CGTCAATAAT GACGTATGTT
CCCATAGTAA CGCCAATAGG GACTTTCCAT TGACGTCAAT GGGTGGAGTA TTTACGGTAA
ACTGCCCACT TGGCAGTACA TCAAGTGTAT CATATGCCAA GTACGCCCCC TATTGACGTC
AATGACGGTA AATGGCCCGC CTGGCATTAT GCCCAGTACA TGACCTTATG GGACTTTCCT

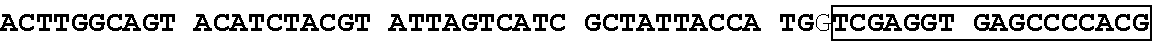

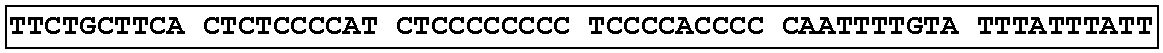

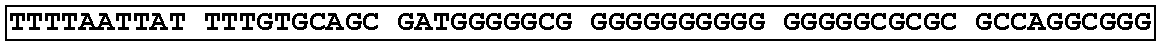

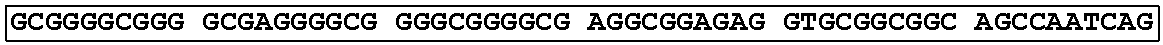

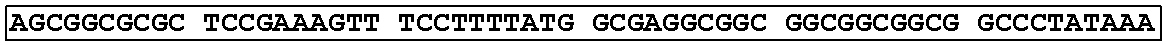

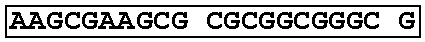

SEQ ID NO: 2 - C3A promoter
TCGAGGTGAG CCCCACGTTC TGCTTCACTC TCCCCATCTC CCCCCCCTCC CCACCCCCAA
TTTTGTATTT ATTTATTTTT TAATTATTTT GTGCAGCGAT GGGGGCGGGG GGGGGGGGGG
GGCGCGCGCC AGGCGGGGCG GGGCGGGGCG AGGGGCGGGG CGGGGCGAGG CGGAGAGGTG
CGGCGGCAGC CAATCAGAGC GGCGCGCTCC GAAAGTTTCC TTTTATGGCG AGGCGGCGGC
GGCGGCGGCC CTATAAAAAG CGAAGCGCGC GGCGGGCG
SEQ ID NO: 3 - CMV Enhancer
CGTTACATAA CTTACGGTAA ATGGCCCGCC TGGCTGACCG CCCAACGACC CCCGCCCATT
GACGTCAATA ATGACGTATG TTCCCATAGT AACGCCAATA GGGACTTTCC ATTGACGTCA
ATGGGTGGAG TATTTACGGT AAACTGCCCA CTTGGCAGTA CATCAAGTGT ATCATATGCC
AAGTACGCCC CCTATTGACG TCAATGACGG TAAATGGCCC GCCTGGCATT ATGCCCAGTA
CATGACCTTA TGGGACTTTC CTACTTGGCA GTACATCTAC GTATTAGTCA TCGCTATTAC
CATG
SEQ ID NO: 4 - HCFR1 of anti-Cls antibody
DDYIH
SEQ ID NO: 5 - HCDR2 of anti-Cls antibody
RIDPADGHTK YAPKFQV
SEQ ID NO: 6 - HCDR3 of anti-Cls antibody
YGYGREVEDY
SEQ ID NO: 7 - LCDR1 of anti-Cls antibody
KASQSVDYDG DSYMN
SEQ ID NO: 8 - LCDR2 of anti-Cls antibody
DASNLES
SEQ ID NO: 9 - LCDR3 of anti-Cls antibody
QQSNEDPWT
SEQ ID NO:10 - V_{H} of anti-Cls antibody (Kabat CDRs underlined)
QVQLVQSGAE VKKPGASVKL SCTASGENIK DDYIHWVKQA PGQGLEWIGR IDPADGHTKY
APKFQVKVTI TADTSTSTAY LELSSLRSED TAVYYCARYG YGREVEDYWG QGTTVTVSS
SEQ ID NO: 11 - VL of anti-Cls antibody (Kabat CDRs underlined)
DIVLTQSPDS LAVSLGERAT ISCKASQSVD YDGDSYMNWY QQKPGQPPKI LIYDASNLES
GIPARFSGSG SGTDFTLTIS SLEPEDFAIY YCQQSNEDPW TFGGGTKVEI K
SEQ ID NO: 12 - αCls scFab (V_{L} underlined; G/S linker linking the
light chain and the heavy chain italicized; V_{H} boldfaced; CDRs
boxed)
DIVLTQSPDS LAVSLGERAT ISCKASQSVD YDGDSYMNWY QQKPGQPPKI LIYDASNLES
GIPARFSGSG SGTDFTLTIS SLEPEDFAIY YCQQSNEDPW TFGGGTKVEI KRTVAAPSVF
IFPPSDEQLK SGTASVVCLL NNFYPREAKV QWKVDNALQS GNSQESVTEQ DSKDSTYSLS
STLTLSKADY EKHKVYACEV THQGLSSPVT KSFNRGECGG GGSGGGGSGG GGSGGGGSGG
GGSGGGGSGG GGSQVQLVQS GAEVKKPGAS VKLSCTASGF NIKDDYIHWV KQAPCQGLEW

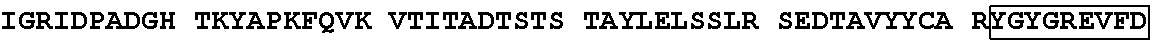

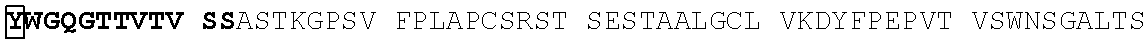

GVHTFPAVLQ SSGLYSLSSV VTVPSSSLGT KTYTCNVDHK PSNTKVDKRV
SEQ ID NO: 13 - αCls scFab-CM arm (construct #22, FIG. 2B)(signal
sequence boldfaced; charge mutations boxed and italicized, numbering
excluding signal peptide: Q42E and Q292K)
MEAPAQLLFL LLLWLPDTTG DIVLTQSPDS LAVSLGERAT ISCKASQSVD YDGDSYMNWY

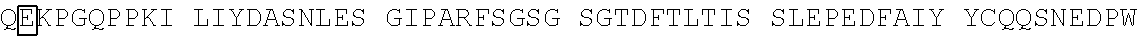

TFGGGTKVEI KRTVAAPSVF IFPPSDEQLK SGTASVVCLL NNFYPREAKV QWKVDNALQS
GNSQESVTEQ DSKDSTYSLS STLTLSKADY EKHKVYACEV THQGLSSPVT KSFNRGECGG
GGSGGGGSGG GGSGGGGSGG GGSGGGGSGG GGSQVQLVQS GAEVKKPGAS VKLSCTASGF

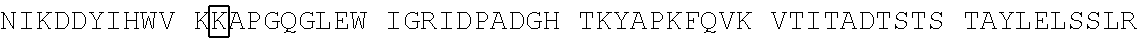

SEDTAVYYCA RYGYGREVFD YWGQGTTVTV SSASTKGPSV FPLAPCSRST SESTAALGCL
VKDYFPEPVT VSWNSGALTS GVHTFPAVLQ SSGLYSLSSV VTVPSSSLGT KTYTCNVDHK
PSNTKVDKRV*
SEQ ID NO: 14 - HCDR1 of anti-Bb antibody
NYAMS
SEQ ID NO: 15 - HCDR2 of anti-Bb antibody
TISNRGSYTY YPDSVKG
SEQ ID NO: 16 - HCDR3 of anti-Bb antibody
ERPMDY
SEQ ID NO: 17 - LCDR1 of anti-Bb antibody
KASQDVGTAV A
SEQ ID NO: 18 - LCDR2 of anti-Bb antibody
WASTRHT
SEQ ID NO: 19 - LCDR3 of anti-Bb antibody
HQHSSNPLT
SEQ ID NO: 20 - V_{H} of anti-Bb antibody (Kabat CDRs boxed)

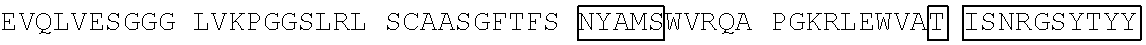

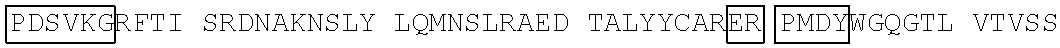

SEQ ID NO: 21 - V_{L} of anti-Bb antibody (Kabat CDRs boxed)

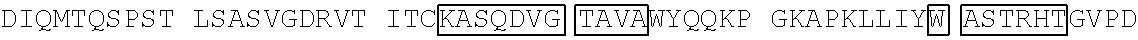

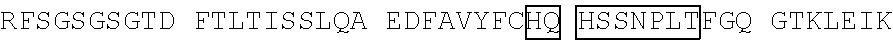

SEQ ID NO: 22 - αBb scFab (V_{L} underlined; G/S linker linking the
light chain and the heavy chain italicized; VH boldfaced)
DIQMTQSPST LSASVGDRVT ITCKASQDVG TAVAWYQQKP GKAPKLLIYW ASTRHTGVPD
RFSGSGSGTD FTLTISSLQA EDFAVYFCHQ HSSNPLTFGQ GTKLEIKRTV AAPSVFIFPP
SDEQLKSGTA SVVCLLNNFY PREAKVOWKV DNALQSGNSQ ESVTEQDSKD STYSLSSTLT
LSKADYEKHK VYACEVTHQG LSSPVTKSEN RGECGGGGSG GGGSGGGGSG GGGSGGGGSG
GGGSGGGGSE VQLVESGGGL VKPGGSLRLS CAASGFTFSN YAMSWVRQAP GKRLEWVATI
SNRGSYTYYP DSVKGRFTIS RDNAKNSLYL QMNSLRAEDT ALYYCARERP MDYWGQGTLV
TVSSASTKGP SVEPLAPCSR STSESTAALG CLVKDYFPEP VTVSWNSGAL TSGVHTEPAV
LQSSGLYSLS SVVTVPSSSL GTKTYTCNVD HKPSNTKVDK RV
SEQ ID NO: 23 - αBb scFab-CM arm (construct #22, FIG. 2B) (signal
sequence boldfaced; charge mutations boxed and italicized, numbering
excluding signal peptide: Q38K and Q288E, and S114A, N137K, and
T434E)

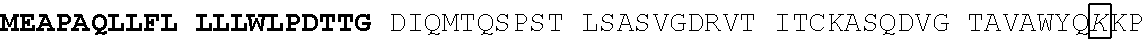

GKAPKLLIYW ASTRHTGVPD RFSGSGSGTD FTLTISSLQA EDFAVYFCHQ HSSNPLTFGQ

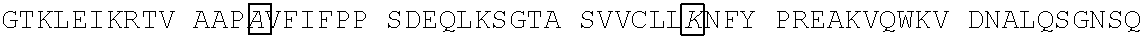

ESVTEQDSKD STYSLSSTLT LSKADYEKHK VYACEVTHQG LSSPVTKSEN RGECGGGGSG
GGGSGGGGSG GGGSGGGGSG GGGSGGGGSE VQLVESGGGL VKPGGSLRLS CAASGETESN

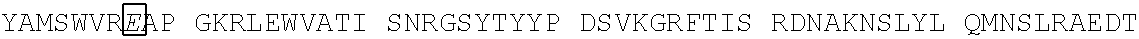

ALYYCARERP MDYWGQGTLV TVSSASTKGP SVEPLAPCSR STSESTAALG CLVKDYFPEP

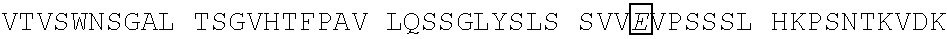

RV*
SEQ ID NO: 24 - Nucleotide sequence of AAV2#9 [αCls scFab -
bidirectional promoter - xBb scFab] (FIGs. 4A) (5′ ITR boldfaced;
bGH polyA signal underlined; reverse complement of aCls scFab coding
sequence italicized; IgG kappa signal coding sequence italicized and
underlined; Kozak sequence boxed; CBA promoter (reverse) bolded and
underlined; CBA promoter boxed and underlined; CMV enhancer
boldfaced and italicized; xBb scFab boldfaced, italicized, and
underlined; and 3′ ITR boxed and italicized)
TTGGCCACTC CCTCTCTGCG CGCTCGCTCG CTCACTGAGG CCGCCCGGGC AAAGCCCGGG
CGTCGGGCGA CCTTTGGTCG CCCGGCCTCA GTGAGCGAGC GAGCGCGCAG AGAGGGAGTG
GCCAACTCCA TCACTAGGGG TTCCTTACAA TTCTAGTTCC CCAGCATGCC TGCTATTGTC
TTCCCAATCC TCCCCCTTGC TGTCCTGCCC CACCCCACCC CCCAGAATAG AATGACACCT
ACTCAGACAA TGCGATGCAA TTTCCTCATT TTATTAGGAA AGGACAGTGG GAGTGGCACC
TTCCAGGGTC AAGGAAGGCA CGGGGGAGGG GCAAACAACA GATGGCTGGC AACTAGAAGG
CACAGGTTTA AACCCTGCAG GGAGCTCTCA CACCCGCTTA TCCACCTTGG TGTTGCTGGG
CTTGTGGTCC ACGTTGCAGG TGTAGGTCTT TGTGCCCAGG CTAGAGCTAG GCACTGTCAC
GACAGAGGAC AGAGAGTACA GGCCGCTGCT CTGCAGCACG GCGGGGAAGG TGTGCACCCC
GCTTGTCAGG GCTCCGCTGT TCCAGGACAC GGTCACAGGC TCAGGGAAAT AATCCTTGAC
CAGGCAGCCC AGAGCAGCCG TGCTCTCTGA GGTACTTCTG CTACAAGGAG CCAGTGGGAA
CACGCTAGGG CCCTTTGTGC TGGCGGACGA CACGGTCACT GTTGTGCCCT GTCCCCAGTA
GTCGAACACT TCTCTGCCGT AGCCGTATCT GGCGCAGTAG TACACAGCGG TGTCCTCGGA
TCTAAGGCTG CTCAGTTCCA GATAAGCTGT AGAGGTGCTG GTATCGGCGG TGATGGTGAC
TTTCACCTGG AACTTAGGGG CGTACTTTGT GTGGCCGTCG GCAGGGTCGA TTCTGCCGAT
CCACTCCAGT CCCTGGCCGG GGGCCTGCTT CACCCAGTGG ATGTAATCGT CCTTGATATT
GAAGCCGCTG GCGGTGCAGC TCAGCTTAAC ACTAGCGCCA GGCTTTTTCA CCTCGGCTCC
GCTCTGCACC AGCTGCACCT GGGATCCGCC GCCGCCGCTG CCGCCTCCGC CGCTGCCGCC
TCCGCCGCTT CCGCCTCCCC CAGAGCCGCC GCCACCGCTG CCTCCTCCGC CGGAGCCGCC
GCCGCCGCAC TCGCCCCGGT TGAAGCTTTT GGTCACAGGA GAGGACAGGC CCTGATGTGT
CACTTCACAG GCGTACACCT TGTGCTTCTC GTAGTCGGCC TTGCTCAAGG TCAGGGTGCT
GGACAGGCTG TATGTTGAGT CCTTGCTGTC CTGCTCGGTC ACGCTCTCTT GGCTGTTGCC
GCTTTGCAGG GCGTTGTCAA CTTTCCATTG GACCTTTGCC TCTCTGGGGT AGAAGTTATT
CAGCAGGCAC ACCACAGAGG CGGTTCCGCT CTTCAGCTGC TCGTCGCTTG GAGGGAAGAT
AAAGACAGAA GGGGCGGCCA CGGTGCGCTT GATTTCCACC TTGGTGCCGC CTCCAAAGGT
CCAGGGGTCC TCGTTGCTCT GCTGGCAGTA GTAGATGGCA AAATCCTCGG GTTCCAGAGA
AGAAATTGTC AGGGTGAAAT CAGTGCCAGA GCCGCTGCCG CTGAATCTGG CGGGGATGCC
GCTTTCCAGA TTGCTGGCGT CGTAGATCAG GATTTTTGGA GGCTGGCCGG GTTTCTGCTG
GTACCAGTTC ATGTAGCTGT CGCCGTCATA GTCCACGCTC TGAGAGGCTT TACAGCTGAT
TGTGGCCCGT TCGCCGAGGC TCACGGCCAG GCTATCAGGG CTCTGCGTCA GCACGATATC

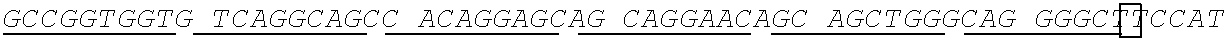

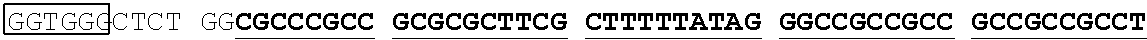

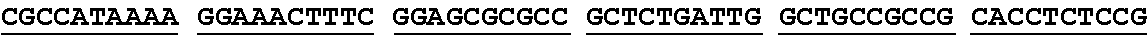

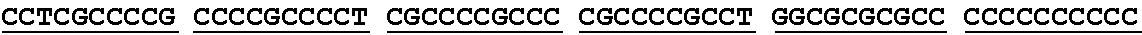

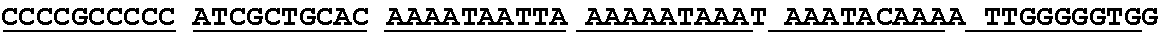

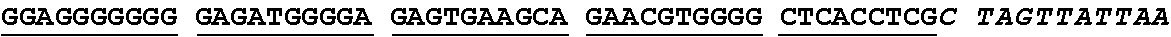

TAGTAATCAA TTACGGGGTC ATTAGTTCAT AGCCCATATA TGGAGTTCCG CGTTACATAA
CTTACGGTAA ATGGCCCGCC TGGCTGACCG CCCAACGACC CCCGCCCATT GACGTCAATA
ATGACGTATG TTCCCATAGT AACGCCAATA GGGACTTTCC ATTGACGTCA ATGGGTGGAG
TATTTACGGT AAACTGCCCA CTTGGCAGTA CATCAAGTGT ATCATATGCC AAGTACGCCC
CCTATTGACG TCAATGACGG TAAATGGCCC GCCTGGCATT ATGCCCAGTA CATGACCTTA

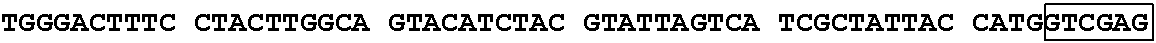

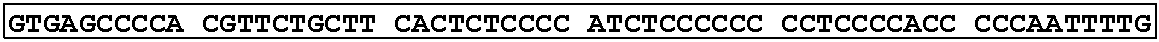

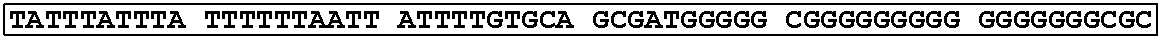

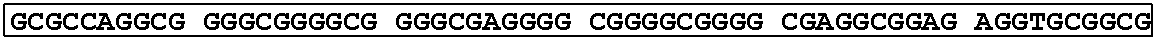

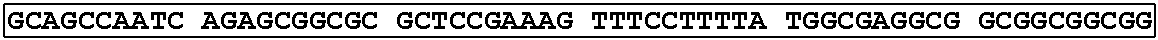

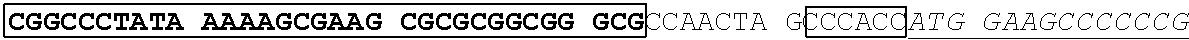

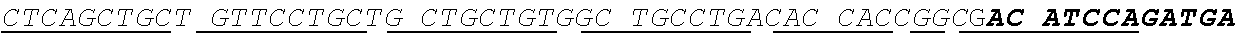

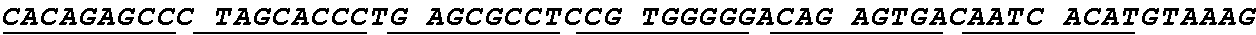

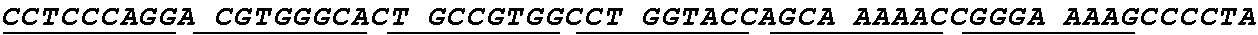

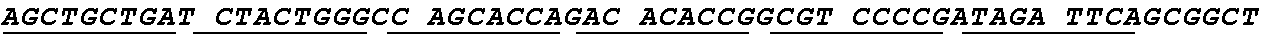

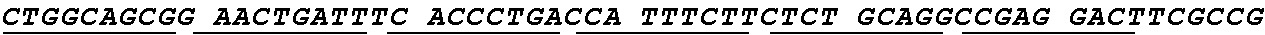

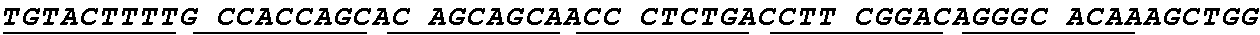

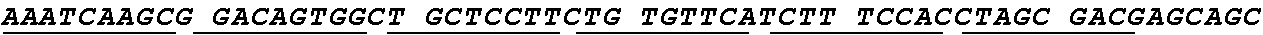

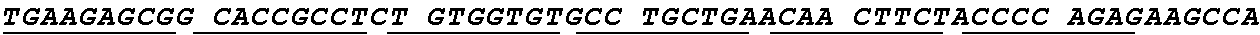

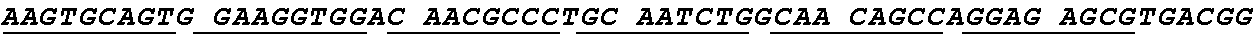

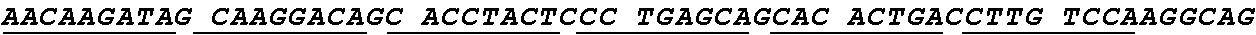

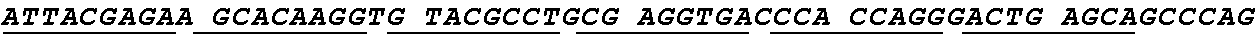

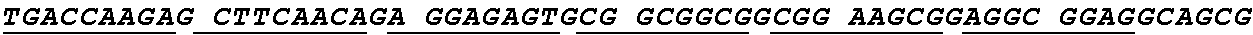

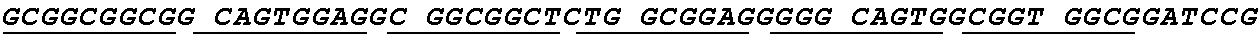

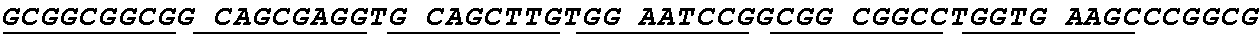

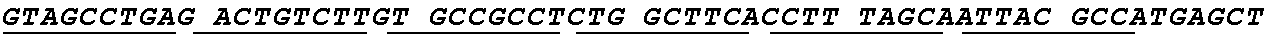

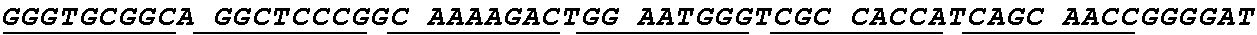

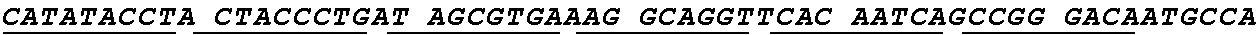

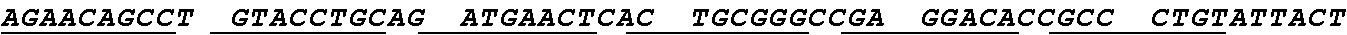

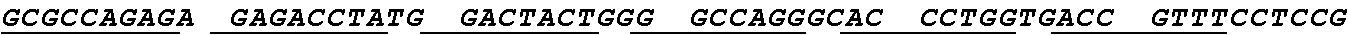

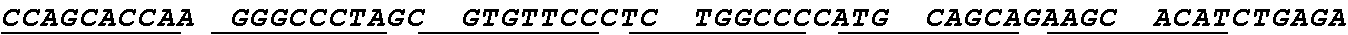

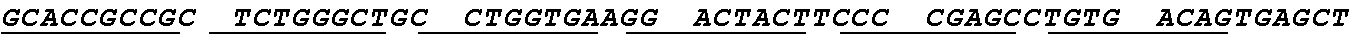

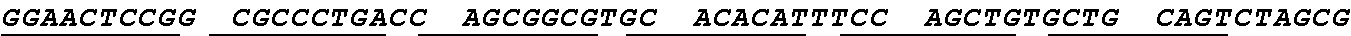

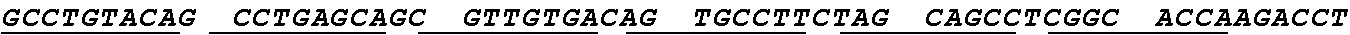

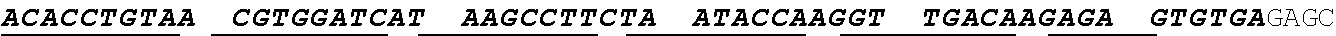

TCCCTGCAGG GTTTAAACCT GTGCCTTCTA GTTGCCAGCC ATCTGTTGTT TGCCCCTCCC
CCGTGCCTTC CTTGACCCTG GAAGGTGCCA CTCCCACTGT CCTTTCCTAA TAAAATGAGG
AAATTGCATC GCATTGTCTG AGTAGGTGTC ATTCTATTCT GGGGGGTGGG GTGGGGCAGG

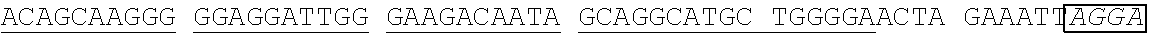

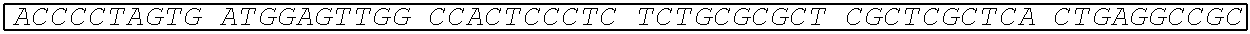

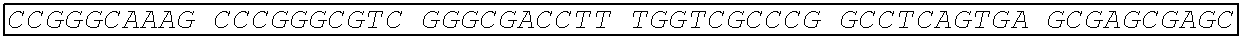

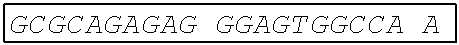

SEQ ID NO: 25 [αBb scFab - BiDir - αCls scFab] nucleic acid sequence
(Construct #10, FIG. 4A)
TCACACTCTC TTGTCAACCT TGGTATTAGA AGGCTTATGA TCCACGTTAC AGGTGTAGGT
CTTGGTGCCG AGGCTGCTAG AAGGCACTGT CACAACGCTG CTCAGGCTGT ACAGGCCGCT
AGACTGCAGC ACAGCTGGAA ATGTGTGCAC GCCGCTGGTC AGGGCGCCGG AGTTCCAGCT
CACTGTCACA GGCTCGGGGA AGTAGTCCTT CACCAGGCAG CCCAGAGCGG CGGTGCTCTC
AGATGTGCTT CTGCTGCATG GGGCCAGAGG GAACACGCTA GGGCCCTTGG TGCTGGCGGA
GGAAACGGTC ACCAGGGTGC CCTGGCCCCA GTAGTCCATA GGTCTCTCTC TGGCGCAGTA
ATACAGGGCG GTGTCCTCGG CCCGCAGTGA GTTCATCTGC AGGTACAGGC TGTTCTTGGC
ATTGTCCCGG CTGATTGTGA ACCTGCCTTT CACGCTATCA GGGTAGTAGG TATATGATCC
CCGGTTGCTG ATGGTGGCGA CCCATTCCAG TCTTTTGCCG GGAGCCTGCC GCACCCAGCT
CATGGCGTAA TTGCTAAAGG TGAAGCCAGA GGCGGCACAA GACAGTCTCA GGCTACCGCC
GGGCTTCACC AGGCCGCCGC CGGATTCCAC AAGCTGCACC TCGCTGCCGC CGCCGCCGGA
TCCGCCACCG CCACTGCCCC CTCCGCCAGA GCCGCCGCCT CCACTGCCGC CGCCGCCGCT
GCCTCCGCCT CCGCTTCCGC CGCCGCCGCA CTCTCCTCTG TTGAAGCTCT TGGTCACTGG
GCTGCTCAGT CCCTGGTGGG TCACCTCGCA GGCGTACACC TTGTGCTTCT CGTAATCTGC
CTTGGACAAG GTCAGTGTGC TGCTCAGGGA GTAGGTGCTG TCCTTGCTAT CTTGTTCCGT
CACGCTCTCC TGGCTGTTGC CAGATTGCAG GGCGTTGTCC ACCTTCCACT GCACTTTGGC
TTCTCTGGGG TAGAAGTIGT TCAGCAGGCA CACCACAGAG GCGGTGCCGC TCTTCAGCTG
CTCGTCGCTA GGTGGAAAGA TGAACACAGA AGGAGCAGCC ACTGTCCGCT TGATTTCCAG
CTTTGTGCCC TGTCCGAAGG TCAGAGGGTT GCTGCTGTGC TGGTGGCAAA AGTACACGGC
GAAGTCCTCG GCCTGCAGAG AAGAAATGGT CAGGGTGAAA TCAGTTCCGC TGCCAGAGCC
GCTGAATCTA TCGGGGACGC CGGTGTGTCT GGTGCTGGCC CAGTAGATCA GCAGCTTAGG
GGCTTTTCCC GGTTTTTGCT GGTACCAGGC CACGGCAGTG CCCACGTCCT GGGAGGCTTT
ACATGTGATT GTCACTCTGT CCCCCACGGA GGCGCTCAGG GTGCTAGGGC TCTGTGTCAT
CTGGATGTCG CCGGTGGTGT CAGGCAGCCA CAGCAGCAGC AGGAACAGCA GCTGAGCGGG
GGCTTCCATG GTGGGCTAGT TGGCGCCCGC CGCGCGCTTC GCTTTTTATA GGGCCGCCGC
CGCCGCCGCC TCGCCATAAA AGGAAACTTT CGGAGCGCGC CGCTCTGATT GGCTGCCGCC
GCACCTCTCC GCCTCGCCCC GCCCCGCCCC TCGCCCCGCC CCGCCCCGCC TGGCGCGCGC
CCCCCCCCCC CCCCCGCCCC CATCGCTGCA CAAAATAATT AAAAAATAAA TAAATACAAA
ATTGGGGGTG GGGAGGGGGG GGAGATGGGG AGAGTGAAGC AGAACGTGGG GCTCACCTCG
ACCATGGTAA TAGCGATGAC TAATACGTAG ATGTACTGCC AAGTAGGAAA GTCCCATAAG
GTCATGTACT GGGCATAATG CCAGGCGGGC CATTTACCGT CATTGACGTC AATAGGGGGC
GTACTTGGCA TATGATACAC TTGATGTACT GCCAAGTGGG CAGTTTACCG TAAATACTCC
ACCCATTGAC GTCAATGGAA AGTCCCTATT GGCGTTACTA TGGGAACATA CGTCATTATT
GACGTCAATG GGCGGGGGTC GTTGGGCGGT CAGCCAGGCG GGCCATTTAC CGTAAGTTAT
GTAACGCGGA ACTCCATATA TGGGCTATGA ACTAATGACC CCGTAATTGA TTACTATTAA
TAACTAGCGA GGTGAGCCCC ACGTTCTGCT TCACTCTCCC CATCTCCCCC CCCTCCCCAC
CCCCAATTTT GTATTTATTT ATTTTTTAAT TATTTTGTGC AGCGATGGGG GCGGGGGGGG
GGGGGGGGCG CGCGCCAGGC GGGGGGGGGC GGGGCGAGGG GCGGGGGGGG GCGAGGCGGA
GAGGTGCGGC GGCAGCCAAT CAGAGCGGCG CGCTCCGAAA GTTTCCTTTT ATGGCGAGGC
GGCGGCGGCG GCGGCCCTAT AAAAAGCGAA GCGCGCGGCG GGCGCCAGAG CCCACCATGG
AAGCCCCTGC CCAGCTGCTG TTCCTGCTGC TCCTGTGGCT GCCTGACACC ACCGGCGATA
TCGTGCTGAC GCAGAGCCCT GATAGCCTGG CCGTGAGCCT CGGCGAACGG GCCACAATCA
GCTGTAAAGC CTCTCAGAGC GTGGACTATG ACGGCGACAG CTACATGAAC TGGTACCAGC
AGAAACCCGG CCAGCCTCCA AAAATCCTGA TCTACGACGC CAGCAATCTG GAAAGCGGCA
TCCCCGCCAG ATTCAGCGGC AGCGGCTCTG GCACTGATTT CACCCTGACA ATTTCTTCTC
TGGAACCCGA GGATTTTGCC ATCTACTACT GCCAGCAGAG CAACGAGGAC CCCTGGACCT
TTGGAGGCGG CACCAAGGTG GAAATCAAGC GCACCGTGGC CGCCCCTTCT GTCTTTATCT
TCCCTCCAAG CGACGAGCAG CTGAAGAGCG GAACCGCCTC TGTGGTGTGC CTGCTGAATA
ACTTCTACCC CAGAGAGGCA AAGGTCCAAT GGAAAGTTGA CAACGCCCTG CAAAGCGGCA
ACAGCCAAGA GAGCGTGACC GAGCAGGACA GCAAGGACTC AACATACAGC CTGTCCAGCA
CCCTGACCTT GAGCAAGGCC GACTACGAGA AGCACAAGGT GTACGCCTGT GAAGTGACAC
ATCAGGGCCT GTCCTCTCCT GTGACCAAAA GCTTCAACCG GGGCGAGTGC GGCGGCGGCG
GCTCCGGCGG AGGAGGCAGC GGTGGCGGCG GCTCTGGGGG AGGCGGAAGC GGCGGAGGCG
GCAGCGGCGG AGGCGGCAGC GGCGGCGGCG GATCCCAGGT GCAGCTGGTG CAGAGCGGAG
CCGAGGTGAA AAAGCCTGGC GCTAGTGTTA AGCTGAGCTG CACCGCCAGC GGCTTCAATA
TCAAGGACGA TTACATCCAC TGGGTGAAGC AGGCCCCCGG CCAGGGACTG GAGTGGATCG
GCAGAATCGA CCCTGCCGAC GGCCACACAA AGTACGCCCC TAAGTTCCAG GTGAAAGTCA
CCATCACCGC CGATACCAGC ACCTCTACAG CTTATCTGGA ACTGAGCAGC CTTAGATCCG
AGGACACCGC TGTGTACTAC TGCGCCAGAT ACGGCTACGG CAGAGAAGTG TTCGACTACT
GGGGACAGGG CACAACAGTG ACCGTGTCGT CCGCCAGCAC AAAGGGCCCT AGCGTGTTCC
CACTGGCTCC TTGTAGCAGA AGTACCTCAG AGAGCACGGC TGCTCTGGGC TGCCTGGTCA
AGGATTATTT CCCTGAGCCT GTGACCGTGT CCTGGAACAG CGGAGCCCTG ACAAGCGGGG
TGCACACCTT CCCCGCCGTG CTGCAGAGCA GCGGCCTGTA CTCTCTGTCC TCTGTCGTGA
CAGTGCCTAG CTCTAGCCTG GGCACAAAGA CCTACACCTG CAACGTGGAC CACAAGCCCA
GCAACACCAA GGTGGATAAG CGGGTGTGA
SEQ ID NO: 26 - [αCls scFab - BiDir - αBb scFab-CM] nucleic acid
sequence (construct #21, FIG. 4B)
TCACACCCGC TTATCCACCT TGGTGTTGCT GGGCTTGTGG TCCACGTTGC AGGTGTAGGT
CTTTGTGCCC AGGCTAGAGC TAGGCACTGT CACGACAGAG GACAGAGAGT ACAGGCCGCT
GCTCTGCAGC ACGGCGGGGA AGGTGTGCAC CCCGCTTGTC AGGGCTCCGC TGTTCCAGGA
CACGGTCACA GGCTCAGGGA AATAATCCTT GACCAGGCAG CCCAGAGCAG CCGTGCTCTC
TGAGGTACTT CTGCTACAAG GAGCCAGTGG GAACACGCTA GGGCCCTTTG TGCTGGCGGA
CGACACGGTC ACTGTTGTGC CCTGTCCCCA GTAGTCGAAC ACTTCTCTGC CGTAGCCGTA
TCTGGCGCAG TAGTACACAG CGGTGTCCTC GGATCTAAGG CTGCTCAGTT CCAGATAAGC
TGTAGAGGTG CTGGTATCGG CGGTGATGGT GACTTTCACC TGGAACTTAG GGGCGTACTT
TGTGTGGCCG TCGGCAGGGT CGATTCTGCC GATCCACTCC AGTCCCTGGC CGGGGGCCTT
CTTCACCCAG TGGATGTAAT CGTCCTTGAT ATTGAAGCCG CTGGCGGTGC AGCTCAGCTT
AACACTAGCG CCAGGCTTTT TCACCTCGGC TCCGCTCTGC ACCAGCTGCA CCTGGGATCC
GCCGCCGCCG CTGCCGCCTC CGCCGCTGCC GCCTCCGCCG CTTCCGCCTC CCCCAGAGCC
GCCGCCACCG CTGCCTCCTC CGCCGGAGCC GCCGCCGCCG CACTCGCCCC GGTTGAAGCT
TTTGGTCACA GGAGAGGACA GGCCCTGATG TGTCACTTCA CAGGCGTACA CCTTGTGCTT
CTCGTAGTCG GCCTTGCTCA AGGTCAGGGT GCTGGACAGG CTGTATGTTG AGTCCTTGCT
GTCCTGCTCG GTCACGCTCT CTTGGCTGTT GCCGCTTTGC AGGGCGTTGT CAACTTTCCA
TTGGACCTTT GCCTCTCTGG GGTAGAAGTT ATTCAGCAGG CACACCACAG AGGCGGTTCC
GCTCTTCAGC TGCTCGTCGC TTGGAGGGAA GATAAAGACA GAAGGGGCGG CCACGGTGCG
CTTGATTTCC ACCTTGGTGC CGCCTCCAAA GGTCCAGGGG TCCTCGTTGC TCTGCTGGCA
GTAGTAGATG GCAAAATCCT CGGGTTCCAG AGAAGAAATT GTCAGGGTGA AATCAGTGCC
AGAGCCGCTG CCGCTGAATC TGGCGGGGAT GCCGCTTTCC AGATTGCTGG CGTCGTAGAT
CAGGATTTTT GGAGGCTGGC CGGGTTTCTC CTGGTACCAG TTCATGTAGC TGTCGCCGTC
ATAGTCCACG CTCTGAGAGG CTTTACAGCT GATTGTGGCC CGTTCGCCGA GGCTCACGGC
CAGGCTATCA GGGCTCTGCG TCAGCACGAT ATCGCCGGTG GTGTCAGGCA GCCACAGGAG
CAGCAGGAAC AGCAGCTGGG CAGGGGCTTC CATGGTGGGC TCTGGCGCCC GCCGCGCGCT
TCGCTTTTTA TAGGGCCGCC GCCGCCGCCG CCTCGCCATA AAAGGAAACT TTCGGAGCGC
GCCGCTCTGA TTGGCTGCCG CCGCACCTCT CCGCCTCGCC CCGCCCCGCC CCTCGCCCCG
CCCCGCCCCG CCTGGCGCGC GCCCCCCCCC CCCCCCCGCC CCCATCGCTG CACAAAATAA
TTAAAAAATA AATAAATACA AAATTGGGGG TGGGGAGGGG GGGGAGATGG GGAGAGTGAA
GCAGAACGTG GGGCTCACCT CGCTAGTTAT TAATAGTAAT CAATTACGGG GTCATTAGTT
CATAGCCCAT ATATGGAGTT CCGCGTTACA TAACTTACGG TAAATGGCCC GCCTGGCTGA
CCGCCCAACG ACCCCCGCCC ATTGACGTCA ATAATGACGT ATGTTCCCAT AGTAACGCCA
ATAGGGACTT TCCATTGACG TCAATGGGTG GAGTATTTAC GGTAAACTGC CCACTTGGCA
GTACATCAAG TGTATCATAT GCCAAGTACG CCCCCTATTG ACGTCAATGA CGGTAAATGG
CCCGCCTGGC ATTATGCCCA GTACATGACC TTATGGGACT TTCCTACTTG GCAGTACATC
TACGTATTAG TCATCGCTAT TACCATGGTC GAGGTGAGCC CCACGTTCTG CTTCACTCTC
CCCATCTCCC CCCCCTCCCC ACCCCCAATT TTGTATTTAT TTATTTTTTA ATTATTTTGT
GCAGCGATGG GGGCGGGGGG GGGGGGGGGG CGCGCGCCAG GCGGGGGGGG GCGGGGCGAG
GGGCGGGGCG GGGCGAGGCG GAGAGGTGCG GCGGCAGCCA ATCAGAGCGG CGCGCTCCGA
AAGTTTCCTT TTATGGCGAG GCGGCGGCGG CGGCGGCCCT ATAAAAAGCG AAGCGCGCGG
CGGGCGCCAA CTAGCCCACC ATGGAAGCCC CCGCTCAGCT GCTGTTCCTG CTGCTGCTGT
GGCTGCCTGA CACCACCGGC GACATCCAGA TGACACAGAG CCCTAGCACC CTGAGCGCCT
CCGTGGGGGA CAGAGTGACA ATCACATGTA AAGCCTCCCA GGACGTGGGC ACTGCCGTGG
CCTGGTACCA GAAAAAACCG GGAAAAGCCC CTAAGCTGCT GATCTACTGG GCCAGCACCA
GACACACCGG CGTCCCCGAT AGATTCAGCG GCTCTGGCAG CGGAACTGAT TTCACCCTGA
CCATTTCTTC TCTGCAGGCC GAGGACTTCG CCGTGTACTT TTGCCACCAG CACAGCAGCA
ACCCTCTGAC CTTCGGACAG GGCACAAAGC TGGAAATCAA GCGGACAGTG GCTGCTCCTT
CTGTGTTCAT CTTTCCACCT AGCGACGAGC AGCTGAAGAG CGGCACCGCC TCTGTGGTGT
GCCTGCTGAA CAACTTCTAC CCCAGAGAAG CCAAAGTGCA GTGGAAGGTG GACAACGCCC
TGCAATCTGG CAACAGCCAG GAGAGCGTGA CGGAACAAGA TAGCAAGGAC AGCACCTACT
CCCTGAGCAG CACACTGACC TTGTCCAAGG CAGATTACGA GAAGCACAAG GTGTACGCCT
GCGAGGTGAC CCACCAGGGA CTGAGCAGCC CAGTGACCAA GAGCTTCAAC AGAGGAGAGT
GCGGCGGCGG CGGAAGCGGA GGCGGAGGCA GCGGCGGCGG CGGCAGTGGA GGCGGCGGCT
CTGGCGGAGG GGGCAGTGGC GGTGGCGGAT CCGGCGGCGG CGGCAGCGAG GTGCAGCTTG
TGGAATCCGG CGGCGGCCTG GTGAAGCCCG GCGGTAGCCT GAGACTGTCT TGTGCCGCCT
CTGGCTTCAC CTTTAGCAAT TACGCCATGA GCTGGGTGCG GGAGGCTCCC GGCAAAAGAC
TGGAATGGGT CGCCACCATC AGCAACCGGG GATCATATAC CTACTACCCT GATAGCGTGA
AAGGCAGGTT CACAATCAGC CGGGACAATG CCAAGAACAG CCTGTACCTG CAGATGAACT
CACTGCGGGC CGAGGACACC GCCCTGTATT ACTGCGCCAG AGAGAGACCT ATGGACTACT
GGGGCCAGGG CACCCTGGTG ACCGTTTCCT CCGCCAGCAC CAAGGGCCCT AGCGTGTTCC
CTCTGGCCCC ATGCAGCAGA AGCACATCTG AGAGCACCGC CGCTCTGGGC TGCCTGGTGA
AGGACTACTT CCCCGAGCCT GTGACAGTGA GCTGGAACTC CGGCGCCCTG ACCAGCGGCG
TGCACACATT TCCAGCTGTG CTGCAGTCTA GCGGCCTGTA CAGCCTGAGC AGCGTTGTGA
CAGTGCCTTC TAGCAGCCTC GGCACCAAGA CCTACACCTG TAACGTGGAT CATAAGCCTT
CTAATACCAA GGTTGACAAG AGAGTGTGA
SEQ ID NO: 27 - [αBb scFab - BiDir - αCls scFab-CM] nucleic acid
sequence (construct #22, FIG. 4B)
TCACACTCTC TTGTCAACCT TGGTATTAGA AGGCTTATGA TCCACGTTAC AGGTGTAGGT
CTTGGTGCCG AGGCTGCTAG AAGGCACTGT CACAACGCTG CTCAGGCTGT ACAGGCCGCT
AGACTGCAGC ACAGCTGGAA ATGTGTGCAC GCCGCTGGTC AGGGCGCCGG AGTTCCAGCT
CACTGTCACA GGCTCGGGGA AGTAGTCCTT CACCAGGCAG CCCAGAGCGG CGGTGCTCTC
AGATGTGCTT CTGCTGCATG GGGCCAGAGG GAACACGCTA GGGCCCTTGG TGCTGGCGGA
GGAAACGGTC ACCAGGGTGC CCTGGCCCCA GTAGTCCATA GGTCTCTCTC TGGCGCAGTA
ATACAGGGCG GTGTCCTCGG CCCGCAGTGA GTTCATCTGC AGGTACAGGC TGTTCTTGGC
ATTGTCCCGG CTGATTGTGA ACCTGCCTTT CACGCTATCA GGGTAGTAGG TATATGATCC
CCGGTTGCTG ATGGTGGCGA CCCATTCCAG TCTTTTGCCG GGAGCCTCCC GCACCCAGCT
CATGGCGTAA TTGCTAAAGG TGAAGCCAGA GGCGGCACAA GACAGTCTCA GGCTACCGCC
GGGCTTCACC AGGCCGCCGC CGGATTCCAC AAGCTGCACC TCGCTGCCGC CGCCGCCGGA
TCCGCCACCG CCACTGCCCC CTCCGCCAGA GCCGCCGCCT CCACTGCCGC CGCCGCCGCT
GCCTCCGCCT CCGCTTCCGC CGCCGCCGCA CTCTCCTCTG TTGAAGCTCT TGGTCACTGG
GCTGCTCAGT CCCTGGTGGG TCACCTCGCA GGCGTACACC TTGTGCTTCT CGTAATCTGC
CTTGGACAAG GTCAGTGTGC TGCTCAGGGA GTAGGTGCTG TCCTTGCTAT CTTGTTCCGT
CACGCTCTCC TGGCTGTTGC CAGATTGCAG GGCGTTGTCC ACCTTCCACT GCACTTTGGC
TTCTCTGGGG TAGAAGTTGT TCAGCAGGCA CACCACAGAG GCGGTGCCGC TCTTCAGCTG
CTCGTCGCTA GGTGGAAAGA TGAACACAGA AGGAGCAGCC ACTGTCCGCT TGATTTCCAG
CTTTGTGCCC TGTCCGAAGG TCAGAGGGTT GCTGCTGTGC TGGTGGCAAA AGTACACGGC
GAAGTCCTCG GCCTGCAGAG AAGAAATGGT CAGGGTGAAA TCAGTTCCGC TGCCAGAGCC
GCTGAATCTA TCGGGGACGC CGGTGTGTCT GGTGCTGGCC CAGTAGATCA GCAGCTTAGG
GGCTTTTCCC GGTTTTTTCT GGTACCAGGC CACGGCAGTG CCCACGTCCT GGGAGGCTTT
ACATGTGATT GTCACTCTGT CCCCCACGGA GGCGCTCAGG GTGCTAGGGC TCTGTGTCAT
CTGGATGTCG CCGGTGGTGT CAGGCAGCCA CAGCAGCAGC AGGAACAGCA GCTGAGCGGG
GGCTTCCATG GTGGGCTAGT TGGCGCCCGC CGCGCGCTTC GCTTTTTATA GGGCCGCCGC
CGCCGCCGCC TCGCCATAAA AGGAAACTTT CGGAGCGCGC CGCTCTGATT GGCTGCCGCC
GCACCTCTCC GCCTCGCCCC GCCCCGCCCC TCGCCCCGCC CCGCCCCGCC TGGCGCGCGC
CCCCCCCCCC CCCCCGCCCC CATCGCTGCA CAAAATAATT AAAAAATAAA TAAATACAAA
ATTGGGGGTG GGGAGGGGGG GGAGATGGGG AGAGTGAAGC AGAACGTGGG GCTCACCTCG
ACCATGGTAA TAGCGATGAC TAATACGTAG ATGTACTGCC AAGTAGGAAA GTCCCATAAG
GTCATGTACT GGGCATAATG CCAGGCGGGC CATTTACCGT CATTGACGTC AATAGGGGGC
GTACTTGGCA TATGATACAC TTGATGTACT GCCAAGTGGG CAGTTTACCG TAAATACTCC
ACCCATTGAC GTCAATGGAA AGTCCCTATT GGCGTTACTA TGGGAACATA CGTCATTATT
GACGTCAATG GGCGGGGGTC GTTGGGCGGT CAGCCAGGCG GGCCATTTAC CGTAAGTTAT
GTAACGCGGA ACTCCATATA TGGGCTATGA ACTAATGACC CCGTAATTGA TTACTATTAA
TAACTAGCGA GGTGAGCCCC ACGTTCTGCT TCACTCTCCC CATCTCCCCC CCCTCCCCAC
CCCCAATTTT GTATTTATTT ATTTTTTAAT TATTTTGTGC AGCGATGGGG GCGGGGGGGG
GGGGGGGGCG CGCGCCAGGC GGGGGGGGGC GGGGCGAGGG GCGGGGGGGG GCGAGGCGGA
GAGGTGCGGC GGCAGCCAAT CAGAGCGGCG CGCTCCGAAA GTTTCCTTTT ATGGCGAGGC
GGCGGCGGCG GCGGCCCTAT AAAAAGCGAA GCGCGCGGCG GGCGCCAGAG CCCACCATGG
AAGCCCCTGC CCAGCTGCTG TTCCTGCTGC TCCTGTGGCT GCCTGACACC ACCGGCGATA
TCGTGCTGAC GCAGAGCCCT GATAGCCTGG CCGTGAGCCT CGGCGAACGG GCCACAATCA
GCTGTAAAGC CTCTCAGAGC GTGGACTATG ACGGCGACAG CTACATGAAC TGGTACCAGG
AGAAACCCGG CCAGCCTCCA AAAATCCTGA TCTACGACGC CAGCAATCTG GAAAGCGGCA
TCCCCGCCAG ATTCAGCGGC AGCGGCTCTG GCACTGATTT CACCCTGACA ATTTCTTCTC
TGGAACCCGA GGATTTTGCC ATCTACTACT GCCAGCAGAG CAACGAGGAC CCCTGGACCT
TTGGAGGCGG CACCAAGGTG GAAATCAAGC GCACCGTGGC CGCCCCTTCT GTCTTTATCT
TCCCTCCAAG CGACGAGCAG CTGAAGAGCG GAACCGCCTC TGTGGTGTGC CTGCTGAATA
ACTTCTACCC CAGAGAGGCA AAGGTCCAAT GGAAAGTTGA CAACGCCCTG CAAAGCGGCA
ACAGCCAAGA GAGCGTGACC GAGCAGGACA GCAAGGACTC AACATACAGC CTGTCCAGCA
CCCTGACCTT GAGCAAGGCC GACTACGAGA AGCACAAGGT GTACGCCTGT GAAGTGACAC
ATCAGGGCCT GTCCTCTCCT GTGACCAAAA GCTTCAACCG GGGCGAGTGC GGCGGCGGCG
GCTCCGGCGG AGGAGGCAGC GGTGGCGGCG GCTCTGGGGG AGGCGGAAGC GGCGGAGGCG
GCAGCGGCGG AGGCGGCAGC GGCGGCGGCG GATCCCAGGT GCAGCTGGTG CAGAGCGGAG
CCGAGGTGAA AAAGCCTGGC GCTAGTGTTA AGCTGAGCTG CACCGCCAGC GGCTTCAATA
TCAAGGACGA TTACATCCAC TGGGTGAAGA AGGCCCCCGG CCAGGGACTG GAGTGGATCG
GCAGAATCGA CCCTGCCGAC GGCCACACAA AGTACGCCCC TAAGTTCCAG GTGAAAGTCA
CCATCACCGC CGATACCAGC ACCTCTACAG CTTATCTGGA ACTGAGCAGC CTTAGATCCG
AGGACACCGC TGTGTACTAC TGCGCCAGAT ACGGCTACGG CAGAGAAGTG TTCGACTACT
GGGGACAGGG CACAACAGTG ACCGTGTCGT CCGCCAGCAC AAAGGGCCCT AGCGTGTTCC
CACTGGCTCC TTGTAGCAGA AGTACCTCAG AGAGCACGGC TGCTCTGGGC TGCCTGGTCA
AGGATTATTT CCCTGAGCCT GTGACCGTGT CCTGGAACAG CGGAGCCCTG ACAAGCGGGG
TGCACACCTT CCCCGCCGTG CTGCAGAGCA GCGGCCTGTA CTCTCTGTCC TCTGTCGTGA
CAGTGCCTAG CTCTAGCCTG GGCACAAAGA CCTACACCTG CAACGTGGAC CACAAGCCCA
GCAACACCAA GGTGGATAAG CGGGTGTGA
SEQ ID NO: 28 - BGH poly (A) signal sequence
CTGTGCCTTC TAGTTGCCAG CCATCTGTTG TTTGCCCCTC CCCCGTGCCT TCCTTGACCC
TGGAAGGTGC CACTCCCACT GTCCTTTCCT AATAAAATGA GGAAATTGCA TCGCATTGTC
TGAGTAGGTG TCATTCTATT CTGGGGGGTG GGGTGGGGCA GGACAGCAAG GGGGAGGATT
GGGAAGACAA TAGCAGGCAT GCTGGGGA
SEQ ID NO: 29 - Peptide Linker
(G₄S)ₙ, where n = 1, 2, 3, 4, 5, 6, 7, 8, 9, or 10.
SEQ ID NO: 30 - minCBA promoter (CMV enhancer underlined; CBA
promoter boldfaced and italicized; truncated chimeric intron:
boldfaced and underlined)
CTAGTTATTA ATAGTAATCA ATTACGGGGT CATTAGTTCA TAGCCCATAT ATGGAGTTCC
GCGTTACATA ACTTACGGTA AATGGCCCGC CTGGCTGACC GCCCAACGAC CCCCGCCCAT
TGACGTCAAT AATGACGTAT GTTCCCATAG TAACGCCAAT AGGGACTTTC CATTGACGTC
AATGGGTGGA GTATTTACGG TAAACTGCCC ACTTGGCAGT ACATCAAGTG TATCATATGC
CAAGTACGCC CCCTATTGAC GTCAATGACG GTAAATGGCC CGCCTGGCAT TATGCCCAGT
ACATGACCTT ATGGGACTTT CCTACTTGGC AGTACATCTA CGTATTAGTC ATCGCTATTA
CCATGGTCGA GGTGAGCCCC ACGTTCTGCT TCACTCTCCC CATCTCCCCC CCCTCCCCAC
CCCCAATTTT GTATTTATTT ATTTTTTAAT TATTTTGTGC AGCGATGGGG GCGGGGGGGG
GGGGGGGGCG CGCGCCAGGC GGGGCGGGGC GGGGCGAGGG GCGGGGGGGG GCGAGGCGGA
GAGGTGCGGC GGCAGCCAAT CAGAGCGGCG CGCTCCGAAA GTTTCCTTTT ATGGCGAGGC

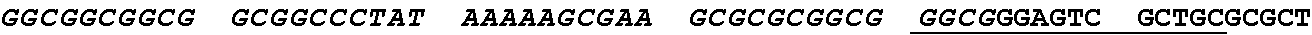

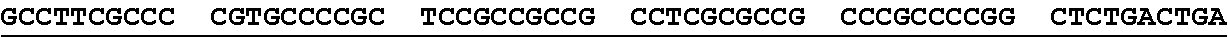

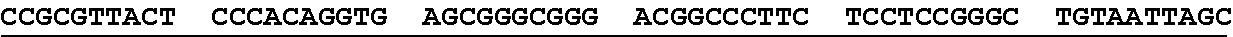

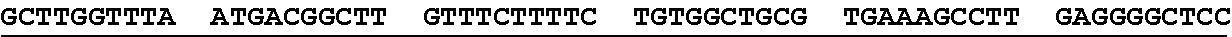

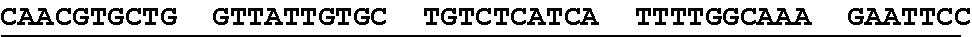

SEQ ID NO: 31 ITR-BiD-CBA-anti Cls scFab (5′ ITR boldfaced; bGH polyA
signal underlined; reverse complement of aCls scFab coding sequence
italicized; IgG kappa signal coding sequence italicized and
underlined; Kozak sequence boxed; CBA promoter (reverse) bolded and
underlined; CMV enhancer boldfaced and italicized; CBA promoter
boxed and underlined; and 3′ ITR boxed and italicized)
TTGGCCACTC CCTCTCTGCG CGCTCGCTCG CTCACTGAGG CCGCCCGGGC AAAGCCCGGG
CGTCGGGCGA CCTTTGGTCG CCCGGCCTCA GTGAGCGAGC GAGCGCGCAG AGAGGGAGTG
GCCAACTCCA TCACTAGGGG TTCCTTACAA TTCTAGTTCC CCAGCATGCC TGCTATTGTC
TTCCCAATCC TCCCCCTTGC TGTCCTGCCC CACCCCACCC CCCAGAATAG AATGACACCT
ACTCAGACAA TGCGATGCAA TTTCCTCATT TTATTAGGAA AGGACAGTGG GAGTGGCACC
TTCCAGGGTC AAGGAAGGCA CGGGGGAGGG GCAAACAACA GATGGCTGGC AACTAGAAGG
CACAGGTTTA AACCCTGCAG GGAGCTCTCA CACCCGCTTA TCCACCTTGG TGTTGCTGGG
CTTGTGGTCC ACGTTGCAGG TGTAGGTCTT TGTGCCCAGG CTAGAGCTAG GCACTGTCAC
GACAGAGGAC AGAGAGTACA GGCCGCTGCT CTGCAGCACG GCGGGGAAGG TGTGCACCCC
GCTTGTCAGG GCTCCGCTGT TCCAGGACAC GGTCACAGGC TCAGGGAAAT AATCCTTGAC
CAGGCAGCCC AGAGCAGCCG TGCTCTCTGA GGTACTTCTG CTACAAGGAG CCAGTGGGAA
CACGCTAGGG CCCTTTGTGC TGGCGGACGA CACGGTCACT GTTGTGCCCT GTCCCCAGTA
GTCGAACACT TCTCTGCCGT AGCCGTATCT GGCGCAGTAG TACACAGCGG TGTCCTCGGA
TCTAAGGCTG CTCAGTTCCA GATAAGCTGT AGAGGTGCTG GTATCGGCGG TGATGGTGAC
TTTCACCTGG AACTTAGGGG CGTACTTTGT GTGGCCGTCG GCAGGGTCGA TTCTGCCGAT
CCACTCCAGT CCCTGGCCGG GGGCCTGCTT CACCCAGTGG ATGTAATCGT CCTTGATATT
GAAGCCGCTG GCGGTGCAGC TCAGCTTAAC ACTAGCGCCA GGCTTTTTCA CCTCGGCTCC
GCTCTGCACC AGCTGCACCT GGGATCCGCC GCCGCCGCTG CCGCCTCCGC CGCTGCCGCC
TCCGCCGCTT CCGCCTCCCC CAGAGCCGCC GCCACCGCTG CCTCCTCCGC CGGAGCCGCC
GCCGCCGCAC TCGCCCCGGT TGAAGCTTTT GGTCACAGGA GAGGACAGGC CCTGATGTGT
CACTTCACAG GCGTACACCT TGTGCTTCTC GTAGTCGGCC TTGCTCAAGG TCAGGGTGCT
GGACAGGCTG TATGTTGAGT CCTTGCTGTC CTGCTCGGTC ACGCTCTCTT GGCTGTTGCC
GCTTTGCAGG GCGTTGTCAA CTTTCCATTG GACCTTTGCC TCTCTGGGGT AGAAGTTATT
CAGCAGGCAC ACCACAGAGG CGGTTCCGCT CTTCAGCTGC TCGTCGCTTG GAGGGAAGAT
AAAGACAGAA GGGGCGGCCA CGGTGCGCTT GATTTCCACC TTGGTGCCGC CTCCAAAGGT
CCAGGGGTCC TCGTTGCTCT GCTGGCAGTA GTAGATGGCA AAATCCTCGG GTTCCAGAGA
AGAAATTGTC AGGGTGAAAT CAGTGCCAGA GCCGCTGCCG CTGAATCTGG CGGGGATGCC
GCTTTCCAGA TTGCTGGCGT CGTAGATCAG GATTTTTGGA GGCTGGCCGG GTTTCTGCTG
GTACCAGTTC ATGTAGCTGT CGCCGTCATA GTCCACGCTC TGAGAGGCTT TACAGCTGAT
TGTGGCCCGT TCGCCGAGGC TCACGGCCAG GCTATCAGGG CTCTGCGTCA GCACGATATC
GCCGGTGGTG TCAGGCAGCC ACAGGAGCAG CAGGAACAGC AGCTGGGCAG GGGCTTCCAT

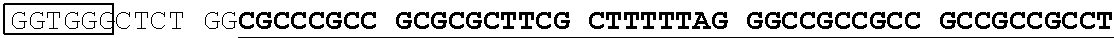

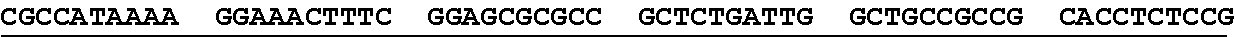

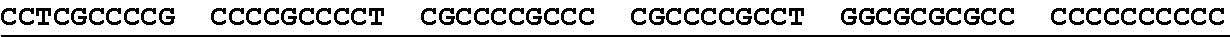

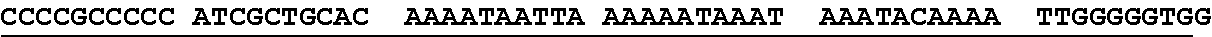

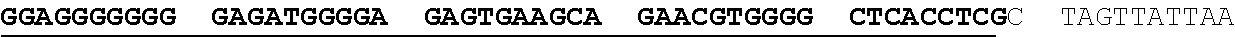

TAGTAATCAA TTACGGGGTC ATTAGTTCAT AGCCCATATA TGGAGTTCCG CGTTACATAA
CTTACGGTAA ATGGCCCGCC TGGCTGACCG CCCAACGACC CCCGCCCATT GACGTCAATA
ATGACGTATG TTCCCATAGT AACGCCAATA GGGACTTTCC ATTGACGTCA ATGGGTGGAG
TATTTACGGT AAACTGCCCA CTTGGCAGTA CATCAAGTGT ATCATATGCC AAGTACGCCC
CCTATTGACG TCAATGACGG TAAATGGCCC GCCTGGCATT ATGCCCAGTA CATGACCTTA

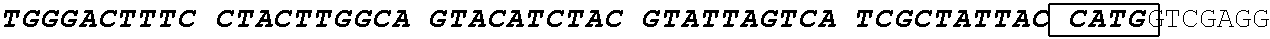

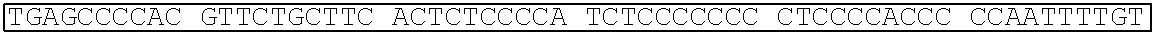

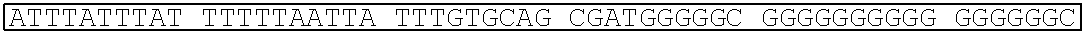

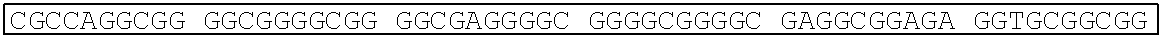

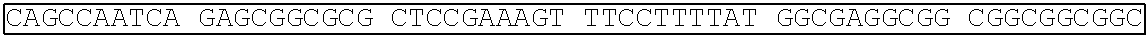

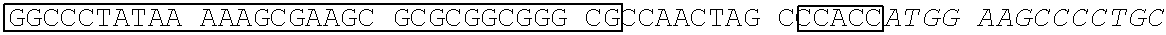

CCAGCTGCTG TTCCTGCTGC TCCTGTGGCT GCCTGACACC ACCGGCGATA TCGTGCTGAC
GCAGAGCCCT GATAGCCTGG CCGTGAGCCT CGGCGAACGG GCCACAATCA GCTGTAAAGC
CTCTCAGAGC GTGGACTATG ACGGCGACAG CTACATGAAC TGGTACCAGC AGAAACCCGG
CCAGCCTCCA AAAATCCTGA TCTACGACGC CAGCAATCTG GAAAGCGGCA TCCCCGCCAG
ATTCAGCGGC AGCGGCTCTG GCACTGATTT CACCCTGACA ATTTCTTCTC TGGAACCCGA
GGATTTTGCC ATCTACTACT GCCAGCAGAG CAACGAGGAC CCCTGGACCT TTGGAGGCGG
CACCAAGGTG GAAATCAAGC GCACCGTGGC CGCCCCTTCT GTCTTTATCT TCCCTCCAAG
CGACGAGCAG CTGAAGAGCG GAACCGCCTC TGTGGTGTGC CTGCTGAATA ACTTCTACCC
CAGAGAGGCA AAGGTCCAAT GGAAAGTTGA CAACGCCCTG CAAAGCGGCA ACAGCCAAGA
GAGCGTGACC GAGCAGGACA GCAAGGACTC AACATACAGC CTGTCCAGCA CCCTGACCTT
GAGCAAGGCC GACTACGAGA AGCACAAGGT GTACGCCTGT GAAGTGACAC ATCAGGGCCT
GTCCTCTCCT GTGACCAAAA GCTTCAACCG GGGCGAGTGC GGCGGCGGCG GCTCCGGCGG
AGGAGGCAGC GGTGGCGGCG GCTCTGGGGG AGGCGGAAGC GGCGGAGGCG GCAGCGGCGG
AGGCGGCAGC GGCGGCGGCG GATCCCAGGT GCAGCTGGTG CAGAGCGGAG CCGAGGTGAA
AAAGCCTGGC GCTAGTGTTA AGCTGAGCTG CACCGCCAGC GGCTTCAATA TCAAGGACGA
TTACATCCAC TGGGTGAAGC AGGCCCCCGG CCAGGGACTG GAGTGGATCG GCAGAATCGA
CCCTGCCGAC GGCCACACAA AGTACGCCCC TAAGTTCCAG GTGAAAGTCA CCATCACCGC
CGATACCAGC ACCTCTACAG CTTATCTGGA ACTGAGCAGC CTTAGATCCG AGGACACCGC
TGTGTACTAC TGCGCCAGAT ACGGCTACGG CAGAGAAGTG TTCGACTACT GGGGACAGGG
CACAACAGTG ACCGTGTCGT CCGCCAGCAC AAAGGGCCCT AGCGTGTTCC CACTGGCTCC
TTGTAGCAGA AGTACCTCAG AGAGCACGGC TGCTCTGGGC TGCCTGGTCA AGGATTATTT
CCCTGAGCCT GTGACCGTGT CCTGGAACAG CGGAGCCCTG ACAAGCGGGG TGCACACCTT
CCCCGCCGTG CTGCAGAGCA GCGGCCTGTA CTCTCTGTCC TCTGTCGTGA CAGTGCCTAG
CTCTAGCCTG GGCACAAAGA CCTACACCTG CAACGTGGAC CACAAGCCCA GCAACACCAA
GGTGGATAAG CGGGTGTGAG AGCTCCCTGC AGGGTTTAAA CCTGTGCCTT CTAGTTGCCA
GCCATCTGTT GTTTGCCCCT CCCCCGTGCC TTCCTTGACC CTGGAAGGTG CCACTCCCAC
TGTCCTTTCC TAATAAAATG AGGAAATTGC ATCGCATTGT CTGAGTAGGT GTCATTCTAT
TCTGGGGGGT GGGGTGGGGC AGGACAGCAA GGGGGAGGAT TGGGAAGACA ATAGCAGGCA
TGCTGGGGAA CTAGAAATTA GGAACCCCTA GTGATGGAGT TGGCCACTCC CTCTCTGCGC

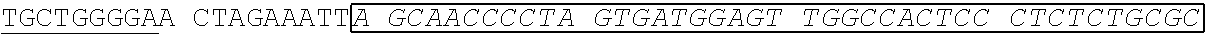

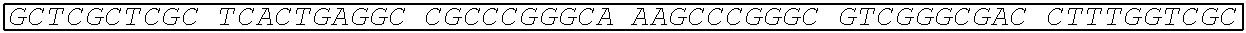

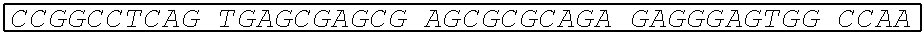

SEQ ID NO: 32 ITR-BiD-CBA-anti Bb scFab (5′ ITR boldfaced; bGH polyA
signal underlined; reverse complement of Bb scFab coding sequence
italicized; IgG kappa signal coding sequence italicized and
underlined; Kozak sequence boxed; CBA promoter (reverse) bolded and
underlined; CMV enhancer boldfaced and italicized; CBA promoter
boxed and underlined; and 3′ ITR boxed and italicized)
TTGGCCACTC CCTCTCTGCG CGCTCGCTCG CTCACTGAGG CCGCCCGGGC AAAGCCCGGG
CGTCGGGCGA CCTTTGGTCG CCCGGCCTCA GTGAGCGAGC GAGCGCGCAG AGAGGGAGTG
GCCAACTCCA TCACTAGGGG TTCCTTACAA TTCTAGTTCC CCAGCATGCC TGCTATTGTC
TTCCCAATCC TCCCCCTTGC TGTCCTGCCC CACCCCACCC CCCAGAATAG AATGACACCT
ACTCAGACAA TGCGATGCAA TTTCCTCATT TTATTAGGAA AGGACAGTGG GAGTGGCACC
TTCCAGGGTC AAGGAAGGCA CGGGGGAGGG GCAAACAACA GATGGCTGGC AACTAGAAGG
CACAGGTTTA AACCCTGCAG GGAGCTCGTC ACACTCTCTT GTCAACCTTG GTATTAGAAG
GCTTATGATC CACGTTACAG GTGTAGGTCT TGGTGCCGAG GCTGCTAGAA GGCACTGTCA
CAACGCTGCT CAGGCTGTAC AGGCCGCTAG ACTGCAGCAC AGCTGGAAAT GTGTGCACGC
CGCTGGTCAG GGCGCCGGAG TTCCAGCTCA CTGTCACAGG CTCGGGGAAG TAGTCCTTCA
CCAGGCAGCC CAGAGCGGCG GTGCTCTCAG ATGTGCTTCT GCTGCATGGG GCCAGAGGGA
ACACGCTAGG GCCCTTGGTG CTGGCGGAGG AAACGGTCAC CAGGGTGCCC TGGCCCCAGT
AGTCCATAGG TCTCTCTCTG GCGCAGTAAT ACAGGGCGGT GTCCTCGGCC CGCAGTGAGT
TCATCTGCAG GTACAGGCTG TTCTTGGCAT TGTCCCGGCT GATTGTGAAC CTGCCTTTCA
CGCTATCAGG GTAGTAGGTA TATGATCCCC GGTTGCTGAT GGTGGCGACC CATTCCAGTC
TTTTGCCGGG AGCCTGCCGC ACCCAGCTCA TGGCGTAATT GCTAAAGGTG AAGCCAGAGG
CGGCACAAGA CAGTCTCAGG CTACCGCCGG GCTTCACCAG GCCGCCGCCG GATTCCACAA
GCTGCACCTC GCTGCCGCCG CCGCCGGATC CGCCACCGCC ACTGCCCCCT CCGCCAGAGC
CGCCGCCTCC ACTGCCGCCG CCGCCGCTGC CTCCGCCTCC GCTTCCGCCG CCGCCGCACT
CTCCTCTGTT GAAGCTCTTG GTCACTGGGC TGCTCAGTCC CTGGTGGGTC ACCTCGCAGG
CGTACACCTT GTGCTTCTCG TAATCTGCCT TGGACAAGGT CAGTGTGCTG CTCAGGGAGT
AGGTGCTGTC CTTGCTATCT TGTTCCGTCA CGCTCTCCTG GCTGTTGCCA GATTGCAGGG
CGTTGTCCAC CTTCCACTGC ACTTTGGCTT CTCTGGGGTA GAAGTTGTTC AGCAGGCACA
CCACAGAGGC GGTGCCGCTC TTCAGCTGCT CGTCGCTAGG TGGAAAGATG AACACAGAAG
GAGCAGCCAC TGTCCGCTTG ATTTCCAGCT TTGTGCCCTG TCCGAAGGTC AGAGGGTTGC
TGCTGTGCTG GTGGCAAAAG TACACGGCGA AGTCCTCGGC CTGCAGAGAA GAAATGGTCA
GGGTGAAATC AGTTCCGCTG CCAGAGCCGC TGAATCTATC GGGGACGCCG GTGTGTCTGG
TGCTGGCCCA GTAGATCAGC AGCTTAGGGG CTTTTCCCGG TTTTTGCTGG TACCAGGCCA
CGGCAGTGCC CACGTCCTGG GAGGCTTTAC ATGTGATTGT CACTCTGTCC CCCACGGAGG
CGCTCAGGGT GCTAGGGCTC TGTGTCATCT GGATGTCGCC GGTGGTGTCA GGCAGCCACA

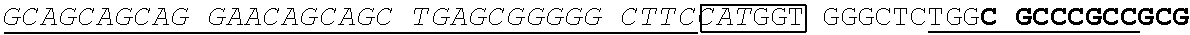

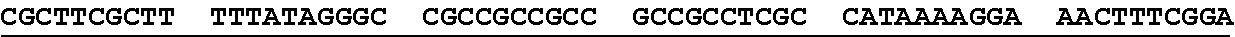

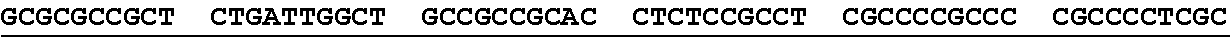

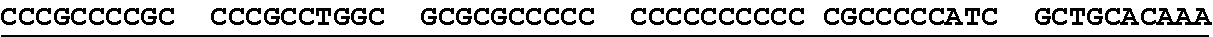

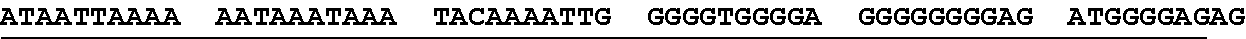

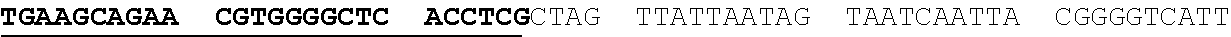

AGTTCATAGC CCATATATGG AGTTCCGCGT TACATAACTT ACGGTAAATG GCCCGCCTGG
CTGACCGCCC AACGACCCCC GCCCATTGAC GTCAATAATG ACGTATGTTC CCATAGTAAC
GCCAATAGGG ACTTTCCATT GACGTCAATG GGTGGAGTAT TTACGGTAAA CTGCCCACTT
GGCAGTACAT CAAGTGTATC ATATGCCAAG TACGCCCCCT ATTGACGTCA ATGACGGTAA
ATGGCCCGCC TGGCATTATG CCCAGTACAT GACCTTATGG GACTTTCCTA CTTGGCAGTA

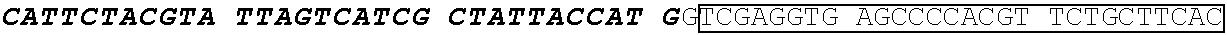

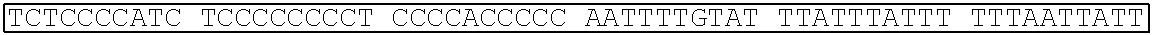

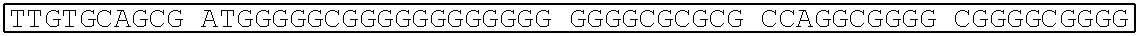

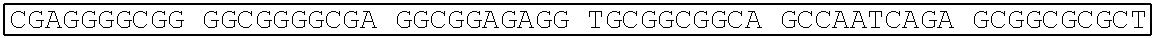

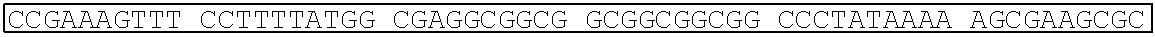

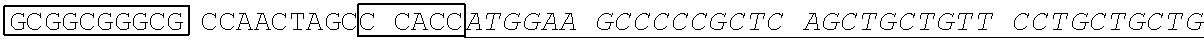

CTGTGGCTGC CTGACACCAC CGGCGACATC CAGATGACAC AGAGCCCTAG CACCCTGAGC
GCCTCCGTGG GGGACAGAGT GACAATCACA TGTAAAGCCT CCCAGGACGT GGGCACTGCC
GTGGCCTGGT ACCAGCAAAA ACCGGGAAAA GCCCCTAAGC TGCTGATCTA CTGGGCCAGC
ACCAGACACA CCGGCGTCCC CGATAGATTC AGCGGCTCTG GCAGCGGAAC TGATTTCACC
CTGACCATTT CTTCTCTGCA GGCCGAGGAC TTCGCCGTGT ACTTTTGCCA CCAGCACAGC
AGCAACCCTC TGACCTTCGG ACAGGGCACA AAGCTGGAAA TCAAGCGGAC AGTGGCTGCT
CCTTCTGTGT TCATCTTTCC ACCTAGCGAC GAGCAGCTGA AGAGCGGCAC CGCCTCTGTG
GTGTGCCTGC TGAACAACTT CTACCCCAGA GAAGCCAAAG TGCAGTGGAA GGTGGACAAC
GCCCTGCAAT CTGGCAACAG CCAGGAGAGC GTGACGGAAC AAGATAGCAA GGACAGCACC
TACTCCCTGA GCAGCACACT GACCTTGTCC AAGGCAGATT ACGAGAAGCA CAAGGTGTAC
GCCTGCGAGG TGACCCACCA GGGACTGAGC AGCCCAGTGA CCAAGAGCTT CAACAGAGGA
GAGTGCGGCG GCGGCGGAAG CGGAGGCGGA GGCAGCGGCG GCGGCGGCAG TGGAGGCGGC
GGCTCTGGCG GAGGGGGCAG TGGCGGTGGC GGATCCGGCG GCGGCGGCAG CGAGGTGCAG
CTTGTGGAAT CCGGCGGCGG CCTGGTGAAG CCCGGCGGTA GCCTGAGACT GTCTTGTGCC
GCCTCTGGCT TCACCTTTAG CAATTACGCC ATGAGCTGGG TGCGGCAGGC TCCCGGCAAA
AGACTGGAAT GGGTCGCCAC CATCAGCAAC CGGGGATCAT ATACCTACTA CCCTGATAGC
GTGAAAGGCA GGTTCACAAT CAGCCGGGAC AATGCCAAGA ACAGCCTGTA CCTGCAGATG
AACTCACTGC GGGCCGAGGA CACCGCCCTG TATTACTGCG CCAGAGAGAG ACCTATGGAC
TACTGGGGCC AGGGCACCCT GGTGACCGTT TCCTCCGCCA GCACCAAGGG CCCTAGCGTG
TTCCCTCTGG CCCCATGCAG CAGAAGCACA TCTGAGAGCA CCGCCGCTCT GGGCTGCCTG
GTGAAGGACT ACTTCCCCGA GCCTGTGACA GTGAGCTGGA ACTCCGGCGC CCTGACCAGC
GGCGTGCACA CATTTCCAGC TGTGCTGCAG TCTAGCGGCC TGTACAGCCT GAGCAGCGTT
GTGACAGTGC CTTCTAGCAG CCTCGGCACC AAGACCTACA CCTGTAACGT GGATCATAAG
CCTTCTAATA CCAAGGTTGA CAAGAGAGTG TGAGAGCTCC CTGCAGGGTT TAAACCTGTG
CCTTCTAGTT GCCAGCCATC TGTTGTTTGC CCCTCCCCCG TGCCTTCCTT GACCCTGGAA
GGTGCCACTC CCACTGTCCT TTCCTAATAA AATGAGGAAA TTGCATCGCA TTGTCTGAGT
AGGTGTCATT CTATTCTGGG GGGTGGGGTG GGGCAGGACA GCAAGGGGGA GGATTGGGAA

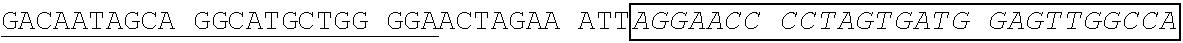

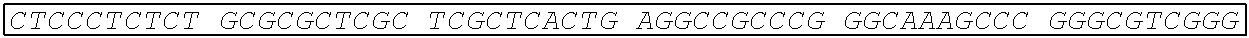

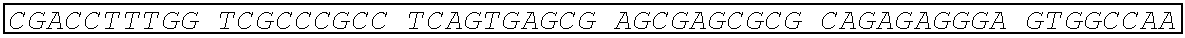

SEQ ID NO: 33 - ITR-BiD-CBA-aCls scFab-hSyn-Bb scFab - (5′ ITR
boldfaced; bGH polyA signal underlined; reverse complement of xC1s
scFab coding sequence italicized; IgG kappa signal coding sequence
italicized and underlined; Kozak sequence boxed; CBA
promoter (reverse) bolded and underlined; CMV enhancer boldfaced and
italicized; Synapsin promoter boxed and underlined; anti-Bb scFab
boldfaced, italicized, and underlined; and 3′ ITR boxed and
italicized)
TTGGCCACTC CCTCTCTGCG CGCTCGCTCG CTCACTGAGG CCGCCCGGGC AAAGCCCGGG
CGTCGGGCGA CCTTTGGTCG CCCGGCCTCA GTGAGCGAGC GAGCGCGCAG AGAGGGAGTG
GCCAACTCCA TCACTAGGGG TTCCTTACAA TTCTAGTTCC CCAGCATGCC TGCTATTGTC
TTCCCAATCC TCCCCCTTGC TGTCCTGCCC CACCCCACCC CCCAGAATAG AATGACACCT
ACTCAGACAA TGCGATGCAA TTTCCTCATT TTATTAGGAA AGGACAGTGG GAGTGGCACC
TTCCAGGGTC AAGGAAGGCA CGGGGGAGGG GCAAACAACA GATGGCTGGC AACTAGAAGG
CACAGGTTTA AACCCTGCAG GGAGCTCTCA CACCCGCTTA TCCACCTTGG TGTTGCTGGG
CTTGTGGTCC ACGTTGCAGG TGTAGGTCTT TGTGCCCAGG CTAGAGCTAG GCACTGTCAC
GACAGAGGAC AGAGAGTACA GGCCGCTGCT CTGCAGCACG GCGGGGAAGG TGTGCACCCC
GCTTGTCAGG GCTCCGCTGT TCCAGGACAC GGTCACAGGC TCAGGGAAAT AATCCTTGAC
CAGGCAGCCC AGAGCAGCCG TGCTCTCTGA GGTACTTCTG CTACAAGGAG CCAGTGGGAA
CACGCTAGGG CCCTTTGTGC TGGCGGACGA CACGGTCACT GTTGTGCCCT GTCCCCAGTA
GTCGAACACT TCTCTGCCGT AGCCGTATCT GGCGCAGTAG TACACAGCGG TGTCCTCGGA
TCTAAGGCTG CTCAGTTCCA GATAAGCTGT AGAGGTGCTG GTATCGGCGG TGATGGTGAC
TTTCACCTGG AACTTAGGGG CGTACTTTGT GTGGCCGTCG GCAGGGTCGA TTCTGCCGAT
CCACTCCAGT CCCTGGCCGG GGGCCTGCTT CACCCAGTGG ATGTAATCGT CCTTGATATT
GAAGCCGCTG GCGGTGCAGC TCAGCTTAAC ACTAGCGCCA GGCTTTTTCA CCTCGGCTCC
GCTCTGCACC AGCTGCACCT GGGATCCGCC GCCGCCGCTG CCGCCTCCGC CGCTGCCGCC
TCCGCCGCTT CCGCCTCCCC CAGAGCCGCC GCCACCGCTG CCTCCTCCGC CGGAGCCGCC
GCCGCCGCAC TCGCCCCGGT TGAAGCTTTT GGTCACAGGA GAGGACAGGC CCTGATGTGT
CACTTCACAG GCGTACACCT TGTGCTTCTC GTAGTCGGCC TTGCTCAAGG TCAGGGTGCT
GGACAGGCTG TATGTTGAGT CCTTGCTGTC CTGCTCGGTC ACGCTCTCTT GGCTGTTGCC
GCTTTGCAGG GCGTTGTCAA CTTTCCATTG GACCTTTGCC TCTCTGGGGT AGAAGTTATT
CAGCAGGCAC ACCACAGAGG CGGTTCCGCT CTTCAGCTGC TCGTCGCTTG GAGGGAAGAT
AAAGACAGAA GGGGCGGCCA CGGTGCGCTT GATTTCCACC TTGGTGCCGC CTCCAAAGGT
CCAGGGGTCC TCGTTGCTCT GCTGGCAGTA GTAGATGGCA AAATCCTCGG GTTCCAGAGA
AGAAATTGTC AGGGTGAAAT CAGTGCCAGA GCCGCTGCCG CTGAATCTGG CGGGGATGCC
GCTTTCCAGA TTGCTGGCGT CGTAGATCAG GATTTTTGGA GGCTGGCCGG GTTTCTGCTG
GTACCAGTTC ATGTAGCTGT CGCCGTCATA GTCCACGCTC TGAGAGGCTT TACAGCTGAT
TGTGGCCCGT TCGCCGAGGC TCACGGCCAG GCTATCAGGG CTCTGCGTCA GCACGATATC
GCCGGTGGTG TCAGGCAGCC ACAGGAGCAG CAGGAACAGC AGCTGGGCAG GGGCTTCCAT

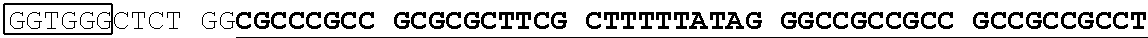

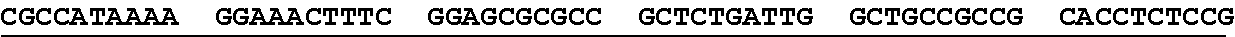

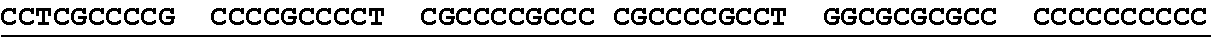

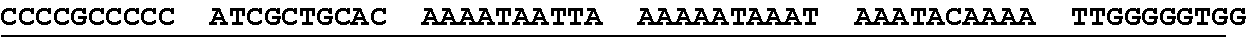

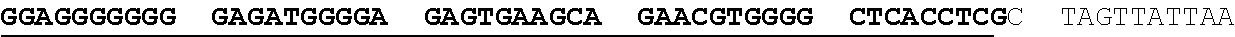

TAGTAATCAA TTACGGGGTC ATTAGTTCAT AGCCCATATA TGGAGTTCCG CGTTACATAA
CTTACGGTAA ATGGCCCGCC TGGCTGACCG CCCAACGACC CCCGCCCATT GACGTCAATA
ATGACGTATG TTCCCATAGT AACGCCAATA GGGACTTTCC ATTGACGTCA ATGGGTGGAG
TATTTACGGT AAACTGCCCA CTTGGCAGTA CATCAAGTGT ATCATATGCC AAGTACGCCC
CCTATTGACG TCAATGACGG TAAATGGCCC GCCTGGCATT ATGCCCAGTA CATGACCTTA

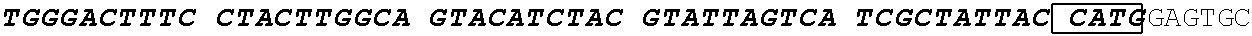

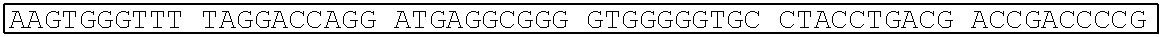

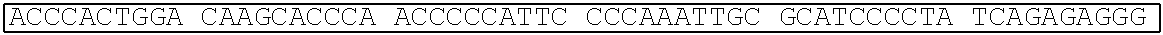

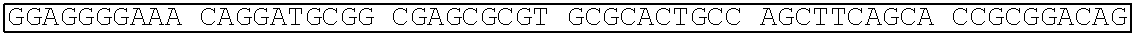

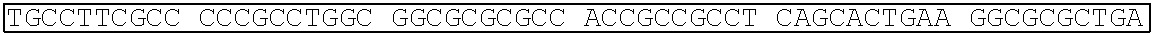

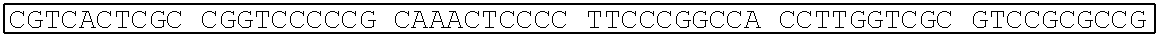

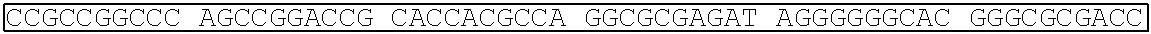

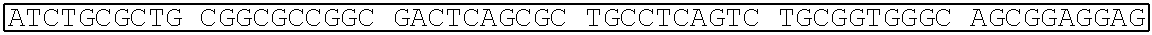

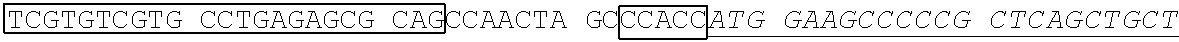

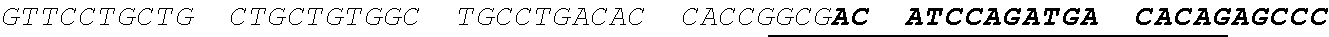

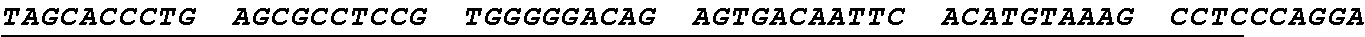

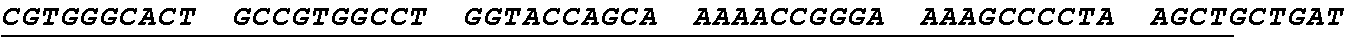

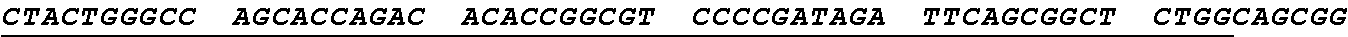

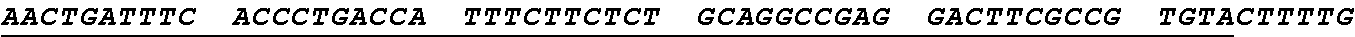

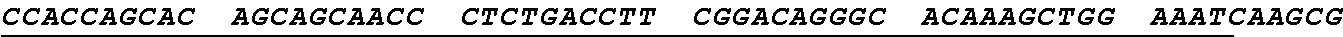

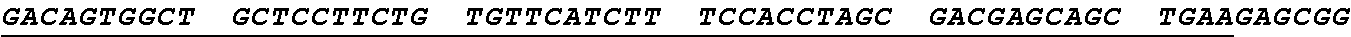

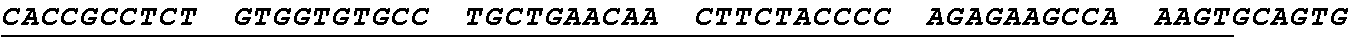

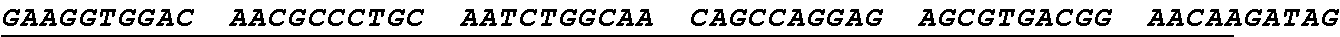

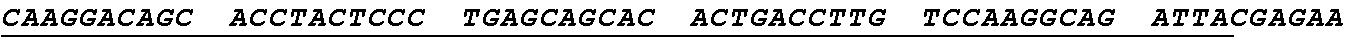

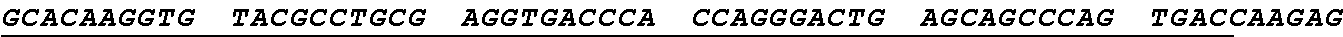

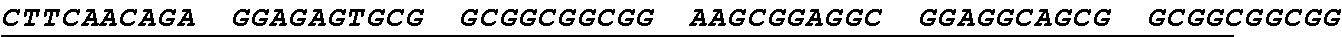

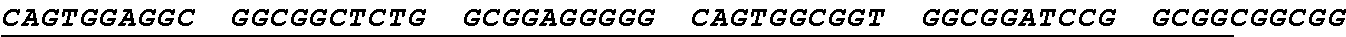

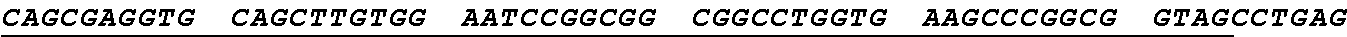

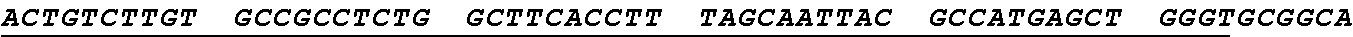

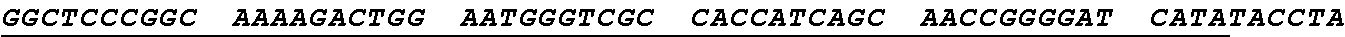

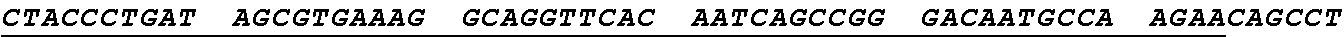

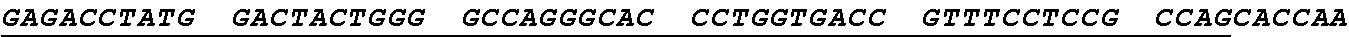

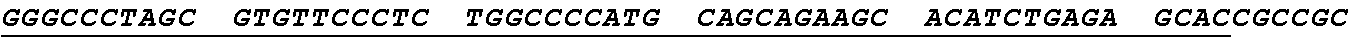

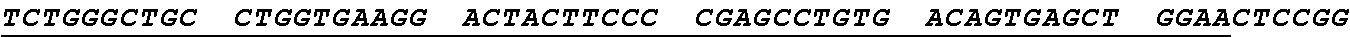

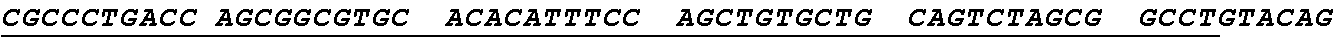

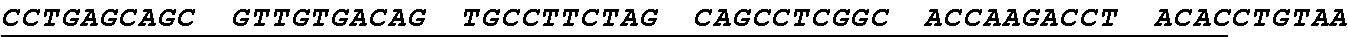

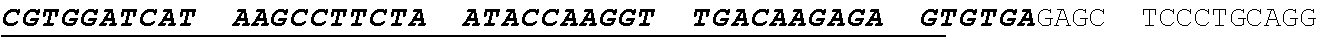

GTTTAAACCT GTGCCTTCTA GTTGCCAGCC ATCTGTTGTT TGCCCCTCCC CCGTGCCTTC
CTTGACCCTG GAAGGTGCCA CTCCCACTGT CCTTTCCTAA TAAAATGAGG AAATTGCATC
GCATTGTCTG AGTAGGTGTC ATTCTATTCT GGGGGGTGGG GTGGGGCAGG ACAGCAAGGG

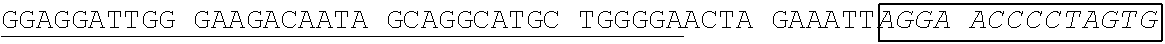

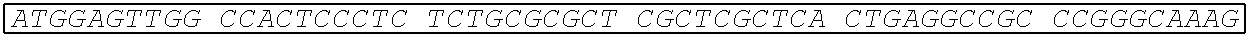

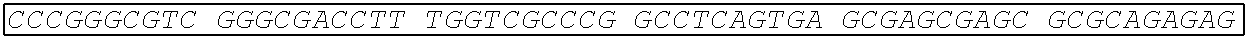

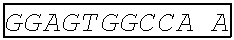

1. A method of producing a recombinant adeno-associated virus (AAV) composition comprising a first AAV having a self-complementary recombinant genome comprising a first transgene and a second AAV having a self-complementary recombinant genome comprising a second transgene, the method comprising:
introducing into a host cell an exogenous DNA encoding a template AAV genome comprising an expression cassette flanked by AAV inverted terminal repeats (ITRs), wherein the expression cassette comprises the first and second transgenes placed in opposite directions and separated by a bidirectional promoter, and wherein the bidirectional promoter comprises palindromic sequences,
culturing the host cell under conditions for AAV replication, wherein a recombinant AAV composition is produced, and
optionally isolating the recombinant AAV composition produced from the host cell.

2. The method of claim 1, wherein the host cell is a mammalian cell engineered to express an AAV Rep gene and an AAV Cap gene.

3. The method of claim 2, wherein the exogenous DNA, the AAV Rep gene and the AAV Cap gene are stably integrated into the host cell genome and the AAV replication is initiated by infecting the host cell with an adenovirus or a herpes simplex 1 virus.

4. The method of claim 2, wherein the exogenous DNA, the AAV Rep gene, the AAV Cap gene, and adenoviral helper genes are introduced into the host cell by transient transfection, wherein the adenoviral helper genes comprise E4, E2a, and VA genes.

5. The method of claim 1, wherein the ratio of the first AAV to the second AAV in the composition is about 0.5:1 to about 2:1, optionally about 1:1.

6. The method of claim 1, wherein the bidirectional promoter is a minCBA promoter comprising a pair of chicken β-actin (CBA) promoters placed in opposite direction and separated by a CMV enhancer.

7. The method of claim 6, wherein the CMV enhancer comprises SEQ ID NO:8, or a nucleic acid sequence at least 85% identical thereto.

8. The method of claim 6, wherein the bidirectional promoter comprises SEQ ID NO:1 or 30, or a nucleic acid sequence at least 85% identical thereto.

9. The method of claim 1, wherein the AAV ITRs are AAV2 ITRs.

10. The method of claim 1, wherein the first transgene comprises a portion of a full-length gene and the second transgene comprises the remainder of the full-length gene, wherein the full-length gene is 4.5 to 9 kb long, and said portion and said remainder each are no longer than 4.8 kb,
(a) wherein the first transgene comprises a splice donor at the 3′ end of its coding region and the second transgene comprises a splice acceptor at the 5′ end of its coding region, and wherein the splice donor and the splice acceptor promote generation of an RNA transcript of the full-length gene upon co-delivery of the first and second AAVs into a target cell; or
(b) wherein the 3′ coding region of the first transgene and the 5′ coding region of the second transgene overlap by 10 or more nucleotides, and wherein the overlap region promotes generation of an RNA transcript of the full-length gene upon co-delivery of the first and second AAVs into a target cell.

11. The method of claim 1, wherein the first and second transgenes each code for a different therapeutic protein.

12. The method of claim 11, wherein the first transgene encodes an anti-C1s antibody fragment, optionally an scFv or an scFab, and the second transgene encodes an anti-Bb antibody fragment, optionally an scFv or an scFab.

13. The method of claim 1, wherein the first and second transgenes each code for the same therapeutic protein, optionally wherein the two transgenes are identical and the first AAV and the second AAV are identical.

14. The method of claim 13, wherein the first transgene and second transgene both encode an anti-C1s antibody fragment, optionally an scFv or an scFab, or the first transgene and second transgenes both encode an anti-Bb antibody fragment, optionally an scFv or an scFab.

15. The method of claim 12, wherein the anti-C1s antibody comprises
HCDR1-3 and LCDR1-3 comprising SEQ ID NOs:14-19, respectively;
V_{H} and V_{L} comprising SEQ ID NOs:20 and 21, respectively; or
SEQ ID NO:22 or 23.

16. The method of claim 12, wherein the anti-Bb antibody comprises
HCDR1-3 and LCDR1-3 comprising SEQ ID NOs:4-9, respectively;
V_{H} and V_{L} comprising SEQ ID NOs:10 and 11, respectively; or
SEQ ID NO:12 or 13.

17. The method of claim 12, wherein the template AAV genome comprises SEQ ID NO:24, 25, 26, or 27.

18. The method of claim 14, wherein the template AAV genome comprises SEQ ID NO:31 or 32.

19. The method of claim 1, wherein the host cell is a mammalian cell, optionally a 293, HeLa, or A549 cell.

20. A recombinant AAV composition produced by the method of claim 1.

21. A method of treating a disease in a human patient in need thereof, comprising delivering the recombinant AAV composition of claim 20 to the patient.

22. A recombinant AAV composition produced by the method of claim 12.

23. A method of treating dry age-related macular degeneration (AMD) in a human patient in need thereof, comprising delivering the recombinant AAV composition of claim 22 to a diseased eye of the patient.


SEQUENCE LISTING (33 sequences)

SEQ ID NO: 1 - DNA, 921 bp, synthetic construct
  source 1..921: mol_type = other DNA
cgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 2 - DNA, 278 bp, synthetic construct
  source 1..278: mol_type = other DNA
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 3 - DNA, 304 bp, synthetic construct
  source 1..304: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 4 - protein, 5 aa, synthetic construct
DDYIH

SEQ ID NO: 5 - protein, 17 aa, synthetic construct
RIDPADGHTKYAPKFQV

SEQ ID NO: 6 - protein, 10 aa, synthetic construct
YGYGREVFDY

SEQ ID NO: 7 - protein, 15 aa, synthetic construct
KASQSVDYDGDSYMN

SEQ ID NO: 8 - protein, 7 aa, synthetic construct
DASNLES

SEQ ID NO: 9 - protein, 9 aa, synthetic construct
QQSNEDPWT

SEQ ID NO: 10 - protein, 119 aa, synthetic construct
QVQLVQSGAEVKKPGASVKLSCTASGFNIKDDYIHWVKQAPGQGLEWIGRIDPADGHTKYAPKFQVKVTITADTSTSTAYLELSSLRSEDTAVYYCARYGYGREVFDYWGQGTTVTVSS

SEQ ID NO: 11 - protein, 111 aa, synthetic construct
DIVLTQSPDSLAVSLGERATISCKASQSVDYDGDSYMNWYQQKPGQPPKILIYDASNLESGIPARFSGSGSGTDFTLTISSLEPEDFAIYYCQQSNEDPWTFGGGTKVEIK

SEQ ID NO: 12 - protein, 470 aa, synthetic construct
DIVLTQSPDSLAVSLGERATISCKASQSVDYDGDSYMNWYQQKPGQPPKILIYDASNLESGIPARFSGSGSGTDFTLTISSLEPEDFAIYYCQQSNEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSQVQLVQSGAEVKKPGASVKLSCTASGFNIKDDYIHWVKQAPGQGLEWIGRIDPADGHTKYAPKFQVKVTITADTSTSTAYLELSSLRSEDTAVYYCARYGYGREVFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 13 - protein, 490 aa, synthetic construct
MEAPAQLLFLLLLWLPDTTGDIVLTQSPDSLAVSLGERATISCKASQSVDYDGDSYMNWYQEKPGQPPKILIYDASNLESGIPARFSGSGSGTDFTLTISSLEPEDFAIYYCQQSNEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSQVQLVQSGAEVKKPGASVKLSCTASGFNIKDDYIHWVKKAPGQGLEWIGRIDPADGHTKYAPKFQVKVTITADTSTSTAYLELSSLRSEDTAVYYCARYGYGREVFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 14 - protein, 5 aa, synthetic construct
NYAMS

SEQ ID NO: 15 - protein, 17 aa, synthetic construct
TISNRGSYTYYPDSVKG

SEQ ID NO: 16 - protein, 6 aa, synthetic construct
ERPMDY

SEQ ID NO: 17 - protein, 11 aa, synthetic construct
KASQDVGTAVA

SEQ ID NO: 18 - protein, 7 aa, synthetic construct
WASTRHT

SEQ ID NO: 19 - protein, 9 aa, synthetic construct
HQHSSNPLT

SEQ ID NO: 20 - protein, 115 aa, synthetic construct
EVQLVESGGGLVKPGGSLRLSCAASGFTFSNYAMSWVRQAPGKRLEWVATISNRGSYTYYPDSVKGRFTISRDNAKNSLYLQMNSLRAEDTALYYCARERPMDYWGQGTLVTVSS

SEQ ID NO: 21 - protein, 107 aa, synthetic construct
DIQMTQSPSTLSASVGDRVTITCKASQDVGTAVAWYQQKPGKAPKLLIYWASTRHTGVPDRFSGSGSGTDFTLTISSLQAEDFAVYFCHQHSSNPLTFGQGTKLEIK

SEQ ID NO: 22 - protein, 462 aa, synthetic construct
DIQMTQSPSTLSASVGDRVTITCKASQDVGTAVAWYQQKPGKAPKLLIYWASTRHTGVPDRFSGSGSGTDFTLTISSLQAEDFAVYFCHQHSSNPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSEVQLVESGGGLVKPGGSLRLSCAASGFTFSNYAMSWVRQAPGKRLEWVATISNRGSYTYYPDSVKGRFTISRDNAKNSLYLQMNSLRAEDTALYYCARERPMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 23 - protein, 482 aa, synthetic construct
MEAPAQLLFLLLLWLPDTTGDIQMTQSPSTLSASVGDRVTITCKASQDVGTAVAWYQKKPGKAPKLLIYWASTRHTGVPDRFSGSGSGTDFTLTISSLQAEDFAVYFCHQHSSNPLTFGQGTKLEIKRTVAAPAVFIFPPSDEQLKSGTASVVCLLKNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSEVQLVESGGGLVKPGGSLRLSCAASGFTFSNYAMSWVREAPGKRLEWVATISNRGSYTYYPDSVKGRFTISRDNAKNSLYLQMNSLRAEDTALYYCARERPMDYWGQGTLVTVSSASTKGPSVFPLAPCSRSTSESTAALGCLVKDYFPEPVTVSWNSGALTSGVHTFPAVLQSSGLYSLSSVVEVPSSSLGTKTYTCNVDHKPSNTKVDKRV

SEQ ID NO: 24 - DNA, 4641 bp, synthetic construct
  source 1..4641: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccttacaattctagttccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacaggtttaaaccctgcagggagctctcacacccgcttatccaccttggtgttgctgggcttgtggtccacgttgcaggtgtaggtctttgtgcccaggctagagctaggcactgtcacgacagaggacagagagtacaggccgctgctctgcagcacggcggggaaggtgtgcaccccgcttgtcagggctccgctgttccaggacacggtcacaggctcagggaaataatccttgaccaggcagcccagagcagccgtgctctctgaggtacttctgctacaaggagccagtgggaacacgctagggccctttgtgctggcggacgacacggtcactgttgtgccctgtccccagtagtcgaacacttctctgccgtagccgtatctggcgcagtagtacacagcggtgtcctcggatctaaggctgctcagttccagataagctgtagaggtgctggtatcggcggtgatggtgactttcacctggaacttaggggcgtactttgtgtggccgtcggcagggtcgattctgccgatccactccagtccctggccgggggcctgcttcacccagtggatgtaatcgtccttgatattgaagccgctggcggtgcagctcagcttaacactagcgccaggctttttcacctcggctccgctctgcaccagctgcacctgggatccgccgccgccgctgccgcctccgccgctgccgcctccgccgcttccgcctcccccagagccgccgccaccgctgcctcctccgccggagccgccgccgccgcactcgccccggttgaagcttttggtcacaggagaggacaggccctgatgtgtcacttcacaggcgtacaccttgtgcttctcgtagtcggccttgctcaaggtcagggtgctggacaggctgtatgttgagtccttgctgtcctgctcggtcacgctctcttggctgttgccgctttgcagggcgttgtcaactttccattggacctttgcctctctggggtagaagttattcagcaggcacaccacagaggcggttccgctcttcagctgctcgtcgcttggagggaagataaagacagaaggggcggccacggtgcgcttgatttccaccttggtgccgcctccaaaggtccaggggtcctcgttgctctgctggcagtagtagatggcaaaatcctcgggttccagagaagaaattgtcagggtgaaatcagtgccagagccgctgccgctgaatctggcggggatgccgctttccagattgctggcgtcgtagatcaggatttttggaggctggccgggtttctgctggtaccagttcatgtagctgtcgccgtcatagtccacgctctgagaggctttacagctgattgtggcccgttcgccgaggctcacggccaggctatcagggctctgcgtcagcacgatatcgccggtggtgtcaggcagccacaggagcagcaggaacagcagctgggcaggggcttccatggtgggctctggcgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgccaactagcccaccatggaagcccccgctcagctgctgttcctgctgctgctgtggctgcctgacaccaccggcgacatccagatgacacagagccctagcaccctgagcgcctccgtgggggacagagtgacaatcacatgtaaagcctcccaggacgtgggcactgccgtggcctggtaccagcaaaaaccgggaaaagcccctaagctgctgatctactgggccagcaccagacacaccggcgtccccgatagattcagcggctctggcagcggaactgatttcaccctgaccatttcttctctgcaggccgaggacttcgccgtgtacttttgccaccagcacagcagcaaccctctgaccttcggacagggcacaaagctggaaatcaagcggacagtggctgctccttctgtgttcatctttccacctagcgacgagcagctgaagagcggcaccgcctctgtggtgtgcctgctgaacaacttctaccccagagaagccaaagtgcagtggaaggtggacaacgccctgcaatctggcaacagccaggagagcgtgacggaacaagatagcaaggacagcacctactccctgagcagcacactgaccttgtccaaggcagattacgagaagcacaaggtgtacgcctgcgaggtgacccaccagggactgagcagcccagtgaccaagagcttcaacagaggagagtgcggcggcggcggaagcggaggcggaggcagcggcggcggcggcagtggaggcggcggctctggcggagggggcagtggcggtggcggatccggcggcggcggcagcgaggtgcagcttgtggaatccggcggcggcctggtgaagcccggcggtagcctgagactgtcttgtgccgcctctggcttcacctttagcaattacgccatgagctgggtgcggcaggctcccggcaaaagactggaatgggtcgccaccatcagcaaccggggatcatatacctactaccctgatagcgtgaaaggcaggttcacaatcagccgggacaatgccaagaacagcctgtacctgcagatgaactcactgcgggccgaggacaccgccctgtattactgcgccagagagagacctatggactactggggccagggcaccctggtgaccgtttcctccgccagcaccaagggccctagcgtgttccctctggccccatgcagcagaagcacatctgagagcaccgccgctctgggctgcctggtgaaggactacttccccgagcctgtgacagtgagctggaactccggcgccctgaccagcggcgtgcacacatttccagctgtgctgcagtctagcggcctgtacagcctgagcagcgttgtgacagtgccttctagcagcctcggcaccaagacctacacctgtaacgtggatcataagccttctaataccaaggttgacaagagagtgtgagagctccctgcagggtttaaacctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaactagaaattaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 25 - DNA, 3869 bp, synthetic construct
  source 1..3869: mol_type = other DNA
tcacactctcttgtcaaccttggtattagaaggcttatgatccacgttacaggtgtaggtcttggtgccgaggctgctagaaggcactgtcacaacgctgctcaggctgtacaggccgctagactgcagcacagctggaaatgtgtgcacgccgctggtcagggcgccggagttccagctcactgtcacaggctcggggaagtagtccttcaccaggcagcccagagcggcggtgctctcagatgtgcttctgctgcatggggccagagggaacacgctagggcccttggtgctggcggaggaaacggtcaccagggtgccctggccccagtagtccataggtctctctctggcgcagtaatacagggcggtgtcctcggcccgcagtgagttcatctgcaggtacaggctgttcttggcattgtcccggctgattgtgaacctgcctttcacgctatcagggtagtaggtatatgatccccggttgctgatggtggcgacccattccagtcttttgccgggagcctgccgcacccagctcatggcgtaattgctaaaggtgaagccagaggcggcacaagacagtctcaggctaccgccgggcttcaccaggccgccgccggattccacaagctgcacctcgctgccgccgccgccggatccgccaccgccactgccccctccgccagagccgccgcctccactgccgccgccgccgctgcctccgcctccgcttccgccgccgccgcactctcctctgttgaagctcttggtcactgggctgctcagtccctggtgggtcacctcgcaggcgtacaccttgtgcttctcgtaatctgccttggacaaggtcagtgtgctgctcagggagtaggtgctgtccttgctatcttgttccgtcacgctctcctggctgttgccagattgcagggcgttgtccaccttccactgcactttggcttctctggggtagaagttgttcagcaggcacaccacagaggcggtgccgctcttcagctgctcgtcgctaggtggaaagatgaacacagaaggagcagccactgtccgcttgatttccagctttgtgccctgtccgaaggtcagagggttgctgctgtgctggtggcaaaagtacacggcgaagtcctcggcctgcagagaagaaatggtcagggtgaaatcagttccgctgccagagccgctgaatctatcggggacgccggtgtgtctggtgctggcccagtagatcagcagcttaggggcttttcccggtttttgctggtaccaggccacggcagtgcccacgtcctgggaggctttacatgtgattgtcactctgtcccccacggaggcgctcagggtgctagggctctgtgtcatctggatgtcgccggtggtgtcaggcagccacagcagcagcaggaacagcagctgagcgggggcttccatggtgggctagttggcgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgccagagcccaccatggaagcccctgcccagctgctgttcctgctgctcctgtggctgcctgacaccaccggcgatatcgtgctgacgcagagccctgatagcctggccgtgagcctcggcgaacgggccacaatcagctgtaaagcctctcagagcgtggactatgacggcgacagctacatgaactggtaccagcagaaacccggccagcctccaaaaatcctgatctacgacgccagcaatctggaaagcggcatccccgccagattcagcggcagcggctctggcactgatttcaccctgacaatttcttctctggaacccgaggattttgccatctactactgccagcagagcaacgaggacccctggacctttggaggcggcaccaaggtggaaatcaagcgcaccgtggccgccccttctgtctttatcttccctccaagcgacgagcagctgaagagcggaaccgcctctgtggtgtgcctgctgaataacttctaccccagagaggcaaaggtccaatggaaagttgacaacgccctgcaaagcggcaacagccaagagagcgtgaccgagcaggacagcaaggactcaacatacagcctgtccagcaccctgaccttgagcaaggccgactacgagaagcacaaggtgtacgcctgtgaagtgacacatcagggcctgtcctctcctgtgaccaaaagcttcaaccggggcgagtgcggcggcggcggctccggcggaggaggcagcggtggcggcggctctgggggaggcggaagcggcggaggcggcagcggcggaggcggcagcggcggcggcggatcccaggtgcagctggtgcagagcggagccgaggtgaaaaagcctggcgctagtgttaagctgagctgcaccgccagcggcttcaatatcaaggacgattacatccactgggtgaagcaggcccccggccagggactggagtggatcggcagaatcgaccctgccgacggccacacaaagtacgcccctaagttccaggtgaaagtcaccatcaccgccgataccagcacctctacagcttatctggaactgagcagccttagatccgaggacaccgctgtgtactactgcgccagatacggctacggcagagaagtgttcgactactggggacagggcacaacagtgaccgtgtcgtccgccagcacaaagggccctagcgtgttcccactggctccttgtagcagaagtacctcagagagcacggctgctctgggctgcctggtcaaggattatttccctgagcctgtgaccgtgtcctggaacagcggagccctgacaagcggggtgcacaccttccccgccgtgctgcagagcagcggcctgtactctctgtcctctgtcgtgacagtgcctagctctagcctgggcacaaagacctacacctgcaacgtggaccacaagcccagcaacaccaaggtggataagcgggtgtga

SEQ ID NO: 26 - DNA, 3869 bp, synthetic construct
  source 1..3869: mol_type = other DNA
tcacacccgcttatccaccttggtgttgctgggcttgtggtccacgttgcaggtgtaggtctttgtgcccaggctagagctaggcactgtcacgacagaggacagagagtacaggccgctgctctgcagcacggcggggaaggtgtgcaccccgcttgtcagggctccgctgttccaggacacggtcacaggctcagggaaataatccttgaccaggcagcccagagcagccgtgctctctgaggtacttctgctacaaggagccagtgggaacacgctagggccctttgtgctggcggacgacacggtcactgttgtgccctgtccccagtagtcgaacacttctctgccgtagccgtatctggcgcagtagtacacagcggtgtcctcggatctaaggctgctcagttccagataagctgtagaggtgctggtatcggcggtgatggtgactttcacctggaacttaggggcgtactttgtgtggccgtcggcagggtcgattctgccgatccactccagtccctggccgggggccttcttcacccagtggatgtaatcgtccttgatattgaagccgctggcggtgcagctcagcttaacactagcgccaggctttttcacctcggctccgctctgcaccagctgcacctgggatccgccgccgccgctgccgcctccgccgctgccgcctccgccgcttccgcctcccccagagccgccgccaccgctgcctcctccgccggagccgccgccgccgcactcgccccggttgaagcttttggtcacaggagaggacaggccctgatgtgtcacttcacaggcgtacaccttgtgcttctcgtagtcggccttgctcaaggtcagggtgctggacaggctgtatgttgagtccttgctgtcctgctcggtcacgctctcttggctgttgccgctttgcagggcgttgtcaactttccattggacctttgcctctctggggtagaagttattcagcaggcacaccacagaggcggttccgctcttcagctgctcgtcgcttggagggaagataaagacagaaggggcggccacggtgcgcttgatttccaccttggtgccgcctccaaaggtccaggggtcctcgttgctctgctggcagtagtagatggcaaaatcctcgggttccagagaagaaattgtcagggtgaaatcagtgccagagccgctgccgctgaatctggcggggatgccgctttccagattgctggcgtcgtagatcaggatttttggaggctggccgggtttctcctggtaccagttcatgtagctgtcgccgtcatagtccacgctctgagaggctttacagctgattgtggcccgttcgccgaggctcacggccaggctatcagggctctgcgtcagcacgatatcgccggtggtgtcaggcagccacaggagcagcaggaacagcagctgggcaggggcttccatggtgggctctggcgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgccaactagcccaccatggaagcccccgctcagctgctgttcctgctgctgctgtggctgcctgacaccaccggcgacatccagatgacacagagccctagcaccctgagcgcctccgtgggggacagagtgacaatcacatgtaaagcctcccaggacgtgggcactgccgtggcctggtaccagaaaaaaccgggaaaagcccctaagctgctgatctactgggccagcaccagacacaccggcgtccccgatagattcagcggctctggcagcggaactgatttcaccctgaccatttcttctctgcaggccgaggacttcgccgtgtacttttgccaccagcacagcagcaaccctctgaccttcggacagggcacaaagctggaaatcaagcggacagtggctgctccttctgtgttcatctttccacctagcgacgagcagctgaagagcggcaccgcctctgtggtgtgcctgctgaacaacttctaccccagagaagccaaagtgcagtggaaggtggacaacgccctgcaatctggcaacagccaggagagcgtgacggaacaagatagcaaggacagcacctactccctgagcagcacactgaccttgtccaaggcagattacgagaagcacaaggtgtacgcctgcgaggtgacccaccagggactgagcagcccagtgaccaagagcttcaacagaggagagtgcggcggcggcggaagcggaggcggaggcagcggcggcggcggcagtggaggcggcggctctggcggagggggcagtggcggtggcggatccggcggcggcggcagcgaggtgcagcttgtggaatccggcggcggcctggtgaagcccggcggtagcctgagactgtcttgtgccgcctctggcttcacctttagcaattacgccatgagctgggtgcgggaggctcccggcaaaagactggaatgggtcgccaccatcagcaaccggggatcatatacctactaccctgatagcgtgaaaggcaggttcacaatcagccgggacaatgccaagaacagcctgtacctgcagatgaactcactgcgggccgaggacaccgccctgtattactgcgccagagagagacctatggactactggggccagggcaccctggtgaccgtttcctccgccagcaccaagggccctagcgtgttccctctggccccatgcagcagaagcacatctgagagcaccgccgctctgggctgcctggtgaaggactacttccccgagcctgtgacagtgagctggaactccggcgccctgaccagcggcgtgcacacatttccagctgtgctgcagtctagcggcctgtacagcctgagcagcgttgtgacagtgccttctagcagcctcggcaccaagacctacacctgtaacgtggatcataagccttctaataccaaggttgacaagagagtgtga

SEQ ID NO: 27 - DNA, 3869 bp, synthetic construct
  source 1..3869: mol_type = other DNA
tcacactctcttgtcaaccttggtattagaaggcttatgatccacgttacaggtgtaggtcttggtgccgaggctgctagaaggcactgtcacaacgctgctcaggctgtacaggccgctagactgcagcacagctggaaatgtgtgcacgccgctggtcagggcgccggagttccagctcactgtcacaggctcggggaagtagtccttcaccaggcagcccagagcggcggtgctctcagatgtgcttctgctgcatggggccagagggaacacgctagggcccttggtgctggcggaggaaacggtcaccagggtgccctggccccagtagtccataggtctctctctggcgcagtaatacagggcggtgtcctcggcccgcagtgagttcatctgcaggtacaggctgttcttggcattgtcccggctgattgtgaacctgcctttcacgctatcagggtagtaggtatatgatccccggttgctgatggtggcgacccattccagtcttttgccgggagcctcccgcacccagctcatggcgtaattgctaaaggtgaagccagaggcggcacaagacagtctcaggctaccgccgggcttcaccaggccgccgccggattccacaagctgcacctcgctgccgccgccgccggatccgccaccgccactgccccctccgccagagccgccgcctccactgccgccgccgccgctgcctccgcctccgcttccgccgccgccgcactctcctctgttgaagctcttggtcactgggctgctcagtccctggtgggtcacctcgcaggcgtacaccttgtgcttctcgtaatctgccttggacaaggtcagtgtgctgctcagggagtaggtgctgtccttgctatcttgttccgtcacgctctcctggctgttgccagattgcagggcgttgtccaccttccactgcactttggcttctctggggtagaagttgttcagcaggcacaccacagaggcggtgccgctcttcagctgctcgtcgctaggtggaaagatgaacacagaaggagcagccactgtccgcttgatttccagctttgtgccctgtccgaaggtcagagggttgctgctgtgctggtggcaaaagtacacggcgaagtcctcggcctgcagagaagaaatggtcagggtgaaatcagttccgctgccagagccgctgaatctatcggggacgccggtgtgtctggtgctggcccagtagatcagcagcttaggggcttttcccggttttttctggtaccaggccacggcagtgcccacgtcctgggaggctttacatgtgattgtcactctgtcccccacggaggcgctcagggtgctagggctctgtgtcatctggatgtcgccggtggtgtcaggcagccacagcagcagcaggaacagcagctgagcgggggcttccatggtgggctagttggcgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgccagagcccaccatggaagcccctgcccagctgctgttcctgctgctcctgtggctgcctgacaccaccggcgatatcgtgctgacgcagagccctgatagcctggccgtgagcctcggcgaacgggccacaatcagctgtaaagcctctcagagcgtggactatgacggcgacagctacatgaactggtaccaggagaaacccggccagcctccaaaaatcctgatctacgacgccagcaatctggaaagcggcatccccgccagattcagcggcagcggctctggcactgatttcaccctgacaatttcttctctggaacccgaggattttgccatctactactgccagcagagcaacgaggacccctggacctttggaggcggcaccaaggtggaaatcaagcgcaccgtggccgccccttctgtctttatcttccctccaagcgacgagcagctgaagagcggaaccgcctctgtggtgtgcctgctgaataacttctaccccagagaggcaaaggtccaatggaaagttgacaacgccctgcaaagcggcaacagccaagagagcgtgaccgagcaggacagcaaggactcaacatacagcctgtccagcaccctgaccttgagcaaggccgactacgagaagcacaaggtgtacgcctgtgaagtgacacatcagggcctgtcctctcctgtgaccaaaagcttcaaccggggcgagtgcggcggcggcggctccggcggaggaggcagcggtggcggcggctctgggggaggcggaagcggcggaggcggcagcggcggaggcggcagcggcggcggcggatcccaggtgcagctggtgcagagcggagccgaggtgaaaaagcctggcgctagtgttaagctgagctgcaccgccagcggcttcaatatcaaggacgattacatccactgggtgaagaaggcccccggccagggactggagtggatcggcagaatcgaccctgccgacggccacacaaagtacgcccctaagttccaggtgaaagtcaccatcaccgccgataccagcacctctacagcttatctggaactgagcagccttagatccgaggacaccgctgtgtactactgcgccagatacggctacggcagagaagtgttcgactactggggacagggcacaacagtgaccgtgtcgtccgccagcacaaagggccctagcgtgttcccactggctccttgtagcagaagtacctcagagagcacggctgctctgggctgcctggtcaaggattatttccctgagcctgtgaccgtgtcctggaacagcggagccctgacaagcggggtgcacaccttccccgccgtgctgcagagcagcggcctgtactctctgtcctctgtcgtgacagtgcctagctctagcctgggcacaaagacctacacctgcaacgtggaccacaagcccagcaacaccaaggtggataagcgggtgtga

SEQ ID NO: 28 - DNA, 208 bp, synthetic construct
  source 1..208: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 29 - protein, 50 aa, synthetic construct
  VARIANT 6..50: note = "GGGGS" may or may not be present
GGGGSGGGGSGGGGSGGGGSGGGGSGGGGSGGGGSGGGGSGGGGSGGGGS

SEQ ID NO: 30 - DNA, 947 bp, synthetic construct
  source 1..947: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcc

SEQ ID NO: 31 - DNA, 4665 bp, synthetic construct
  source 1..4665: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccttacaattctagttccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacaggtttaaaccctgcagggagctctcacacccgcttatccaccttggtgttgctgggcttgtggtccacgttgcaggtgtaggtctttgtgcccaggctagagctaggcactgtcacgacagaggacagagagtacaggccgctgctctgcagcacggcggggaaggtgtgcaccccgcttgtcagggctccgctgttccaggacacggtcacaggctcagggaaataatccttgaccaggcagcccagagcagccgtgctctctgaggtacttctgctacaaggagccagtgggaacacgctagggccctttgtgctggcggacgacacggtcactgttgtgccctgtccccagtagtcgaacacttctctgccgtagccgtatctggcgcagtagtacacagcggtgtcctcggatctaaggctgctcagttccagataagctgtagaggtgctggtatcggcggtgatggtgactttcacctggaacttaggggcgtactttgtgtggccgtcggcagggtcgattctgccgatccactccagtccctggccgggggcctgcttcacccagtggatgtaatcgtccttgatattgaagccgctggcggtgcagctcagcttaacactagcgccaggctttttcacctcggctccgctctgcaccagctgcacctgggatccgccgccgccgctgccgcctccgccgctgccgcctccgccgcttccgcctcccccagagccgccgccaccgctgcctcctccgccggagccgccgccgccgcactcgccccggttgaagcttttggtcacaggagaggacaggccctgatgtgtcacttcacaggcgtacaccttgtgcttctcgtagtcggccttgctcaaggtcagggtgctggacaggctgtatgttgagtccttgctgtcctgctcggtcacgctctcttggctgttgccgctttgcagggcgttgtcaactttccattggacctttgcctctctggggtagaagttattcagcaggcacaccacagaggcggttccgctcttcagctgctcgtcgcttggagggaagataaagacagaaggggcggccacggtgcgcttgatttccaccttggtgccgcctccaaaggtccaggggtcctcgttgctctgctggcagtagtagatggcaaaatcctcgggttccagagaagaaattgtcagggtgaaatcagtgccagagccgctgccgctgaatctggcggggatgccgctttccagattgctggcgtcgtagatcaggatttttggaggctggccgggtttctgctggtaccagttcatgtagctgtcgccgtcatagtccacgctctgagaggctttacagctgattgtggcccgttcgccgaggctcacggccaggctatcagggctctgcgtcagcacgatatcgccggtggtgtcaggcagccacaggagcagcaggaacagcagctgggcaggggcttccatggtgggctctggcgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgccaactagcccaccatggaagcccctgcccagctgctgttcctgctgctcctgtggctgcctgacaccaccggcgatatcgtgctgacgcagagccctgatagcctggccgtgagcctcggcgaacgggccacaatcagctgtaaagcctctcagagcgtggactatgacggcgacagctacatgaactggtaccagcagaaacccggccagcctccaaaaatcctgatctacgacgccagcaatctggaaagcggcatccccgccagattcagcggcagcggctctggcactgatttcaccctgacaatttcttctctggaacccgaggattttgccatctactactgccagcagagcaacgaggacccctggacctttggaggcggcaccaaggtggaaatcaagcgcaccgtggccgccccttctgtctttatcttccctccaagcgacgagcagctgaagagcggaaccgcctctgtggtgtgcctgctgaataacttctaccccagagaggcaaaggtccaatggaaagttgacaacgccctgcaaagcggcaacagccaagagagcgtgaccgagcaggacagcaaggactcaacatacagcctgtccagcaccctgaccttgagcaaggccgactacgagaagcacaaggtgtacgcctgtgaagtgacacatcagggcctgtcctctcctgtgaccaaaagcttcaaccggggcgagtgcggcggcggcggctccggcggaggaggcagcggtggcggcggctctgggggaggcggaagcggcggaggcggcagcggcggaggcggcagcggcggcggcggatcccaggtgcagctggtgcagagcggagccgaggtgaaaaagcctggcgctagtgttaagctgagctgcaccgccagcggcttcaatatcaaggacgattacatccactgggtgaagcaggcccccggccagggactggagtggatcggcagaatcgaccctgccgacggccacacaaagtacgcccctaagttccaggtgaaagtcaccatcaccgccgataccagcacctctacagcttatctggaactgagcagccttagatccgaggacaccgctgtgtactactgcgccagatacggctacggcagagaagtgttcgactactggggacagggcacaacagtgaccgtgtcgtccgccagcacaaagggccctagcgtgttcccactggctccttgtagcagaagtacctcagagagcacggctgctctgggctgcctggtcaaggattatttccctgagcctgtgaccgtgtcctggaacagcggagccctgacaagcggggtgcacaccttccccgccgtgctgcagagcagcggcctgtactctctgtcctctgtcgtgacagtgcctagctctagcctgggcacaaagacctacacctgcaacgtggaccacaagcccagcaacaccaaggtggataagcgggtgtgagagctccctgcagggtttaaacctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaactagaaattaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 32 - DNA, 4618 bp, synthetic construct
  source 1..4618: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccttacaattctagttccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacaggtttaaaccctgcagggagctcgtcacactctcttgtcaaccttggtattagaaggcttatgatccacgttacaggtgtaggtcttggtgccgaggctgctagaaggcactgtcacaacgctgctcaggctgtacaggccgctagactgcagcacagctggaaatgtgtgcacgccgctggtcagggcgccggagttccagctcactgtcacaggctcggggaagtagtccttcaccaggcagcccagagcggcggtgctctcagatgtgcttctgctgcatggggccagagggaacacgctagggcccttggtgctggcggaggaaacggtcaccagggtgccctggccccagtagtccataggtctctctctggcgcagtaatacagggcggtgtcctcggcccgcagtgagttcatctgcaggtacaggctgttcttggcattgtcccggctgattgtgaacctgcctttcacgctatcagggtagtaggtatatgatccccggttgctgatggtggcgacccattccagtcttttgccgggagcctgccgcacccagctcatggcgtaattgctaaaggtgaagccagaggcggcacaagacagtctcaggctaccgccgggcttcaccaggccgccgccggattccacaagctgcacctcgctgccgccgccgccggatccgccaccgccactgccccctccgccagagccgccgcctccactgccgccgccgccgctgcctccgcctccgcttccgccgccgccgcactctcctctgttgaagctcttggtcactgggctgctcagtccctggtgggtcacctcgcaggcgtacaccttgtgcttctcgtaatctgccttggacaaggtcagtgtgctgctcagggagtaggtgctgtccttgctatcttgttccgtcacgctctcctggctgttgccagattgcagggcgttgtccaccttccactgcactttggcttctctggggtagaagttgttcagcaggcacaccacagaggcggtgccgctcttcagctgctcgtcgctaggtggaaagatgaacacagaaggagcagccactgtccgcttgatttccagctttgtgccctgtccgaaggtcagagggttgctgctgtgctggtggcaaaagtacacggcgaagtcctcggcctgcagagaagaaatggtcagggtgaaatcagttccgctgccagagccgctgaatctatcggggacgccggtgtgtctggtgctggcccagtagatcagcagcttaggggcttttcccggtttttgctggtaccaggccacggcagtgcccacgtcctgggaggctttacatgtgattgtcactctgtcccccacggaggcgctcagggtgctagggctctgtgtcatctggatgtcgccggtggtgtcaggcagccacagcagcagcaggaacagcagctgagcgggggcttccatggtgggctctggcgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgccaactagcccaccatggaagcccccgctcagctgctgttcctgctgctgctgtggctgcctgacaccaccggcgacatccagatgacacagagccctagcaccctgagcgcctccgtgggggacagagtgacaatcacatgtaaagcctcccaggacgtgggcactgccgtggcctggtaccagcaaaaaccgggaaaagcccctaagctgctgatctactgggccagcaccagacacaccggcgtccccgatagattcagcggctctggcagcggaactgatttcaccctgaccatttcttctctgcaggccgaggacttcgccgtgtacttttgccaccagcacagcagcaaccctctgaccttcggacagggcacaaagctggaaatcaagcggacagtggctgctccttctgtgttcatctttccacctagcgacgagcagctgaagagcggcaccgcctctgtggtgtgcctgctgaacaacttctaccccagagaagccaaagtgcagtggaaggtggacaacgccctgcaatctggcaacagccaggagagcgtgacggaacaagatagcaaggacagcacctactccctgagcagcacactgaccttgtccaaggcagattacgagaagcacaaggtgtacgcctgcgaggtgacccaccagggactgagcagcccagtgaccaagagcttcaacagaggagagtgcggcggcggcggaagcggaggcggaggcagcggcggcggcggcagtggaggcggcggctctggcggagggggcagtggcggtggcggatccggcggcggcggcagcgaggtgcagcttgtggaatccggcggcggcctggtgaagcccggcggtagcctgagactgtcttgtgccgcctctggcttcacctttagcaattacgccatgagctgggtgcggcaggctcccggcaaaagactggaatgggtcgccaccatcagcaaccggggatcatatacctactaccctgatagcgtgaaaggcaggttcacaatcagccgggacaatgccaagaacagcctgtacctgcagatgaactcactgcgggccgaggacaccgccctgtattactgcgccagagagagacctatggactactggggccagggcaccctggtgaccgtttcctccgccagcaccaagggccctagcgtgttccctctggccccatgcagcagaagcacatctgagagcaccgccgctctgggctgcctggtgaaggactacttccccgagcctgtgacagtgagctggaactccggcgccctgaccagcggcgtgcacacatttccagctgtgctgcagtctagcggcctgtacagcctgagcagcgttgtgacagtgccttctagcagcctcggcaccaagacctacacctgtaacgtggatcataagccttctaataccaaggttgacaagagagtgtgagagctccctgcagggtttaaacctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaactagaaattaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 33 - DNA, 4811 bp, synthetic construct
  source 1..4811: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccttacaattctagttccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacaggtttaaaccctgcagggagctctcacacccgcttatccaccttggtgttgctgggcttgtggtccacgttgcaggtgtaggtctttgtgcccaggctagagctaggcactgtcacgacagaggacagagagtacaggccgctgctctgcagcacggcggggaaggtgtgcaccccgcttgtcagggctccgctgttccaggacacggtcacaggctcagggaaataatccttgaccaggcagcccagagcagccgtgctctctgaggtacttctgctacaaggagccagtgggaacacgctagggccctttgtgctggcggacgacacggtcactgttgtgccctgtccccagtagtcgaacacttctctgccgtagccgtatctggcgcagtagtacacagcggtgtcctcggatctaaggctgctcagttccagataagctgtagaggtgctggtatcggcggtgatggtgactttcacctggaacttaggggcgtactttgtgtggccgtcggcagggtcgattctgccgatccactccagtccctggccgggggcctgcttcacccagtggatgtaatcgtccttgatattgaagccgctggcggtgcagctcagcttaacactagcgccaggctttttcacctcggctccgctctgcaccagctgcacctgggatccgccgccgccgctgccgcctccgccgctgccgcctccgccgcttccgcctcccccagagccgccgccaccgctgcctcctccgccggagccgccgccgccgcactcgccccggttgaagcttttggtcacaggagaggacaggccctgatgtgtcacttcacaggcgtacaccttgtgcttctcgtagtcggccttgctcaaggtcagggtgctggacaggctgtatgttgagtccttgctgtcctgctcggtcacgctctcttggctgttgccgctttgcagggcgttgtcaactttccattggacctttgcctctctggggtagaagttattcagcaggcacaccacagaggcggttccgctcttcagctgctcgtcgcttggagggaagataaagacagaaggggcggccacggtgcgcttgatttccaccttggtgccgcctccaaaggtccaggggtcctcgttgctctgctggcagtagtagatggcaaaatcctcgggttccagagaagaaattgtcagggtgaaatcagtgccagagccgctgccgctgaatctggcggggatgccgctttccagattgctggcgtcgtagatcaggatttttggaggctggccgggtttctgctggtaccagttcatgtagctgtcgccgtcatagtccacgctctgagaggctttacagctgattgtggcccgttcgccgaggctcacggccaggctatcagggctctgcgtcagcacgatatcgccggtggtgtcaggcagccacaggagcagcaggaacagcagctgggcaggggcttccatggtgggctctggcgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagccaactagcccaccatggaagcccccgctcagctgctgttcctgctgctgctgtggctgcctgacaccaccggcgacatccagatgacacagagccctagcaccctgagcgcctccgtgggggacagagtgacaatcacatgtaaagcctcccaggacgtgggcactgccgtggcctggtaccagcaaaaaccgggaaaagcccctaagctgctgatctactgggccagcaccagacacaccggcgtccccgatagattcagcggctctggcagcggaactgatttcaccctgaccatttcttctctgcaggccgaggacttcgccgtgtacttttgccaccagcacagcagcaaccctctgaccttcggacagggcacaaagctggaaatcaagcggacagtggctgctccttctgtgttcatctttccacctagcgacgagcagctgaagagcggcaccgcctctgtggtgtgcctgctgaacaacttctaccccagagaagccaaagtgcagtggaaggtggacaacgccctgcaatctggcaacagccaggagagcgtgacggaacaagatagcaaggacagcacctactccctgagcagcacactgaccttgtccaaggcagattacgagaagcacaaggtgtacgcctgcgaggtgacccaccagggactgagcagcccagtgaccaagagcttcaacagaggagagtgcggcggcggcggaagcggaggcggaggcagcggcggcggcggcagtggaggcggcggctctggcggagggggcagtggcggtggcggatccggcggcggcggcagcgaggtgcagcttgtggaatccggcggcggcctggtgaagcccggcggtagcctgagactgtcttgtgccgcctctggcttcacctttagcaattacgccatgagctgggtgcggcaggctcccggcaaaagactggaatgggtcgccaccatcagcaaccggggatcatatacctactaccctgatagcgtgaaaggcaggttcacaatcagccgggacaatgccaagaacagcctgtacctgcagatgaactcactgcgggccgaggacaccgccctgtattactgcgccagagagagacctatggactactggggccagggcaccctggtgaccgtttcctccgccagcaccaagggccctagcgtgttccctctggccccatgcagcagaagcacatctgagagcaccgccgctctgggctgcctggtgaaggactacttccccgagcctgtgacagtgagctggaactccggcgccctgaccagcggcgtgcacacatttccagctgtgctgcagtctagcggcctgtacagcctgagcagcgttgtgacagtgccttctagcagcctcggcaccaagacctacacctgtaacgtggatcataagccttctaataccaaggttgacaagagagtgtgagagctccctgcagggtttaaacctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaactagaaattaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa